SEQUENCE LISTING (28 sequences)

SEQ ID NO: 1 - DNA, 775 bp, synthetic construct
  source 1..775: mol_type = other DNA
ttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttacgggactttcctacttggcagtacatctccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggggagtcgctgcgttgccttcgccccgtgccccgctccgcgccgcctcgcgccgcccgccccggctctgactgaccgcgttactcccacaggtgagcgggcgggacggcccttctcctccgggctgtaattagcaagaggtaagggtttaagggatggttggttggtggggtattaatgttta

SEQ ID NO: 2 - DNA, 813 bp, synthetic construct
  source 1..813: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcagc

SEQ ID NO: 3 - DNA, 837 bp, synthetic construct
  source 1..837: mol_type = other DNA
ttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttacgggactttcctacttggcagtacatctgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcagc

SEQ ID NO: 4 - DNA, 795 bp, synthetic construct
  source 1..795: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcagc

SEQ ID NO: 5 - DNA, 4639 bp, synthetic construct
  source 1..4639: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgatatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 6 - DNA, 5992 bp, synthetic construct
  source 1..5992: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgatatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactagtcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatgatcgaaacatacaaccaaacttctccccgatctgcggccactggactgcccatcagcatgaaaatttttatgtatctccttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagatcaaaagccagtttgaaggctttgtgaaggatataatgttaaacaaagaggagacgaagaaagaaaacagcggagatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccctttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgatctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 7 - DNA, 4639 bp, synthetic construct
  source 1..4639: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgatatcctgagcagactggacccacctgaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 8 - DNA, 5992 bp, synthetic construct
  source 1..5992: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgatatcctgagcagactggacccacctgaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactagtcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatgatcgaaacatacaaccaaacttctccccgatctgcggccactggactgcccatcagcatgaaaatttttatgtatctccttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagatcaaaagccagtttgaaggctttgtgaaggatataatgttaaacaaagaggagacgaagaaagaaaacagcggagatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccctttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgatctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 9 - DNA, 2950 bp, synthetic construct
  source 1..2950: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggattttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatttacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatttaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagattctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagcggcagcggctacatccccgaggcccccagagacggccaggcctacgtgagaaaggacggcgagtgggtgctgctgagcaccttcctgggctaaactagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 10 - DNA, 4303 bp, synthetic construct
  source 1..4303: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggattttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatttacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatttaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagattctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagcggcagcggctacatccccgaggcccccagagacggccaggcctacgtgagaaaggacggcgagtgggtgctgctgagcaccttcctgggctaaactagtcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatgatcgaaacatacaaccaaacttctccccgatctgcggccactggactgcccatcagcatgaaaatttttatgtatctccttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagatcaaaagccagtttgaaggctttgtgaaggatataatgttaaacaaagaggagacgaagaaagaaaacagcggagatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccctttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgatctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 11 - protein, 5 aa, synthetic construct
CRGDC

SEQ ID NO: 12 - protein, 9 aa, synthetic construct
CXCRGDCXC

SEQ ID NO: 13 - protein, 9 aa, synthetic construct
CDCRGDCFC

SEQ ID NO: 14 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - DNA, 3822 bp, synthetic construct
  source 1..3822: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgatatcctgagcagactggacccacctgaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 16 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - DNA, 3813 bp, synthetic construct
  source 1..3813: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatcagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccacctacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgacgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccacagaagagccagaagcgtggccagccagagcatcatcgcctacaccatgagcctgggcgccgagaacagcgtggcctacagcaacaacagcatcgccatccccatcaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgatatcctggccagactggacccacctgaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccacccacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 18 - protein, 1270 aa, synthetic construct
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - DNA, 3813 bp, synthetic construct
  source 1..3813: mol_type = other DNA
atgttcgtgttcctggtgctgctgcccctggtgagcagccagtgcgtgaactttaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgccaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagaggcctgccccagggcttcagcgccctggagcccctggtggacctgcccatcggcatcaacatcaccagattccagaccctgcacattagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaacatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtgaagggcttcaactgctacttccccctgcagagctacggcttccagcccacctacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgtggagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgatatcctgagcagactggacccacctgaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 20 - protein, 1270 aa, synthetic construct
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - DNA, 3822 bp, synthetic construct
  source 1..3822: mol_type = other DNA
atgttcgtgttcctggtgctgctgcccctggtgagcagccagtgcgtgaacttcaccaacagaacccagctgcccagcgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaactaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagcgagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaccatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtgaagggcttcaactgctacttccccctgcagagctacggcttccagcccacctacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagtacgtgaacaacagctacgagtgcgacatccccatcggcgccggcatctgcgccagctaccagacccagaccaacagccccagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgatatcctgagcagactggacccacctgaggccgaggtgcagatcgacagactgatcaccggcagactgcagagcctgcagacctacgtgacccagcagctgatcagagccgccgagatcagagccagcgccaacctggccgccatcaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcttcgtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 22 - protein, 1273 aa, synthetic construct
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - DNA, 3814 bp, synthetic construct
  source 1..3814: mol_type = other DNA
atgttcgtgttcctggtgctgctgcccctggtgagcagccagtgcgtgaacctgcgcaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcggagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaacatcgccgactacaactacaagctgcccgacgacttcaccggctgcgtgatcgcctggaacagcaacaacctggacagcaaggtgggcggcaactacaactaccggtacagactgttcagaaagagcaacctgaagcccttcgagagagacatcagcaccgagatctaccaggccggcagcaagccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccggagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcagaacgtggtgaaccagaacgcccaggccctgaacaccctggtgaagcagctgagcagcaacttcggcgccatcagcagcgtgctgaacgatatcctgagcagactggacccacctgaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacta

SEQ ID NO: 24 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - DNA, 3816 bp, synthetic construct
  source 1..3816: mol_type = other DNA
atgttcgtgttcctggtgctgctgcccctggtgagcagccagtgcgtgaacctgcgcaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcggagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaacatcgccgactacaactacaagctgcccgacgacttcaccggctgcgtgatcgcctggaacagcaacaacctggacagcaaggtgggcggcaactacaactaccggtacagactgttcagaaagagcaacctgaagcccttcgagagagacatcagcaccgagatctaccaggccggcagcaagccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccggggaagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcagaacgtggtgaaccagaacgcccaggccctgaacaccctggtgaagcagctgagcagcaacttcggcgccatcagcagcgtgctgaacgatatcctgagcagactggacccacctgaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 26 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - DNA, 3813 bp, synthetic construct
  source 1..3813: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgtgatcagcggcaccaacggcaccaagagattcgacaaccccgtgctgcccttcaacgacggcgtgtacttcgccagcatcgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcatcgtggagcctgagagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcgacgaggtgttcaacgccaccagattcgccagcgtgtacgcctggaacagaaagagaatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatcagaggcgacgaggtgagacagatcgcccccggccagaccggcaacatcgccgactacaactacaagctgcccgacgacttcaccggctgcgtgatcgcctggaacagcaacaagctggacagcaaggtgagcggcaactacaactacctgtacagactgttcagaaagagcaacctgaagcccttcgagagagacatcagcaccgagatctaccaggccggcaacaagccctgcaacggcgtggccggcttcaactgctacttccccctgaagagctacagcttccggcccacctacggcgtgggccaccagccctacagagtggtggtgctgagcttcgagctgctgcacgcccccgccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagtacgtgaacaacagctacgagtgcgacatccccatcggcgccggcatctgcgccagctaccagacccagaccaagagccacggaagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagagccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaagggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgatatcttcagcagactggacccacctgaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaatttaaatgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 28 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVEPERDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2025/0345414 A1
(43) Pub. Date: Nov. 13, 2025

(54) ADENOVIRAL VECTOR-BASED VACCINE FOR EMERGING VIRUSES

(71) Applicants: THERAVAX, INC., Wilmington, DE (US); FUNDACION INSTITUTO LELOIR, Ciudad Autonoma de Buenos Aires (AR); CONSEJO NACIONAL DE INVESTIGACIONES CIENTIFICAS Y TECNICAS (CONICET), Ciudad Autonoma de Buenos Aires (AR)

(72) Inventors: Norberto Julián Maggini, City of Buenos Aires (AR); Osvaldo Luis Podhajcer, City of Buenos Aires (AR); Maria Veronica Lopez, City of Buenos Aires (AR); Sabrina Eugenia Vinzon, City of Buenos Aires (AR); Eduardo Gustavo Alfredo Cafferata, City of Buenos Aires (AR); Felipe Javier Núñez Aguilera, Ciudad Autonoma de Buenos Aires (AR); Paula Berguer, Buenos Aires (AR); Maximiliano Sanchez Lamas, Buenos Aires (AR)

(21) Appl. No.: 18/681,226

(22) PCT Filed: Aug. 5, 2022
(86) PCT No.: PCT/US2022/074573

§ 371 Date: Feb. 5, 2024

Related U.S. Application Data
(60) Provisional application No. 63/230,284, filed on Aug. 6, 2021.

(51) Int. Cl. A61K 39/215; A61K 39/00; A61P 31/14; C07K 14/005; C07K 14/705; C12N 15/86

(52) U.S. Cl. CPC A61K 39/215; A61P 31/14; C07K 14/005; C07K 14/70575; C12N 15/86; A61K 2039/5254; A61K 2039/5256; A61K 2039/545; A61K 2039/575; C12N 2710/10022; C12N 2710/10034; C12N 2710/10043; C12N 2770/20022; C12N 2770/20034; C12N 2830/48; C12N 2840/203

(57) ABSTRACT

Provided herein is an adenoviral vector-based vaccine for inducing immune responses against viruses, such as coronaviruses. The adenoviral vector comprises a hybrid promoter, a nucleic acid sequence encoding a viral antigen operatively linked to the hybrid promoter; a posttranscriptional regulatory element; and a modified fiber protein. Also provided is a method of inducing an immune response against a coronavirus using a composition containing the adenoviral vector.

### STATEMENT REGARDING RELATED APPLICATIONS

This application is a national phase application under 35 U.S.C. § 371 of PCT International Application No. PCT/US2022/074573, filed on Aug. 5, 2022, which claims the benefit of U.S. Provisional Patent Application No. 63/230,284, filed Aug. 6, 2021, the entire contents of which are incorporated herein by reference for all purposes.

### SEQUENCE LISTING

The computer readable sequence listing filed herewith, titled “THVAX_39591_252_SequenceListing”, created Mar. 13, 2025, having a file size of 93,830 bytes, is hereby incorporated by reference in its entirety.

### BACKGROUND

The disease caused by the novel severe acute respiratory syndrome coronavirus 2 (SARS-CoV-2) that appeared in Wuhan, China in December 2019, and declared a pandemic by the World Health Organization (WHO) in March 2020, has had an effect of enormous proportions, globally leading to more than 170 million confirmed cases and causing more than 3.5 million deaths (covid19.who.int/). The U.S. Food and Drug Administration (FDA) and the European Medicinal Agency (EMA), among other regulatory bodies, have issued emergency use authorization for different vaccines to prevent COVID-19, which are based mainly on mRNA and adenoviral-based platforms (Bouazzaoui, Abdellatif et al. 2021, Kyriakidis, López-Cortés et al. 2021, van de Berg, Kis et al. 2021). The approved vaccines developed by Pfizer and Moderna are based on mRNA technology encapsulated in lipid nanoparticles, while the approved vaccines of AstraZeneca, Johnson and Johnson, and the Gamaleya Institute are based on replication-incompetent adenoviral vectors (Tregoning, Brown et al. 2020, Zhao, Zhao et al. 2020, Rawat, Kumari et al. 2021).

With the exception of the hAdV26-based vaccine of Johnson & Johnson, all of the vaccines are given as a prime-boost approach to achieve maximal immune response and protection (Chakraborty, Mallajosyula et al. 2021). The Johnson & Johnson vaccine was approved for one dose with an immunization capacity slightly over 60% (Sadoff, Le Gars et al. 2021). Companies are facing challenges in manufacturing COVID-19 vaccines and building the supply chains to meet the demand; in fact, the U.K. and Canada health authorities prioritized distribution of a first vaccine dose to as many people as possible (gov.uk/government/publications/uk-covid-19-vaccines-delivery-plan/uk-covid-19-vaccines-delivery-plan; canada.ca/en/public-health/services/immunization/national-advisory-committee-on-immunization-naci/recommendations-use-covid-19-vaccines.html). The need for two doses and the fact that mRNA vaccines require using logistically challenging cold-chains make mRNA vaccines more challenging to deploy in developing countries where ultra-low freezers may not be widely available.

Moreover, the emergence of variants of concern (VOCs) has challenged the efficacy of existing vaccines for coronavirus. The main characteristic of emerging VOCs, especially Delta and currently Omicron, is their incremental spreading and dramatic immune escape from sera of vaccinated individuals and monoclonal antibodies that explain the increased rate of breakthrough infections^{8, 9}. Phylogenetic analyses of Delta and Omicron indicate that these lineages might not directly derive from previous VOCs (Alpha, Beta or Gamma)⁸. The mutational landscape of Omicron BA.1 (29 amino acid substitutions, 3 deletions and 3 insertions only in Spike) and its sub-variants¹⁰ also suggest a long and complex evolutionary process. Of note, 15 of the Omicron mutations are in the RBD motif that interacts with ACE-2¹¹. Despite that divergent mutational landscape, a closer analysis demonstrates that several VOCs share specific mutations in the RBD region. In addition to the fact that all VOCs share D614G, Beta and Gamma share the RBD mutations K417N, E484K and N501Y; the N501Y mutation is also shared by Alpha and Omicron¹². Omicron also shows mutations in K417 and E484, although different from those observed in the other VOCs¹³. N501Y or the combination of N501Y, K417T and E484K showed stronger affinity to ACE-2 compared with the B.1 sequence¹⁴. Alpha and Omicron BA.1 share the deletion in amino acids 69 and 70¹³. The high mutation rate of the virus suggests the likely possibility of the emergence of novel variants in the near future stressing the need for vaccines with broader coverage.

Accordingly, there remains a need for compositions and methods for vaccination against COVID-19, in particular vaccinations that remain effective against multiple VOCS, and other emerging viruses, that may be widely deployed in developing nations. Specifically, there is a need for effective vaccines that prevent not only infection but also transmission of the virus.

### BRIEF SUMMARY OF THE DISCLOSURE

The disclosure provides a chimeric, replication incompetent adenoviral vector comprising: (a) a hybrid promoter comprising an exogenous intron; (b) a nucleic acid sequence encoding a viral antigen operatively linked to the hybrid promoter; (c) a post-transcriptional regulatory element; and (d) a modified fiber protein.

The disclosure also provides a method of inducing an immune response against a coronavirus in a subject, which comprises administering to the subject a composition comprising a pharmaceutically acceptable carrier and an adenoviral vector described herein. In some embodiments, following administration of the composition the nucleic acid sequence encoding a coronavirus spike protein is expressed in the subject, thereby inducing an immune response against the coronavirus in the subject.

Also provided is the use of a composition in the preparation of a medicament for immunizing a subject against a coronavirus, wherein the composition comprises a pharmaceutically acceptable carrier and an adenoviral vector as described herein. In some embodiments, the composition is provided to the subject intranasally to induce an immune response in the subject.

### BRIEF DESCRIPTION OF THE DRAWING(S)

FIGS. 1A-1C are graphs showing luciferase expression following cell transfection or transduction with adenoviral vectors. In FIG. 1A, 293T cells were transiently transfected with plasmid vectors comprising different promoters upstream of luciferase. An empty vector was included as a control. The promoter activity was evaluated by luminescence, using a luciferase gene reporter assay. The relative luciferase units were normalized by firefly renilla and expressed as fold induction over the SV-40 promoter. Bars represent mean±SEM (n=3) (****P<0.0001; one-way ANOVA). In FIG. 1B and FIG. 1C, HS729T (1B) and THP-1 (1C) were transduced with replication-deficient adenovirus, hAdV5-Renilla, and hAdV5/3-Renilla. Luciferase activity was determined 48 hours post-transduction. Results are expressed in relative luciferase units (RLU) of firefly renilla normalized to hAd5-Renilla. Bars represent mean±SEM (n=3) (****P<0.0001; t-student).

FIGS. 2A-2C show spike expression following in vitro transduction with AdV-vectored vaccines. FIGS. 2A-2C are graphs showing results of western blot assays performed on HS729T (FIG. 2A), THP1 (FIG. 2B), and immature dendritic (iDC) (FIG. 2C) cells after 48 hours of transduction with CoroVaxG.5, CoroVaxG.3, and control Ad.C. Spike expression was detected using an anti-spike RBD antibody. β-actin was used as a loading control. FIG. 2D, FIG. 2E, and FIG. 2F are bar graphs showing results of semi-quantification of western blot assays (n=3; ***P<0.001; one-way ANOVA).

FIGS. 3A-3D illustrate the immunogenicity and long-term humoral response induced by Ad-vectored vaccines. FIG. 3A is a diagram of the schedule of immunization and sample collection. Six-week-old Balb/c mice (n=5/group) received immunizations with 10⁹ or 10¹⁰ vp of the adenoviral vectors CoroVaxG.3 and CoroVaxG.5. Serum was collected and assessed at the indicated time points. Animals were euthanized at 140 days post vaccination (long term). FIG. 3B is a graph showing anti-S IgG titers induced by the tested vaccine constructs (data are mean±SEM (standard error of the mean). FIG. 3C and FIG. 3D are graphs showing anti-S IgG1 and IgG2a concentrations 28 days post-vaccination. Blue symbols: CoroVaxG.5; red symbols: CoroVaxG.3, grey symbols: Ad.C; light symbols: 10⁹ vp; dark symbols: 10¹⁰ vp (box plots show median, 25^{th}, and 75th percentiles, and the whiskers show the range).

FIGS. 4A-4D illustrate the cellular immune response induced by Ad-vectored vaccines. BALB/c mice received 10⁹ or 10¹⁰ vp of an Ad-vectored vaccine (blue: CoroVaxG.5; red: CoroVaxG.3; grey: Ad.C; green: Naïve) and sacrificed after 14 days (10¹⁰ vp) or 20 weeks (10⁹ vp). FIG. 4A and FIG. 4B are graphs illustrating cells secreting IFN-7 per million of splenocytes as determined by ELISPOT at 14 days (FIG. 4A) and 20 weeks (FIG. 4B) post immunization. Samples were analyzed in duplicate. Results of each group are expressed as mean of spot-forming units (SFU). For FACS analysis, splenocytes were stained with anti-CD8α, anti-CD62L and anti-CD44 fluorochrome-conjugated antibodies. Stained splenocytes were subjected to flow cytometry analysis to quantify memory T cells (TCM: CD44^{high} CD62L^{high} and TEM: CD44^{high} CD62L^{Low}), as shown in FIG. 4C and FIG. 4D. In FIG. 4C, data are expressed as % among total CD8+ cells; unstimulated controls (black dots) were included. FIG. 4D shows representative dot plots of each group. The gate shows TCM subpopulation. The box and whisker plots represent the median (mid-line), max and min (boxes) and range (whiskers). Vaccinated groups were always significantly different to the naive and unstimulated group; *p<0.05, **p<0.01; Kruskal-Wallis test with Dunn's multiple comparisons a posteriori.

FIGS. 5A-5C are graphs illustrating the elicitation of neutralizing antibodies by Ad-vectored vaccines. Sera from animals vaccinated with the higher dose were used to measure SARS-CoV-2 neutralizing antibodies by a Pseudovirus-Based Neutralization Assay, as shown in FIG. 5A. FIG. 5B shows the neutralization capacity of CoroVaxG.3 against variants of concern using a pseudovirus platform; while FIG. 5C shows the neutralization capacity against the authentic variant of concern gamma. Comparisons were performed by a Kruskal-Wallis test, followed by Dunn's multiple comparisons.

FIG. 6 is a schematic representation of all the constructed vaccines. S1 and S2 correspond to the Spike subunits. The dots in different colors are related to each one of the Spike domains: SS: Signal Sequence, NTD N-Terminal Domain, RBD: Receptor Binding Domain, RBM: Receptor Binding Motif, SD1: Subdomain 1, SD2: Subdomain 2, FP: Fusion Peptide, HR1: Heptad Repeat 1, CH: Central Helix, CD: Connector Domain, HR2: Heptad Repeat 2, TD: Transmembrane Domain, CT: Cytoplasmic Tail. Solid arrow: furin cleavage motif; Outlined arrow: S2 protease cleavage site.

FIG. 7 is a western blot showing Spike expression following in vitro transduction with each vaccine. Hs 297T cells were transduced with each vaccine at MOI 100; 48 h later Spike expression was detected using an anti-Spike specific antibody. β-actin was used as a loading control. The arrow shows the absence of the band in the Spike constructs expressing a mutated furin cleavage domain.

FIG. 8 shows immunogenicity of a single dose of CoroVax vaccine candidates. Groups of BALB/c mice (n=5) were immunized with a single intramuscular injection of CorovaxG.3 vaccine candidates based on different SARS-CoV-2 VOCs or with an empty adenovirus vector (Ad.C) or PBS (naïve). Sera at 4 weeks post immunization were collected. SARS-CoV-2 Spike-specific IgG titers were determined. Differences between experimental groups of animals were analyzed by Kruskal-Wallis test with Dunn's multiple comparisons a posteriori; * p<0.05, ** p<0.01.

FIG. 9 shows results from a pseudovirus-based Neutralization Assay against the homologous SARS-CoV-2 variant. Sera obtained from vaccinated mice was used to measure their neutralizing capacity. The box plots show the median, 25th and 75th percentiles, and the whiskers show the range. Comparisons were performed by a Kruskal-Wallis test, followed by Dunn's multiple comparisons.

FIG. 10 shows cellular immune response induced by the different vaccines. BALB/c mice received a 10⁹ dose of each vaccine and 28 days later mice were sacrificed and splenocytes were removed to assess for IFN-γ secreting cells using ELISPOT. The samples were analyzed in duplicates. SFU: mean of Spot Forming Units. AdC: control adenovirus.

FIG. 11A-11D show cross-neutralization of VOC-matched pseudoviruses by CoroVax vaccine variants. Sera from BALB/c mice immunized with a single injection of CorovaxG.3 vaccine candidates: (FIG. 11A) Corovax.G3, (FIG. 11B) Corovax.G3-P, (FIG. 11C) Corovax.G3-D, and (FIG. 11D) Corovax.G3-D.FR, were tested against pseudoviruses bearing B.1 (D614G), B.1.1.7 (Alpha), B.1.351 (Beta), P.1 (Gamma) and B.1.617.2 (Delta) spikes, and the calculated ID50s are depicted in each graph. The dashed line on each graph indicates the limit of detection. The differences in neutralization of different variant viruses are indicated by horizontal lines, and the fold differences in neutralization GMTs are shown.

FIG. 12 is a schematic representation of the protocol of mice challenge with each of the SARS-CoV-2 variants. Transgenic mice were vaccinated at day −28, followed by a challenge with the SARS-CoV-2 variant at day 0. Mice were sacrificed at day 4 to collect samples for viral load and the experiment was terminated for remaining mice at day 10 when mice health was irreversible.

FIG. 13A-13B show viral load following vaccination and challenge with the Gamma VOC. FIG. 13A shows viral load in lungs and FIG. 13B shows viral load in the brain. Transgenic mice vaccinated at day −28 were challenged with Gamma at day 0 and sacrificed at day +4. All the organs were removed and samples of lungs and brain were used to assess viral load by quantitative PCR of E gene levels. The box plots show the median, 25th and 75th percentiles, and the whiskers show the range. * p<0.05, **p<0.01 and ***p<0.001. FIG. 13C shows weight change as a percentage of initial weight.

FIG. 14A-14C show viral following vaccination and challenge with the Delta VOC. FIG. 14A shows viral load in lung. FIG. 14B shows subgenomic RNA levels, indicating a complete inhibition of viral replication in lungs. FIG. 14C shows viral load in brain. Transgenic mice vaccinated at day −28 were challenged with Delta at day 0 and sacrificed at day +4. All the organs were removed and samples of lungs and brain were used to assess viral load by quantitative PCR of E gene levels. The box plots show the median, 25th and 75th percentiles, and the whiskers show the range.* p<0.05, **p<0.01, ***p<0.001, **** p<0.0001. FIG. 14D shows weight loss as a percentage of initial weight.

FIG. 15 shows K18-hACE2 mice were vaccinated with Corovax.G3 candidates and serum samples collected 28 days post inoculation, right before challenge with a SARS-CoV-2 Delta isolate. Sera were used to measure SARS-CoV2 neutralizing antibodies by a Pseudovirus-Based Neutralization Assay against the Delta variant. The box plots show the median, 25th and 75th percentiles, and the whiskers show the range. Comparison was performed by a Mann-Whitney test (p<0.01).

FIG. 16 shows Omicron-specific neutralization of CoroVax.G3 vaccine variants. Sera from BALB/c mice immunized with a single injection of each vaccine candidates were tested against pseudoviruses bearing B.1.1.529.1 (Omicron BA.1) spike, and the calculated ID50s are depicted. The dashed line indicates the limit of detection.

FIG. 17 shows immunogenicity of an Omicron-based vaccine in a single dose regimen. BALB/c mice were immunized with a single dose of CorovaxG.3-O.FR vaccine candidate or with Ad.C vector. Endpoint IgG titers were determined by ELISA against Wu-1 Spike (left from the dashed line) or BA.1 Spike (right from the dashed line).

FIG. 18 shows immunogenicity of VOC-matched vaccines administered in a prime/boost regimen. BALB/c mice were immunized with a single dose of CorovaxG.3 followed by a boost 28 d later of either CoroVaxG.3, CoroVaxG.3-D.FR or CoroVaxG.3-O.FR. Endpoint IgG titers were determined by ELISA against Wu-1 Spike.

FIG. 19. Cross-neutralization of VOC-matched pseudoviruses by sera of animals boosted with Corovax.G3-D.FR. Sera from BALB/c mice immunized with a dose of CorovaxG.3 followed by a boost 28 d later of either Corovax.G3 or Corovax.G3-D.FR vaccines, were tested against pseudoviruses bearing B.1 (D614G), B.1.1.7 (Alpha), B.1.351 (Beta), P.1 (Gamma), B.1.617.2 (Delta) and B.1.1.529.1 (Omicron BA.1) spikes, and the calculated ID50s are depicted in the graph. The dashed line indicates the limit of detection.

FIG. 20 shows weight change following vaccination, boost, and challenge. BALB/c mice immunized with a dose of CorovaxG.3 followed by a boost 28 d later of either Corovax.G3, Corovax.G3-D.FR, or Corovax.G3-O.FR vaccines. Data are expressed as weight change in percentage of initial weight.

FIG. 21A-21D show, histopathological analysis and immunohistochemical assessment of SARS-CoV-2 transmission in the brain. FIG. 21A shows histopathological analysis of mice brain in unvaccinated mice challenged as described with the Gamma strain. At day 4 mice were euthanized and brain samples were stained with hematoxylin and eosin. The circles point to necrotic neurons with viral inclusions. FIG. 21B shows expression of the SARS-CoV2 Nucleocapsid (N) protein in mice brain (Day 4). Unvaccinated mice were challenged as described with the Gamma strain. At day 4 mice were euthanized and an immunohistochemical analysis of N expression was performed on brain samples. FIG. 21C shows expression of the SARS-CoV2 Nucleocapsid protein in mice brain (Day 6). Unvaccinated mice were challenged as described with the Gamma strain. At day 6 mice were euthanized due to their health condition and an immunohistochemical analysis of N expression was performed on brain samples. FIG. 21D shows expression of the SARS-CoV2 Nucleocapsid protein in mice brain (Day 6). Mice vaccinated with CoroVaxG.3-P were challenged as described with the Gamma strain. At day 6 mice were euthanized and an immunohistochemical analysis of N expression was performed on brain samples. Negative staining of the Nucleocapsid was observed at day 4 as well.

### DEFINITIONS

To facilitate an understanding of the present technology, a number of terms and phrases are defined below. Additional definitions are set forth throughout the detailed description.

The terms “host” or “subject,” as used herein, refer to an individual to be treated by (e.g., administered (e.g., injectably administered)) compositions and methods of the present disclosure. Subjects include, but are not limited to, mammals (e.g., murines, simians, equines, bovines, porcines, canines, felines, and the like), and most preferably includes humans. In the context of the disclosure, the term “subject” generally refers to an individual who will be administered (e.g., injectably and/or intranasal administered) or who has been administered one or more compositions of the present disclosure.

In some embodiments, the subject is at elevated risk for infection (e.g., by a coronavirus). In some embodiments, the subject may have a healthy or normal immune system. In some embodiments, the subject is one that has a greater than normal risk of being exposed to a pathogen (e.g., a coronavirus). In some embodiments, the subject is a soldier, an emergency responder or other subject that has a higher than normal risk of being exposed to a pathogen (e.g., a coronavirus).

As used herein, the terms “at risk for infection” and “at risk for disease” refer to a subject that is predisposed to experiencing a particular infection or disease (e.g., a coronavirus). This predisposition may be genetic, or due to other factors (e.g., age, immunosuppression, compromised immune system, immunodeficiency, environmental conditions, exposures to detrimental compounds present in the environment, etc.). Thus, it is not intended that embodiments of the present disclosure be limited to any particular risk (e.g., a subject may be “at risk for disease” simply by being exposed to and interacting with other people), nor is it intended that embodiments of the present disclosure be limited to any particular disease.

A used herein, the term “immune response” and grammatical equivalents thereof refer to a response by the immune system of a subject. For example, immune responses include, but are not limited to, a detectable alteration (e.g., increase) in Toll-like receptor (TLR) activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system), and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the term “immunity” refers to protection from disease (e.g., preventing or attenuating (e.g., suppression) of a sign, symptom or condition of the disease) upon exposure to a microorganism (e.g., a virus) capable of causing the disease. Immunity can be innate (e.g., non-adaptive (e.g., non-acquired) immune responses that exist in the absence of a previous exposure to an antigen) and/or acquired/adaptive (e.g., immune responses that are mediated by B and T cells following a previous exposure to antigen (e.g., that exhibit increased specificity and reactivity to the antigen)).

As used herein, the term “antibody” refers to an immunoglobulin molecule that is typically composed of two identical pairs of polypeptide chains, each pair having one “light” (L) chain and one “heavy” (H) chain. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. Each heavy chain is comprised of a heavy chain variable region (abbreviated herein as HCVR or V_{H}) and a heavy chain constant region. The heavy chain constant region is comprised of three domains, C_{H1}, C_{H2} and C_{H3}. Each light chain is comprised of a light chain variable region (abbreviated herein as LCVR or V_{L}) and a light chain constant region. The light chain constant region is comprised of one domain, C_{L}. The constant regions of the antibodies may mediate the binding of the immunoglobulin to host tissues or factors, including various cells of the immune system (e.g., effector cells) and the first component (C1q) of the classical complement system. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of each heavy/light chain pair (V_{H} and V_{L}), respectively, form the antibody binding site. The term “antibody” encompasses an antibody that is part of an antibody multimer (a multimeric form of antibodies), such as dimers, trimers, or higher-order multimers of monomeric antibodies. It also encompasses an antibody that is linked or attached to, or otherwise physically or functionally associated with, a non-antibody moiety. Further, the term “antibody” is not limited by any particular method of producing the antibody. For example, it includes, inter alia, recombinant antibodies, synthetic antibodies, monoclonal antibodies, polyclonal antibodies, bi-specific antibodies, and multi-specific antibodies.

The terms “fragment of an antibody,” “antibody fragment,” and “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). An antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H}1 domains, (ii) a F(a′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

In the context of the present disclosure, a “neutralizing antibody” is an antibody that binds to a virus (e.g., a coronavirus) and interferes with the virus' ability to infect a host cell.

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the terms “immunogen” and “antigen” are used interchangeably to refer to an agent (e.g., a microorganism (e.g., bacterium, virus, or fungus) and/or portion or component thereof (e.g., protein, glycoprotein, lipoprotein, peptide, glycopeptide, lipopeptide, toxoid, carbohydrate, tumor-specific antigen, etc.)) that is capable of eliciting an immune response in a subject. By “epitope” is meant a sequence of an antigen that is recognized by an antibody or an antigen receptor. Epitopes also are referred to in the art as “antigenic determinants.” In certain embodiments, an epitope is a region of an antigen that is specifically bound by an antibody or a T cell receptor. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The immunogen or antigen can be a protein or peptide of viral, bacterial, parasitic, fungal, protozoan, prion, cellular, or extracellular origin, which provokes an immune response in a mammal, preferably leading to protective immunity. An immunogen or antigen also may be based on one or more antigenic components of a particular organism and can be generated using recombinant DNA technology.

The term “vaccine,” as used herein, refers to a biological preparation that stimulates a subject's immune system against a particular infectious agent and provides active acquired immunity to a particular infectious disease. A vaccine typically contains an agent that resembles a disease-causing microorganism and is often made from weakened or killed forms of the microbe, its toxins, or one of its surface proteins. The agent stimulates a subject's immune system to recognize the agent as a threat, destroy it, and to further recognize and destroy any of the microorganisms associated with that agent that it may encounter in the future. Vaccines can be prophylactic (to prevent or ameliorate the effects of a future infection by a pathogen), or therapeutic (to ameliorate a disease that has already occurred, such as cancer). There are several types of vaccines known and used in the art, including, for example, inactivated virus vaccines, live-attenuated virus vaccines, messenger RNA (mRNA) vaccines, subunit vaccines, recombinant vaccines, polysaccharide vaccines, conjugate vaccines, toxoid vaccines, and viral vector vaccines. The administration of vaccines is referred to as “vaccination.”

“Pseudotyping” refers to the process of producing viruses or viral vectors using foreign viral envelope proteins. The resulting virus is referred to as a “pseudotyped virus.” In some cases, the inability to produce viral envelope proteins renders the pseudovirus replication-incompetent, which enables investigation of dangerous viruses in a lower risk setting. Pseudotyping viral systems have been widely employed to study highly infectious and pathogenic viruses, such as Ebola virus, Middle Eastern Respiratory Syndrome (MERS) virus, or SARS viruses (McWilliams et al., Cell Rep. (2019) 26:1718-26.e4. doi: 10.1016/j.celmp.2019.01.069; Liu et al., Antiviral Res. (2018) 150:30-8. doi: 10.1016/j.antiviral.2017.12.007; and Fukushi et al., SARS- and Other Coronaviruses: Laboratory Protocols. Totowa, NJ: Humana Press (2008). p. 331-8). The two most commonly used pseudotyping systems are retro/lentiviruses and vesicular stomatitis virus (VSV) which lacks the VSV envelope glycoprotein (VSVΔG). The use of replication-restricted pseudoviruses bearing foreign viral coat proteins represents a safe and useful method that has been widely adopted by virologists to study viral entry, detection of neutralizing antibodies in serum samples, and therapeutic development under less stringent biosafety conditions (e.g., biosafety level-2 (BSL-2)). Pseudotyped viruses have been used to produce vaccine candidates against HIV (Racine et al., AIDS Research and Therapy. 14 (1): 55. doi:10.1186/s12981-017-0179-2); Nipah henipavirus (Nie et al., Emerging Microbes & Infections. 8 (1): 272-281; doi:10.1080/22221751.2019.1571871); Rabies lyssavirus (Moeschler et al., Viruses. 8 (9): 254. doi:10.3390/v8090254), SARS-CoV (Kapadia et al., Virology. 376(1): 165-172. doi:10.1016/j.virol.2008.03.002); Zaire ebolavirus (Salata et al., Viruses. 11 (3): 274. doi:10.3390/v11030274), and SARS-CoV-2 (Johnson et al., Journal of Virology. 94 (21). doi:10.1128/JVI.01062-20; and Condor Capcha et al., Front. Cardiovasc. Med., 15 January (2021)).

### DETAILED DESCRIPTION

The present disclosure provides compositions and methods for inducing an immune response against one or more viruses in a subject. In some embodiments, the one or more viruses are one or more emerging viruses. The terms “emerging virus” or “emergent virus,” as used herein, refer to a newly discovered virus, one that is increasing in incidence or geographic range over the last two decades, or has the potential to increase in incidence. The term “reemerging virus,” as used herein, refers to a mutant or variant of a known virus that causes new epidemics with considerable virulence, or a virus whose incidence has increased after significant decline. Examples of emerging viruses include, but are not limited to, filoviruses (Ebola, Marburg), henipaviruses (Nipah virus, Hendra virus), Lassa virus, Lujo virus, South American hemorrhagic fever viruses (Junin, Machupo, Guanarito, Chapare, Sabia), Crimean-Congo hemorrhagic fever virus, Rift Valley fever virus (RVFV), hantaviruses, Dengue virus, West Nile virus, SARS coronavirus (e.g., SARS-CoV, SARS-CoV-2), MERS coronavirus, tick-borne encephalitis viruses, chikungunya virus, and Zika virus.

In some embodiments, the disclosure provides a chimeric, replication incompetent adenoviral vector. In some embodiments, the disclosure provides a chimeric, replication incompetent adenoviral vector comprising (a) a hybrid promoter comprising an exogenous intron, (b) a nucleic acid sequence encoding a viral antigen operatively linked to the hybrid promoter, (c) a post-transcriptional regulatory element; and (d) a modified fiber protein. Also provided are compositions comprising the adenoviral vector and methods of using same to induce an immune response against a virus, such as an emerging virus (e.g., SARS-CoV-2) in a subject.

### Adenoviral Vectors

Adenovirus is a medium-sized (90-100 nm), nonenveloped icosahedral virus containing approximately 36 kilobases (kb) of double-stranded DNA. The term “adenovirus,” as used herein, refers to an adenovirus that retains the ability to participate in the adenovirus life cycle and has not been physically inactivated by, for example, disruption (e.g., sonication), denaturing (e.g., using heat or solvents), or cross-linkage (e.g., via formalin cross-linking). The “adenovirus life cycle” includes (1) virus binding and entry into cells, (2) transcription of the adenoviral genome and translation of adenovirus proteins, (3) replication of the adenoviral genome, and (4) viral particle assembly (see, e.g., Fields Virology, 5th ed., Knipe et al. (eds.), Lippincott Williams & Wilkins, Philadelphia, Pa. (2006)). The term “adenoviral vector,” as used herein, refers to an adenovirus in which the adenoviral genome has been manipulated to accommodate a nucleic acid sequence that is non-native with respect to the adenoviral genome. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

Several features of adenoviruses make them ideal vehicles for transferring genetic material to cells for therapeutic applications (e.g., gene therapy, immunotherapy, or as vaccines). For example, adenoviruses can be produced in high titers (e.g., about 10¹³ particle units (pu)), and can transfer genetic material to nonreplicating and replicating cells. The adenoviral genome can be manipulated to carry a large amount of exogenous DNA (up to about 8 kb), and the adenoviral capsid can potentiate the transfer of even longer sequences (Curiel et al., Hum. Gene Ther., 3: 147-154 (1992)). Additionally, adenoviruses generally do not integrate into the host cell chromosome, but rather are maintained as a linear episome, thereby minimizing the likelihood that a recombinant adenovirus will interfere with normal cell function.

The adenovirus capsid mediates the key interactions of the early stages of the infection of a cell by the virus and is required for packaging adenovirus genomes at the end of the adenovirus life cycle. The capsid comprises 252 capsomeres, which includes 240 hexon trimers, 12 penton base pentamer proteins, and 12 trimer fibers (Ginsberg et al., Virology, 28: 782-83 (1966)). The hexon comprises three identical proteins, namely polypeptide II (Roberts et al., Science, 232: 1148-51 (1986)). The penton base comprises five identical proteins and the fiber comprises three identical proteins. Proteins IIIa, VI, and IX are present in the adenoviral coat and are believed to stabilize the viral capsid (Stewart et al., Cell, 67: 145-54 (1991), and Stewart et al., EMBO J., 12(7): 2589-99 (1993)). The expression of the capsid proteins, with the exception of pIX, is dependent on the adenovirus polymerase protein. Therefore, major components of an adenovirus particle are expressed from the genome only when the polymerase protein gene is present and expressed.

The adenoviral vector may be of any serotype or combination of serotypes. Over 50 serotypes of adenovirus have been identified, which are classified as subgroup A (e.g., serotypes 12, 18, and 31), subgroup B (e.g., serotypes 3, 7, 11, 14, 16, 21, 34, 35, 50, and 55), subgroup C (e.g., serotypes 1, 2, 5, and 6), subgroup D (e.g., serotypes 8, 9, 10, 13, 15, 17, 19, 20, 22-30, 32, 33, 36-39, and 42-49, 51, 53, 54, 56), subgroup E (e.g., serotype 4), subgroup F (e.g., serotypes 40 and 41), subgroup G (e.g., serotype 52). Various serotypes of adenovirus are available from the American Type Culture Collection (ATCC, Manassas, Va.). In one embodiment, the adenovirus or adenoviral vector is a serotype 5 adenovirus or adenoviral vector (“Ad5”).

In some embodiments, the adenoviral vector is chimeric. A “chimeric” adenovirus or adenoviral vector may comprise an adenoviral genome that is derived from two or more (e.g., 2, 3, 4, etc.) different adenovirus serotypes. In some embodiments, a chimeric adenovirus or adenoviral vector can comprise approximately equal amounts of the genome of each of the two or more different adenovirus serotypes. When the chimeric adenoviral vector genome is comprised of the genomes of two different adenovirus serotypes, the chimeric adenoviral vector genome preferably comprises no more than about 95% (e.g., no more than about 90%, about 85%, about 80%, about 75%, about 70%, about 65%, about 60%, about 55%, about 50%, about 45%, or about 40%) of the genome of one of the adenovirus serotypes, with the remainder of the chimeric adenovirus genome being obtained or derived from the genome of the other adenovirus serotype. In one embodiment, the majority (i.e., greater than 50%) of the genome of the adenovirus or adenoviral vector is obtained or derived from a serotype 5 adenovirus. In some embodiments, the adenoviral vector is “chimeric” in that a majority of the genome is derived from a first serotype adenovirus (e.g. serotype 5 adenovirus), and the adenoviral vector comprises a nucleotide sequence encoding a modified fiber protein, wherein the modified fiber protein comprises one or more fiber protein domains from the first serotype adenovirus (e.g. the serotype 5 adenovirus) and a fiber knob domain from a second serotype adenovirus (e.g. a serotype 3 adenovirus).

The adenoviral vector can be replication-deficient or conditionally replication-competent. An adenoviral vector that is “replication-competent” can replicate in typical host cells, i.e., cells typically capable of being infected by an adenovirus. In contrast, a “replication-deficient” or “replication-incompetent” adenovirus or adenoviral vector requires complementation of one or more gene functions or regions of the adenoviral genome that are required for replication, as a result of, for example, a deficiency in one or more replication-essential gene function or regions, such that the adenovirus or adenoviral vector does not replicate in typical host cells, especially those in a human to be infected by the adenovirus or adenoviral vector.

A “conditionally-replicating” adenovirus or adenoviral vector is an adenovirus or adenoviral vector that has been engineered to replicate under pre-determined conditions. For example, replication-essential gene functions, e.g., gene functions encoded by the adenoviral early regions, can be operably linked to an inducible, repressible, or tissue-specific promoter. In such embodiments, replication requires the presence or absence of specific factors that activate or repress the promoter. Conditionally-replicating adenoviral vectors are further described in, e.g., U.S. Pat. Nos. 5,998,205 and 6,824,771.

A deficiency in a gene function or genomic region, as used herein, is defined as a disruption (e.g., deletion) of sufficient genetic material of the adenoviral genome to obliterate or impair the function of the gene (e.g., such that the function of the gene product is reduced by at least about 2-fold, 5-fold, 10-fold, 20-fold, 30-fold, or 50-fold) whose nucleic acid sequence was disrupted (e.g., deleted) in whole or in part. Deletion of an entire gene region often is not required for disruption of a replication-essential gene function. However, for the purpose of providing sufficient space in the adenoviral genome for one or more transgenes, removal of a majority of one or more gene regions may be desirable. While deletion of genetic material is preferred, mutation of genetic material by addition or substitution also is appropriate for disrupting gene function. Replication-essential gene functions are those gene functions that are required for adenovirus replication (e.g., propagation) and are encoded by, for example, the adenoviral early regions (e.g., the E1, E2, and E4 regions), late regions (e.g., the L1, L2, L3, L4, and L5 regions), genes involved in viral packaging (e.g., the IVa2 gene), and virus-associated RNAs (e.g., VA-RNA-1 and/or VA-RNA-2).

The early region 1A and 1B (E1A and E1B) genes encode proteins required for a productive adenovirus lytic cycle (Fields, supra). E1A is the first viral gene transcribed after infection and produces two related proteins, 243R and 289R, which induce transcription of the other early viral gene regions and stimulate infected cells to enter S-phase of the cell cycle. The E1B region encodes two major proteins, E1B19K and E1B55K. The E1B55K protein binds the cellular tumor suppressor p53 and can block p53-mediated apoptosis and inhibition of viral and cellular replication. The E1B19K protein is a Bcl-2 homologue that interacts with Bax and inhibits apoptosis, allowing the virus to replicate longer (Sundararajan, R. and White, E, J. Virology, 75:7506-7516 (2001)). It has recently been demonstrated that the E1B proteins may not be essential for replication of oncolytic adenoviruses (Lopez et al., Mol. Ther., 20: 2222-2233 (2012); and Viale et al., J. Invest. Dermatol., 133(11):2576-2584 (2013)). The E1A proteins have been shown to induce S-phase in infected cells by associating with p300/CBP or the retinoblastoma (Rb) protein (Howe et al., Proc. Natl. Acad. Sci. USA, 87: 5883-5887 (1990); Wang et al., Mol. Cell. Biol., 11: 4253-4265 (1991); Howe, J. A. and Bayley, S. T. Virology, 186: 15-24 (1992)). Rb and p300 regulate the activity of E2F transcription factors, which coordinate the expression of cellular genes required for cell cycle progression (Helin, K., Curr. Opin. Genet. Dev., 8: 28-35 (1998)). Thus, E1A gene products play a role in viral genome replication by driving entry of quiescent cells into the cell cycle, in part, by displacing E2F transcription factors from the retinoblastoma protein (pRb) tumor suppressor (Liu, X. and Marmorstein, R., Genes & Dev., 21: 2711-2716 (2007)).

In some embodiments, the adenoviral vector may comprise a deletion, in whole or in part, of one or more regions of the adenoviral genome. In some embodiments, the adenoviral vector comprises a deletion of sufficient genetic material of the adenoviral genome to obliterate or impair the function of the gene (e.g., such that the function of the gene product is reduced by at least about 2-fold, 5-fold, 10-fold, 20-fold, 30-fold, or 50-fold) whose nucleic acid sequence was disrupted (e.g., deleted) in whole or in part. For the purpose of providing sufficient space in the adenoviral genome for one or more non-native nucleic acid sequences (or “transgenes”), removal of a majority of one or more gene regions may be desirable. In this regard, the adenovirus or adenoviral vector may comprise a deletion of all or part of any of the adenoviral early regions (e.g., E1, E2, E3 and E4 regions), the late regions (e.g., the L1, L2, L3, L4, and L5 regions), genes involved in viral packaging (e.g., the IVa2 gene), and/or virus-associated RNAs (e.g., VA-RNA-1 and/or VA-RNA-2). In one embodiment, the adenovirus or adenoviral vector comprises a deletion of all or part of the E1A region, a deletion of all or part of the E1B region of the adenoviral genome, a deletion of all or part of the E3 region of the adenoviral genome, and/or a deletion of all or part of the E4 region of the adenoviral genome. The size of the deletion may be tailored so as to retain an adenovirus or adenoviral vector whose genome closely matches the optimum genome packaging size. A larger deletion will accommodate the insertion of larger non-native nucleic acid sequences in the adenovirus or adenoviral genome.

By removing all or part of certain regions of the adenoviral genome, for example, the E1B, E3, and/or E4 regions of the adenoviral genome, the resulting adenoviral vector is able to accept inserts of exogenous non-native nucleic acid sequences while retaining the ability to be packaged into adenoviral capsids. Thus, in another embodiment, the adenovirus or adenoviral vector comprises one or more non-native nucleic acid sequences. A non-native nucleic acid sequence can be inserted at any position in the adenoviral genome so long as insertion allows for the formation of adenovirus or the adenoviral vector particle. A “non-native” nucleic acid sequence is any nucleic acid sequence (e.g., DNA, RNA, or cDNA sequence) that is not a naturally occurring nucleic acid sequence of an adenovirus in a naturally occurring position. The terms “non-native nucleic acid sequence,” “heterologous nucleic acid sequence,” and “exogenous nucleic acid sequence” are synonymous and can be used interchangeably in the context of the present disclosure. The non-native nucleic acid sequence preferably is DNA and preferably encodes a protein (e.g., one or more nucleic acid sequences encoding one or more proteins). The term “transgene” is defined herein as a non-native nucleic acid sequence that is operably linked to appropriate regulatory elements (e.g., a promoter), such that the non-native nucleic acid sequence can be expressed to produce an RNA or protein (e.g., a regulatory RNA sequence, peptide, or polypeptide). The regulatory elements (e.g., promoter) can be native or non-native to the adenovirus or adenoviral vector.

Replication-deficient adenoviral vectors can be produced in complementing cell lines that provide gene functions not present in the replication-deficient adenoviral vector, but required for viral propagation, at appropriate levels in order to generate high titers of viral vector stock. Such complementing cell lines are known and include, but are not limited to, 293 cells (described in, e.g., Graham et al., J. Gen. Virol., 36: 59-72 (1977)), PER.C6 cells (described in, e.g., International Patent Application Publication WO 1997/000326, and U.S. Pat. Nos. 5,994,128 and 6,033,908), and 293-ORF6 cells (described in, e.g., International Patent Application Publication WO 95/34671 and Brough et al., J. Virol., 71: 9206-9213 (1997)). Additional suitable complementing cells are described in, for example, U.S. Pat. Nos. 6,677,156 and 6,682,929, and International Patent Application Publication WO 2003/020879. In some instances, one or more replication-essential gene functions lacking in a replication-deficient adenoviral vector can be supplied by a helper virus, e.g., an adenoviral vector that supplies in trans one or more essential gene functions required for replication of the replication-deficient adenoviral vector. Methods for the production and purification of adenoviruses and adenoviral vectors are described in, e.g., U.S. Pat. No. 6,194,191, and International Patent Application Publications WO 99/54441, WO 98/22588, WO 98/00524, WO 96/27677, and WO 2003/078592.

### Hybrid Promoter

The adenoviral vector described herein desirably comprises a hybrid promoter. As used herein, the term “promoter” refers to a DNA sequence that directs the binding of RNA polymerase, thereby promoting RNA synthesis. A nucleic acid sequence is “operably linked” or “operatively linked” to a promoter when the promoter is capable of directing transcription of that nucleic acid sequence. A promoter can be native or non-native to the nucleic acid sequence to which it is operably or operatively linked. Techniques for operably linking sequences together are well known in the art.

In the context of the present disclosure, the promoter is “hybrid” in that it comprises promoter elements (e.g., promoters, enhancers, polyadenylation signals, transcription terminators, internal ribosome entry sites (IRES)) obtained or derived from two or more different sources. In some embodiments, the hybrid promoter comprises an exogenous intron. The term “exogenous intron” refers to an intron derived from a source other than the adenovirus.

In some embodiments, the hybrid promoter comprises a cytomegalovirus (CMV) immediate early enhancer, a β-actin promoter, and an exogenous intron. The CMV major immediate early (MIE) promoter is a complex promoter, containing an enhancer (−520 to −65 nucleotides, nt), a unique region (−780 to −610 nt), and a modulator (−1145 to −750 nt) in addition to the core promoter (−65 to +3 nt). The 800-bp CMV immediate early enhancer/promoter is widely used in the art to achieve rapid and ubiquitous expression in gene transfer applications. The chicken beta actin (CBA) promoter is typically utilized in gene transfer applications as a hybrid promoter with the CMV immediate-early enhancer region, and intron 1/exon 1 of the chicken beta actin gene (commonly called the CAGGS promoter; Niwa et al., Gene, 1991; 108:193-199) to provide ubiquitous and long-term gene expression in a variety of cell types. The nucleic acid sequence of the chicken beta actin gene, including the promoter sequence, is available from the National Center of Biotechnology Information (NBCI) under Gene ID: 396526. Elements of the chicken beta actin promoter also are described in, e.g., Fregien, N. and Davidson, N., Gene, 48(1): 1-11 (1986).

In some embodiments, the exogenous intron is a chimeric intron. As used herein, the term “chimeric intron” refers an intron having sequences from two or more different sources. The borders between introns and exons are marked by specific nucleic acid sequences within a pre-mRNA, which delineate where splicing will occur. Such boundaries are referred to herein as “splice sites.” The term “splice site,” as used herein, refers to polynucleotides that are capable of being recognized by the spicing machinery of a eukaryotic cell as suitable for being cut and/or ligated to another splice site. Splice sites allow for the excision of introns present in a pre-mRNA transcript. Typically, the 5′ splice boundary is referred to as the “splice donor site” or the “5′ splice site,” and the 3′ splice boundary is referred to as the “splice acceptor site” or the “3′ splice site.” In some embodiments, a chimeric intron comprises a nucleic acid encoding a splice donor site from a first source (e.g., organism or species) and a splice acceptor site from a second source (e.g., organism or species). A chimeric intron also may comprise one or more transcriptional regulatory elements and/or enhancer sequences. In some embodiments, a chimeric intron is positioned between an exon of a hybrid promoter and transgene. In the context of the present disclosure, the chimeric intron may comprise any suitable splice donor site and any suitable splice acceptor site. In some embodiments, the chimeric intron comprises a splice donor site from a β-actin gene. In some embodiments, the chimeric intron comprises a splice acceptor site from a parvovirus. In some embodiments, the chimeric intron comprises a splice donor suite from a β-actin gene and a splice acceptor site from a parvovirus. Parvoviruses are a family of viruses having linear, single-stranded DNA genomes. The parvovirus genome typically contains two genes, termed the NS/rep gene and the VP/cap gene. The NS gene encodes the non-structural protein NS1, which is the replication initiator protein, and the VP gene encodes the viral protein that the viral capsid is made of. Exemplary parvoviruses subfamilies include the Amdoparvovirus, Artiparvovirus, Aveparvovirus, Bocaparvovirus, Copiparvovirus, Dependoparvovirus, Erythroparvovirus, Loriparvovirus, Protoparvovirus, and Tetraparvovirus subfamilies. In some embodiments, the parvovirus is a protoparvovirus. Exemplary protopavoviruses include canine parvovirus, feline parvovirus, Kilham rat virus, minute virus of mice, mouse parvovirus, tumor virus X, rat minute virus, rate parvovirus 1, and porcine parvovirus. In some embodiments, the chimeric intron comprises a splice acceptor site is from minute virus of mice. In some embodiments, the chimeric intron comprises a splice donor site from a β-actin gene and a splice acceptor site from the minute virus of mice (MVM). The splice site junctions of β-actin genes from several species have been identified (see, e.g., Sadusky et al., Current Biology, 14(6): 505-509 (2004); D. Bhattacharya, K. Weber, Curr. Genet., 31 (1997), pp. 439-446; and P. Sheterline, J. Clayton, J. C. Sparrow, Protein Profile Actin, Fourth Edition, P. Sheterline, Oxford University Press, Oxford (1998)). Minute virus of mice (MVM) is a member of the Parvovirus genus, and includes a family of small, nonenveloped, single-stranded DNA viruses. They depend on active division of their host cells for their own multiplication but do not require coinfection with other viruses to replicate. The splice junctions of MVM mRNAs have been mapped (see, e.g., Jongeneel et al., J. Virol., 59(3): 564-573 (1986)).

The disclosed adenoviral vector desirably comprises a post-transcriptional regulatory element. The term “posttranscriptional response element,” as used herein, refers to a nucleic acid sequence that, when transcribed, adopts a tertiary structure that enhances expression of a gene. Examples of posttranscriptional regulatory elements include, but are not limited to, woodchuck hepatitis virus posttranscriptional regulatory element (WPRE), mouse RNA transport element (RTE), constitutive transport element (CTE) of the simian retrovirus type 1 (SRV-1), the CTE from the Mason-Pfizer monkey virus (MPMV), and the 5′ untranslated region of the human heat shock protein 70 (Hsp70 5′UTR). In some embodiments, the adenoviral vector comprises a woodchuck hepatitis virus posttranscriptional regulatory element (WPRE). The WPRE is characterized in, e.g., Donello et al., J Virol 1998; 72: 5085-5092; and Hope T., Curr Top Microbiol Immunol 2002; 261: 179-189.

Exemplary hybrid promoter nucleic acid sequences for use in the adenoviral vector described herein include SEQ ID NO: 1, SEQ ID NO: 2, SEQ ID NO: 3, and SEQ ID NO: 4. In some embodiments, the hybrid promoter may comprise a nucleic acid sequence that is at least about 70% identical (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO: 1, SEQ ID NO: 2, SEQ ID NO: 3, or SEQ ID NO: 4. In some embodiments, the hybrid promoter comprises a nucleic acid sequence having at least 80% sequence identity to SEQ ID NO: 1, SEQ ID NO: 1, SEQ ID NO: 3, or SEQ ID NO: 4. In some embodiments, the hybrid promoter comprises a nucleic acid sequence having at least 90% sequence identity to SEQ ID NO: 1, SEQ ID NO: 1, SEQ ID NO: 3, or SEQ ID NO: 4. In some embodiments, the hybrid promoter comprises the nucleic acid sequence of SEQ ID NO: 1, SEQ ID NO: 1, SEQ ID NO: 3, or SEQ ID NO: 4. The degree of nucleic acid and/or amino acid identity can be determined using any method known in the art, such as the BLAST sequence database.

### Viral Antigens

In some embodiments, the adenoviral vector further comprises at least one nucleic acid sequence encoding at least one viral antigen. In some embodiments, the adenoviral vector further comprises a nucleic acid sequence encoding a viral antigen is operably linked to the hybrid promoter. In some embodiments, the adenoviral vector further comprises at least one non-native nucleic acid sequence encoding an emerging virus antigen operatively linked to the hybrid promoter. As discussed above, the viral antigen may be obtained or derived from any suitable virus, including emerging viruses such as filoviruses (Ebola, Marburg), henipaviruses (Nipah virus, Hendra virus), Lassa virus, Lujo virus, South American hemorrhagic fever viruses (Junin, Machupo, Guanarito, Chapare, Sabia), Crimean-Congo hemorrhagic fever virus, Rift Valley fever virus (RVFV), hantaviruses, Dengue virus, West Nile virus, SARS coronavirus (e.g., SARS-CoV, SARS-CoV-2), MERS coronavirus, tick-borne encephalitis viruses, chikungunya virus, and Zika virus. The virus, however, is not limited to these particular examples, and the present disclosure encompasses antigens obtained or derived from viruses yet to be identified or classified as “emerging.”

Nucleic acid and amino acid sequences of numerous viral antigens that may be incorporated into the disclosed adenoviral vector are known in the art. In this regard, exemplary Zika virus antigens are described in, e.g., Pattnaik et al., Vaccines (Basel). 2020 June; 8(2): 266. Exemplary Ebola virus antigens are described in, e.g., Runa et al., MOJ Proteomics Bioinform. 2018; 7(1):1-7. DOI: 10.15406/mojpb.2018.07.00205. Exemplary Chikungunya virus antigens are described in, e.g., Gao et al., Front Microbiol. 2019; 10: 2881. Exemplary Nipah virus antigens are described in, e.g., Loomis et al., Front. Immunol., 11 Jun. 2020; doi: 10.3389/fimmu.2020.00842. Exemplary Lassa virus antigens are described in, e.g., Sayed et al., International Journal of Peptide Research and Therapeutics, 26: 2089-2107 (2020). Exemplary Rift Valley fever virus antigens are described in, e.g., Chrun et al., NPJ Vaccines. 2018; 3: 14. Exemplary dengue virus antigens are described in, e.g., Deng et al., Vaccines (Basel). 2020 March; 8(1): 63. Exemplary West Nile virus antigens are described in, e.g., Sebastian Ulbert (2019) Human Vaccines & Immunotherapeutics, 15:10, 2337-2342, DOI: 10.1080/21645515.2019.1621149. Exemplary MERS coronavirus antigens are described in, e.g., Du et al., Expert Review of Vaccines, 6 Apr. 2016, 15(9):1123-1134. Emerging viruses are further described in, e.g., Howley et al. (eds.), Fields Virology: Emerging Viruses 7th Edition (2020).

In some embodiments, the viral antigen is obtained or derived from a coronavirus. Coronaviruses are a large family of viruses that are common in people and many different species of animals, including camels, cattle, cats, and bats. Rarely, animal coronaviruses can infect humans and then spread between humans, such as with MERS-CoV, SARS-CoV, and SARS-CoV-2 (COVID-19). Coronaviruses are named for the crown-like spikes on their surface. There are four main sub-groupings of coronaviruses, known as alpha, beta, gamma, and delta. Human coronaviruses were first identified in the mid-1960s. Seven coronaviruses have been identified that can infect humans: 229E (alpha coronavirus) NL63 (alpha coronavirus); OC43 (beta coronavirus); HKU1 (beta coronavirus); MERS-CoV (the beta coronavirus that causes Middle East Respiratory Syndrome, or MERS); SARS-CoV (the beta coronavirus that causes severe acute respiratory syndrome, or SARS); and SARS-CoV-2 (the novel coronavirus that causes coronavirus disease 2019, or COVID-19). In some embodiments, the coronavirus antigen is obtained or derived from a betacoronavirus. The SARS-CoV-2 virus, MERS-CoV, and SARS-CoV are examples of betacoronaviruses. All three of these viruses have their origins in bats. MERS-CoV and SARS-CoV have been known to cause severe illness in people. The complete clinical picture with regard to COVID-19 is not fully understood. Reported illnesses have ranged from mild to severe, including illness resulting in death. Older people and people with certain underlying health conditions like heart disease, lung disease and diabetes, for example, seem to be at greater risk of serious illness.

The disclosed vectors and methods may induce an immune response against any coronavirus, including coronaviruses that infect humans and non-human mammals (e.g., canines or felines), such as, for example, coronavirus OC43, coronavirus 229E, coronavirus NL63, coronavirus HKU1, MERS-CoV, SARS-CoV, or SARS-CoV-2 (COVID-19). In some embodiments, the disclosed adenoviral vectors and methods may be used to induce an immune response against all coronaviruses (also referred to as “pan-coronavirus” or “universal coronavirus” vaccines). In such cases, the adenoviral vector may encode one coronavirus antigen or a plurality of coronavirus antigens that induce protective immune responses against all coronaviruses, both known and unknown.

In some embodiments, the disclosed vectors and methods induce an immune response against SARS-CoV-2. SARS-CoV-2 is a monopartite, single-stranded, and positive-sense RNA virus with a genome size of 29,903 nucleotides, making it the second-largest known RNA genome. The virus genome consists of two untranslated regions (UTRs) at the 5′ and 3′ ends and 11 open reading frames (ORFs) that encode 27 proteins. The first ORF (ORF1/ab) constitutes about two-thirds of the virus genome, encoding 16 non-structural proteins (NSPs), while the remaining third of the genome encodes four structural proteins and at least six accessory proteins. The structural proteins are spike glycoprotein (S), membrane protein (M), envelope protein (E), and nucleocapsid protein (N), while the accessory proteins are orf3a, orf6, orf7a, orf7b, orf8, and orf10 (Wu et al., Cell Host Microbe, 27: 325-328 (2020); Chan et al., Emerg. Microbes Infect., 9: 221-236 (2020); Chen et al., Lancet, 395: 507-513 (2020); and Ceraolo, C.; Giorgi, F. M, J. Med. Virol., 92, 522-528 (2020)). Of the NSPs, (1) NSP1 suppresses the antiviral host response, (2) NSP3 is a papain-like protease, (3) NSP5 is a 3CLpro (3C-like protease domain), (4) NSP7 makes a complex with NSP8 to form a primase, (5) NSP9 is responsible for RNA/DNA binding activity, (6) NSP12 is an RNA-dependent RNA polymerase (RdRp), (7) NSP13 is confirmed as a helicase, (8) NSP14 is a 3′-5′ exonuclease (ExoN), (9) NSP15 is a poly(U)-specific endoribonuclease (XendoU). The remaining NSPs are involved in transcription and replication of the viral genome (Chan et al., Emerg. Microbes Infect., 9: 221-236 (2020); and Krichel et al., Biochem. J., 477: 1009-1019 (2019)).

Coronavirus spike proteins are known to elicit potent neutralizing-antibody and T-cell responses. The ability of a virus (e.g., coronavirus OC43, coronavirus 229E, coronavirus NL63, coronavirus HKU1, MERS-CoV, SARS-CoV, or SARS-CoV-2 (COVID-19)) to enter cells and establish infection is mediated by the interactions of its Spike glycoproteins with human cell surface receptors. In the case of coronaviruses, Spike proteins are large type I transmembrane protein trimers that protrude from the surface of coronavirus virions. Each Spike protein comprises a large ectodomain (comprising S1 and S2), a transmembrane anchor, and a short intracellular tail. The S1 subunit of the ectodomain mediates binding of the virion to host cell-surface receptors through its receptor-binding domain (RBD). The S2 subunit fuses with both host and viral membranes, by undergoing structural changes.

SARS-CoV-2 utilizes the Spike glycoprotein to interact with cellular receptor ACE2 (Zhou et al., Nature 579: 270-273, doi:10.1038/s41586-020-2012-7 (2020); Hoffmann et al., Cell, S0092-8674(0020)30229-30224, doi:10.1016/j.cell.2020.02.052 (2020) doi:10.1016/j.cell.2020.02.052 (2020)). The amino acid sequence of the SARS-CoV-2 spike protein has been deposited with the NCBI under Accession No. QHD43416. The nucleic acid sequence of the SARS-CoV-2 spike protein is:

(SEQ ID NO: 14)
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVENATRFASVYAWNRKRISN
CVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGST
PCNGVEGENCYFPLQSYGFQPTNGVGYQPYRVVVLSFELLHAPATVCGPKKSTNLVKN
KCVNFNFNGLTGTGVLTESNKKFLPFQQFGRDIADTTDAVRDPQTLEILDITPCSFGGVS
VITPGTNTSNQVAVLYQDVNCTEVPVAIHADQLTPTWRVYSTGSNVFQTRAGCLIGAEH
VNNSYECDIPIGAGICASYQTQTNSPRRARSVASQSIIAYTMSLGAENSVAYSNNSIAIPT
NFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGWTFGAGAALQIP
FAMQMAYRFNGIGVTQNVLYENQKLIANQFNSAIGKIQDSLSSTASALGKLQDVVNQN
AQALNTLVKQLSSNFGAISSVLNDILSRLDKVEAEVQIDRLITGRLQSLQTYVTQQLIRAA
EIRASANLAATKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNLN
ESLIDLQELGKYEQYIKWPWYIWLGFIAGLIAIVMVTIMLCCMTSCCSCLKGCCSCGSCC
KFDEDDSEPVLKGVKLHYT.

Binding with ACE2 triggers a cascade of cell membrane fusion events for viral entry. The high-resolution structure of SARS-CoV-2 RBD bound to the N-terminal peptidase domain of ACE2 has recently been determined, and the overall ACE2-binding mechanism is virtually the same between SARS-CoV-2 and SARS-CoV RBDs, indicating convergent ACE2-binding evolution between these two viruses (Gui et al., CellRes 27, 119-129, doi:10.1038/cr.2016.152 (2017); Song et al., PLoS Pathog 14, e1007236-e1007236, doi:10.1371/journal.ppat.1007236 (2018); Yuan et al., Nat Commun 8, 15092-15092, doi:10.1038/ncomms15092 (2017); and Wan et al., J Virol, JVI.00127-00120, doi:10.1128/JVI.00127-20 (2020)).

In some embodiments, the adenoviral vector comprises at least one nucleic acid sequence encoding one or more coronavirus antigens, or portions or epitopes thereof. In some embodiments, the coronavirus antigen is the SARS-CoV-2 spike protein (“S” protein as provided by, e.g., UniProtKB Accession Number P0DTC2) or the spike protein receptor-binding domain (RBD) (see, e.g., Wrapp (2020), Science 367: 1260-63; Walls (2020) Cell 180: 1-12). In some embodiments, the coronavirus antigen is a viral transcription and/or replication protein (e.g., replicase polyprotein 1a (R1a) or replicase polyprotein lab (R lab)). In some embodiments, the coronavirus antigen is a viral budding protein (e.g., protein 3a or envelope small membrane protein (E)). In some embodiments, the coronavirus antigen is a virus morphogenesis protein (e.g., membrane protein (M)). In some embodiments, the coronavirus antigen is non-structural protein 6 (NS6), protein 7a (NS7A), protein 7b (NS7B), non-structural protein 8 (NS8), or protein 9b (NS9B). In some embodiments, the coronavirus antigen is a viral genome packaging protein (e.g., nucleocapsid protein (N or NC)). In some embodiments, the coronavirus antigen is an uncharacterized protein.

In some embodiments, the coronavirus antigen may comprise a protein and/or a nucleic acid, or a portion thereof, from a genetic variant of the SARS-CoV-2 virus, e.g., a SARS-CoV-2 variant of interest (VOI), variant of concern (VOC), or variant of high consequence. In some embodiments, the variant is B.1.526, B.1.525, P.2, B.1.1.7 (also known as “alpha,” 201/501Y.V1, and VOC 202012/01), P.1 (also known as “gamma”), B.1.351 (also known as “beta” and 20H/501Y.V2), B.1.427 (also known as “epsilon”), B.1.429 (also known as “epsilon”), B.1.617, or B.1.617.2 (also known as “delta”). SARS-CoV-2 variants are further described in, e.g., Zhou et al., Nature (Feb. 26, 2021); Volz et al., Cell 2021; 184(64-75); Korber et al., Cell 2021; 182(812-7); Davies et al., MedRXiv 2021; Horby et al., New & Emerging Threats Advisory Group, Jan. 21, 2021; Emary et al., Lancet (Feb. 4, 2021); Fact Sheet For Health Care Providers Emergency Use Authorization (EUA) Of Regen-Cov (fda.gov); Wang P, Wang M, Yu J, et al. Increased Resistance of SARS-CoV-2 Variant P.1 to Antibody Neutralization. BioRxiv 2021; and Li et al., Innovation (NY). 2021 May 11; 100116. doi: 10.1016/j.xinn.2021.100116).

In other embodiments, a coronavirus antigen may be synthetically generated using structural and/or computational biology methodologies (also referred to as “structural vaccinology”). Structural vaccinology aims to identify protective B-cell epitopes on the antigens and optimizing the antigens in terms of stability, epitope presentation, ease of production, and safety. Structural vaccinology involves experimental methods like X-ray crystallography, electron microscopy and mass spectrometry, and computational methods like structural modeling, computational scaffold design, and epitope prediction (Saylor et al., Front. Immunol., 24 Feb. 2020; doi.org/10.3389/fimmu.2020.00283; and Liljeroos et al., J Immunol Res. 2015; 2015: 156241).

In some embodiments, the viral antigen is a coronavirus spike (S) protein having an amino acid sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to the amino acid sequence of SEQ ID NO: 14. In some embodiments, the viral antigen is a spike protein comprising an amino acid sequence having at least one mutation relative to SEQ ID NO: 14. In some embodiments, the spike protein comprises at least one mutation, at least two mutations, at least three mutations, at least four mutations, at least five mutations, at least 6 six mutations, at least 7 mutations, at least 8 mutations, at least 9 mutations, at least 10 mutations, or more than 10 mutations relative to SEQ ID NO: 14. In some embodiments, the at least one mutation comprises D614G. In some embodiments, the at least one mutation comprises D614G, K986P, and V987P. In some embodiments, the spike protein comprises at least one mutation selected from P681R and P681H. For example, in some embodiments the spike protein comprises a D614G mutation and at least one mutation selected from P681R and P681H.

In some embodiments, the spike protein further comprises one or more mutations in the furin cleavage domain. The furin cleavage domain is defined by amino acids 682 to 685 of SEQ ID NO: 14. In some embodiments, the one or more mutations in the furin cleavage domain are selected from R682G, R683S and R685S. In some embodiments, the spike protein comprises R682G, R683S and R685S mutations in the furin cleavage domain.

In some embodiments, the spike protein further comprises a K417T mutation or a K417N mutation relative to SEQ ID NO: 14.

In some embodiments, the one or more mutations relative to SEQ ID NO: 14 comprise each of D614G, K986P, V987P, R682G, R683S, and R685S.

In some embodiments, the spike protein comprises one or more mutations shown in Table 1. The mutations shown in Table 1 are all relative to SEQ ID NO: 14. In some embodiments, the spike protein comprises one or more of the following mutations relative to SEQ ID NO: 14: L18F, T20N, P26S, D138Y, R190S, K417T, E484K, N501Y, D614G, H655Y, K986P, V987P, T1027Y, and V1176F. In some embodiments, the spike protein comprises at least one, at least two, at least three, at least four, at least five, at least six, at least seven, at least eight, at least nine, or at least 10 mutations selected from L18F, T20N, P26S, D138Y, R190S, K417T, E484K, N501Y, D614G, H655Y, K986P, V987P, T1027Y, and V1176F. In some embodiments, the spike protein comprises each of the following mutations relative to SEQ ID NO: 14: L18F, T20N, P26S, D138Y, R190S, K417T, E484K, N501Y, D614G, H655Y, K986P, V987P, T1027Y, and V1176F. Such an adenoviral vector is referred to herein as “CoroVaxG.3-P”. CoroVaxG.3-P is optimized against the Gamma variant of coronavirus (B.1.1.28.1).

In some embodiments, the spike protein comprises one or more of the following mutations relative to SEQ ID NO: 14: T19R, Δ157-158, K417N, L452R, T478K, D614G, P681R, D950N, K986P, and V987P. In some embodiments, the spike protein comprises at least one, at least two, at least three, at least four, at least five, at least six, at least seven, at least eight, at least nine, 10 mutations selected from T19R, Δ157-158, K417N, L452R, T478K, D614G, P681R, D950N, K986P, and V987P. In some embodiments, the spike protein comprises each of the following mutations relative to SEQ ID NO: 14: T19R, Δ157-158, K417N, L452R, T478K, D614G, P681R, D950N, K986P, and V987P. Such an adenoviral vector is referred to herein as “CorovaxG.3-D”. CorovaxG.3-D is optimized against the Delta AY1 variant of coronavirus (B.1.617.2.1).

In some embodiments, the spike protein comprises one or more of the following mutations relative to SEQ ID NO: 14: T19R, Δ157-158, K417N, L452R, T478K, D614G, P681R, R682G, R683S, R685S, D950N, K986P, and V987P. In some embodiments, the spike protein comprises at least one, at least two, at least three, at least four, at least five, at least six, at least seven, at least eight, at least nine, or at least 10 mutations selected T19R, Δ157-158, K417N, L452R, T478K, D614G, P681R, R682G, R683S, R685S, D950N, K986P, and V987P. In some embodiments, the spike protein comprises each of the following mutations relative to SEQ ID NO: 14: T19R, Δ157-158, K417N, L452R, T478K, D614G, P681R, R682G, R683S, R685S, D950N, K986P, and V987P. Such an adenoviral vector is referred to herein as “CorovaxG.3-D.FR”. CorovaxG.3-D.FR is optimized against the Delay AY1 variant and additionally comprises three mutations in the furin cleavage domain (e.g. R682G, R683S, and R685S).

In some embodiments, the spike protein comprises one or more of the following mutations relative to SEQ ID NO: 14: A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, R682G, R683S, R685S, N764K, D796Y, N856K Q954H, N969K, L981F, K986P, and V987P. In some embodiments, the spike protein comprises at least one, at least two, at least three, at least four, at least five, at least six, at least seven, at least eight, at least nine, or at least 10 mutations selected from A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, R682G, R683S, R685S, N764K, D796Y, N856K Q954H, N969K, L981F, K986P, and V987P. In some embodiments, the spike protein comprises each of the following mutations relative to SEQ ID NO: 14: A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, R682G, R683S, R685S, N764K, D796Y, N856K Q954H, N969K, L981F, K986P, and V987P. Such an adenoviral vector is referred to herein as “CorovaxG.3-O.FR”. CorovaxG.3-O.FR is optimized against the omicron variant of coronavirus and comprises three mutations in the furin cleavage domain (e.g. R682G, R683S, and R685S)

In some embodiments, the adenoviral vector comprises a nucleic acid sequence encoding a spike protein. In some embodiments, the adenoviral vector comprises a nucleic acid sequence encoding a spike protein, wherein the nucleic acid sequence encoding the spike protein comprises a sequence having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 5, SEQ ID NO: 6, SEQ ID NO: 7, SEQ ID NO: 8, SEQ ID NO: 9, SEQ ID NO: 10, SEQ ID NO: 15, SEQ ID NO: 17, SEQ ID NO: 19, SEQ ID NO: 21, SEQ ID NO: 23, SEQ ID NO: 25, or SEQ ID NO: 27. In some embodiments, the nucleic acid sequence encoding the spike protein comprises the nucleic acid sequence of SEQ ID NO: 5, SEQ ID NO: 6, SEQ ID NO: 7, SEQ ID NO: 8, SEQ ID NO: 9, SEQ ID NO: 10, SEQ ID NO: 15, SEQ ID NO: 17, SEQ ID NO: 19, SEQ ID NO: 21, SEQ ID NO: 23, SEQ ID NO: 25, or SEQ ID NO: 27. In some embodiments, the nucleic acid sequence encodes a spike protein comprising the amino acid sequence of SEQ ID NO: 16, SEQ ID NO: 18, SEQ ID NO: 20, SEQ ID NO: 22, SEQ ID NO: 24, SEQ ID NO: 26, or SEQ ID NO: 28.

In some embodiments, a nucleic acid sequence encoding one or more viral antigens (e.g. emerging virus antigens) incorporated into the disclosed adenoviral vector comprises codons expressed more frequently (and desirably, most frequently) in humans than in the source emerging virus (e.g., a coronavirus). While the genetic code is generally universal across species, the choice among synonymous codons is often species-dependent. One of ordinary skill in the art would appreciate that, to achieve maximum protection against virus infection, high levels of virus antigens must be expressed in a mammalian, preferably a human, host. In this respect, the nucleic acid sequence preferably encodes the native amino acid sequence of a viral antigen, but comprises codons that are expressed more frequently in mammals (e.g., humans) than in the virus. Changing native virus codons to the most frequently used in mammals will increase expression of the viral antigen in a mammal (e.g., a human). Such modified nucleic acid sequences are commonly described in the art as “humanized,” as “codon-optimized,” or as utilizing “mammalian-preferred” or “human-preferred” codons.

In the context of the invention, a nucleic acid sequence encoding a viral antigen is said to be “codon-optimized” if at least about 60% (e.g., at least about 70%, at least about 80%, or at least about 90%) of the wild-type codons in the nucleic acid sequence are encoded by mammalian-preferred codons. That is, a viral nucleic acid sequence (e.g. an emerging virus nucleic acid sequence) is codon-optimized if at least about 60% of the codons in the nucleic acid sequence are mammalian-preferred codons.

In some embodiments, the adenoviral vector comprises a nucleic acid sequence encoding single viral antigen. For example, in some embodiments the adenoviral vector comprises a nucleic acid sequence encoding a single viral antigen. In some embodiments, the single viral antigen is a coronavirus spike protein. In some embodiments, the adenoviral vector comprises multiple nucleic acid sequences encoding different viral antigens. For example, the adenoviral vector may comprise a first nucleic acid sequence encoding a coronavirus spike protein and a second nucleic acid sequence encoding a second viral antigen, including antigens derived from other emerging viruses described above (e.g. Zika virus, influenza virus, Ebola virus, Dengue virus, West Nile Virus, Lassa virus, Nipah virus, Rift Valley fever virus (RVFV), or Chikungunya virus). Accordingly, in some aspects the disclosed adenoviral vectors are able to induce an immune response against multiple viruses in a subject. In some embodiments, a first nucleic acid sequence encoding a first viral antigen and a second nucleic acid sequence encoding a second viral antigen are operatively linked to the same promoter. In some embodiments, a first nucleic acid sequence encoding a first viral antigen and a second nucleic acid sequence encoding a second viral antigen are operatively linked to different promoters. When two or more nucleic acid sequences encoding two different viral antigens are operatively linked to a single promoter, the nucleic acid sequences may be separated, such as by an internal ribosomal entry site (IRES) or a 2A peptide (or 2A peptide-like) sequence.

### Immunomodulators

In some embodiments, the adenoviral vector further comprises a nucleic acid sequence encoding an immune modulator. The terms “immune modulator,” “immune modulator protein,” and “immunomodulator,” are used interchangeably herein and refer to a substance or protein that affects normal immune function of an organism. In some embodiments, an immune modulator stimulates immune functions of an organism, such as by activating, boosting, or restoring immune responses. In other embodiments, an immune modulator may exert a negative effect on immune function, such as by attenuating an existing immune response or preventing the stimulation of an immune response. Immune modulators may be naturally occurring substances (e.g., proteins) or may be synthetically generated compounds. Examples of naturally occurring immune modulators include, but are not limited to, cytokines, chemokines, and interleukins. Cytokines are small proteins (˜25 kDa) that are released by a variety of cell types, typically in response to an activating stimulus, and induce responses through binding to specific receptors. Examples of cytokines include, but are not limited to, interferons (i.e., IFN-α, IFN-β, IFN-γ), leukemia inhibitory factor (LIF), oncostatin M (OSM), granulocyte-macrophage colony stimulating factor (GM-CSF), granulocyte colony stimulating factor (G-CSF), tumor necrosis factors (e.g., TNF-α), transforming growth factor (TGF)-β family members (e.g., TGF-β1 and TGF-β2). Chemokines are a class of cytokines that have chemoattractant properties, inducing cells with the appropriate receptors to migrate toward the source of the chemokine. Chemokines fall mainly into two groups: CC chemokines comprising two adjacent cysteines near the amino terminus, or CXC chemokines, in which two equivalent cysteine residues are separated by another amino acid. CC chemokines include, but are not limited to, chemokine ligands (CCL) 1 to 28, and CXC chemokines include, but are not limited to, CXC ligands (CXCL) 1 to 17. Interleukins are a structurally diverse group of cytokines which are secreted by macrophages in response to pathogens and include, for example, interleukin-1 (IL-1), IL-2, IL-6, IL-12, and IL-8. Other cytokines, chemokines, and interleukins are known in the art and described in e.g., Cameron M. J., and Kelvin D. J., Cytokines, Chemokines and Their Receptors. In: Madame Curie Bioscience Database, Austin (TX): Landes Bioscience; 2000-2013. Available from: ncbi.nlm.nih.gov/books/NBK6294/. In other embodiments, an immune modulator may be synthetically or recombinantly generated. For example, an immune modulator may be a fusion protein, a chimeric protein, or any modified version of a naturally-occurring immune modulator.

In one embodiment, the adenovirus or adenoviral vector comprises a non-native nucleic acid sequence encoding the cytokine CD40 ligand (CD40L). CD40L, also known as CD154, is a member of the TNF protein superfamily that is primarily expressed on activated T-cells. CD40L-CD40 interaction is crucial for the in vivo priming of Th1 T cells via the stimulation of IL-12 secretion by APC (Grewal, I. S. and Flavell, R. A., Annual Review of Immunology, 16: 111-135 (1998)). In some embodiments, the nucleic acid sequence encoding CD40L may be mutated. For example, the non-native nucleic acid sequence may encode a CD40L that is resistant to metalloproteinase cleavage such that CD40L expression is retained at the cell membrane (as described in, e.g., Elmetwali et al., Molecular Cancer, 9: 52 (2010)). Nucleic acid sequences encoding CD40L are publicly available and may be used in the disclosed adenovirus or adenoviral vector (see, e.g., Seyama et al., Hum Genet., 97(2): 180-5 (1996); and NCBI Reference Sequence NG_007280.1).

In embodiments where the adenoviral vector comprises two or more non-native nucleic acid sequences (e.g., a first nucleic acid sequence encoding an emerging virus antigen and a second nucleic acid sequence encoding CD40L), the two or more non-native nucleic acid sequences may be operatively linked to the same promoter (e.g., to form a “bicistronic,” “multicistronic,” “or polycistronic” sequence), the two or more non-native nucleic acid sequences may be operatively linked to separate identical promoters, or the two or more non-native nucleic acid sequences may be operatively linked to separate and different promoters. When two or more nucleic acid sequences are operatively linked to a single promoter, the nucleic acid sequences desirably are separated by an internal ribosomal entry site (IRES) or a 2A peptide (or 2A peptide-like) sequence. IRESs allow for uncoupling of translation of each coding sequence thereby avoiding the generation of inactive proteins and incorrect subcellular targeting. Promoter interference or suppression also are alleviated through the use of IRESs (see, e.g., Vagner et al., EMBO Rep., 2: 893-898 (2001)). Thus, in some embodiments, the adenoviral vector may further comprise a nucleic acid sequence encoding CD40L operatively linked to an internal ribosome entry site (IRES). 2A self-cleaving peptides were first identified in Picornaviruses as an oligopeptide (usually 19-22 amino acids) located between two proteins in some members of the picornavirus family. 2A peptides have since been identified in other viruses. Advantages of using 2A peptides for multicistronic gene expression include, for example, their small size and their ability for efficient coexpression of genes that are placed between them. Indeed, genes placed downstream of different 2A peptide sequences can induce higher levels of expression as compared to IRESs (see, e.g., Szymczak, A. L. & Vignali, D. A., Expert Opin Biol Ther., 5: 627-638 (2005)).

### Capsid Modification

In certain embodiments, the adenoviral vector comprises at least one modified capsid protein. The adenovirus capsid mediates the key interactions of the early stages of the infection of a cell by the virus and is required for packaging adenovirus genomes at the end of the adenovirus life cycle. The capsid comprises 252 capsomeres, which includes 240 hexons, 12 penton base proteins, and 12 fibers (Ginsberg et al., Virology, 28: 782-83 (1966)). In one embodiment, one or more capsid proteins (also referred to herein as “coat” proteins) of the adenovirus or adenoviral vector can be manipulated to alter the binding specificity or recognition of the virus or vector for a receptor on a potential host cell. It is well known in the art that almost immediately after intravenous administration, adenovirus vectors are predominantly sequestered by the liver, with clearance of Ad5 from the bloodstream and accumulation in the liver occurring within minutes of administration (Alemany et al., J. Gen. Virol., 81: 2605-2609 (2000)). Liver sequestration of adenovirus is primarily due to the abundance of the native coxsackie and adenovirus receptor (CAR) on hepatocytes. Thus, the manipulation of capsid proteins may broaden the range of cells infected by the adenovirus or adenoviral vector or enable targeting of the adenoviral vector to a specific cell type. For example, one or more capsid proteins may be manipulated so as to target the adenovirus or adenoviral vector protein to tumor cells or tumor-associated cells. Such manipulations can include deletions of the fiber, hexon, and/or penton proteins (in whole or in part), insertions of various native or non-native ligands into portions of the capsid proteins, and the like.

In some embodiments, the adenovirus or adenoviral vector comprises a modified fiber protein. The adenovirus fiber protein is a homotrimer of the adenoviral polypeptide IV that has three domains: the tail, shaft, and knob. (Devaux et al., J. Molec. Biol., 215: 567-88 (1990), Yeh et al., Virus Res., 33: 179-98 (1991)). The fiber protein mediates primary viral binding to receptors on the cell surface via the knob and the shaft domains (Henry et al., J. Virol., 68(8): 5239-46 (1994)). The amino acid sequences for trimerization are located in the knob, which appears necessary for the amino terminus of the fiber (the tail) to properly associate with the penton base (Novelli et al., Virology, 185: 365-76 (1991)). In addition to recognizing cell receptors and binding the penton base, the fiber contributes to serotype identity. Fiber proteins from different adenoviral serotypes differ considerably (see, e.g., Green et al., EMBO J., 2: 1357-65 (1983), Chroboczek et al., Virology, 186: 280-85 (1992), and Signas et al., J. Virol., 53: 672-78 (1985)). Thus, the fiber protein has multiple functions key to the life cycle of adenovirus.

The fiber protein is “modified” in that it comprises a non-native amino acid sequence in place of a portion of the wild-type fiber amino acid sequence. For example, in some embodiments the fiber protein is “modified” in that it comprises one or more fiber protein domains from a first serotype adenovirus and a fiber knob domain from a second serotype adenovirus. For example, in some embodiments the modified fiber protein comprises a tail domain and/or a shaft domain from a first serotype adenovirus and a fiber knob domain from a second serotype adenovirus. The first serotype adenovirus and the second serotype adenovirus are not the same. In some embodiments, the first serotype adenovirus and the second serotype adenovirus are from different adenovirus subgroups. For example, the first serotype adenovirus may be a subgroup C adenovirus and the second serotype adenovirus may be a subtype B adenovirus. In some embodiments, the first serotype adenovirus and the second serotype adenovirus are different serotypes within the same adenovirus subgroup. In some embodiments, the first serotype adenovirus is a serotype 5 adenovirus. For example, in some embodiments the modified fiber protein comprises a tail domain and/or a shaft domain from a serotype 5 adenovirus. In some embodiments, the second serotype adenovirus is a serotype 3 adenovirus. For example, in some embodiments the modified fiber protein comprises a fiber knob domain from a serotype 3 adenovirus. In some embodiments, the first serotype adenovirus is a serotype 5 adenovirus and the second serotype adenovirus is a serotype 3 adenovirus. Accordingly, in some embodiments the modified fiber protein comprises a tail domain and/or a shaft domain from a serotype 5 adenovirus and a fiber knob domain from a serotype 3 adenovirus.

Serotype 5 adenovirus entry into cells is mediated by an initial binding step to its primary receptor, the coxsackie and adenovirus receptor (CAR). CAR exhibits reduced expression on the surface of many neoplastic cells, however. In contrast, most cells express high levels of the receptors for serotype 3 adenovirus, CD46 and Desmoglein-2 (DSG-2). Thus, modification of the serotype 5 adenovirus fiber protein has been shown to substantially improve infectivity for human tumor cells. In some embodiments, at least a portion of the wild-type fiber protein (e.g., the fiber tail, the fiber shaft, the fiber knob, or the entire fiber protein) of an adenoviral vector comprising at least a portion of a serotype 5 adenovirus genome is removed and replaced with a corresponding portion of a fiber protein from an adenovirus of a different serotype (such as those described herein). In one embodiment, the knob domain of the fiber protein of a serotype 5 adenovirus or adenoviral vector is removed and replaced with a corresponding fiber knob domain of a different adenovirus serotype. For example, the fiber protein of the serotype 5 adenoviral vector may comprise a knob domain from a serotype 3 adenovirus. Other regions of the serotype 5 adenovirus fiber protein (i.e., the shaft and/or tail domains) may be removed and replaced with corresponding regions from other adenovirus serotypes (e.g., serotype 3). In one embodiment, the entire wild-type fiber protein of the serotype 5 adenovirus or adenoviral vector is replaced with the entire fiber protein of a serotype 3 adenovirus. Exchanging regions of serotype 5 adenovirus fiber protein for corresponding serotype 3 regions is described in, e.g., U.S. Pat. Nos. 5,846,782 and 7,297,542. Amino acid sequences of adenovirus serotype 3 fiber protein have been characterized and are publicly available (see, e.g., Signas, et al., J Virol., 53(2): 672-678 (1985); and UniProtKB/Swiss-Prot Accession No. P04501).

In another embodiment, at least a portion of the wild-type fiber protein of the adenoviral vector is removed and replaced with a non-adenovirus (i.e., heterologous) amino acid sequence. For example, the knob domain of the fiber protein of the serotype 5 adenovirus or adenoviral vector may be removed and replaced with an amino acid sequence or motif that has been synthetically or recombinantly generated. A few heterologous peptides have been introduced into the fiber knob domain to re-target the adenovirus, including oligo lysine, FLAG, RGD-4C RGS(His)6, and HA epitope. Due to the rather complex structure of the fiber knob domain, however, any heterologous peptide or amino acid sequence introduced into the fiber knob should not destabilize the fiber, which would render it incapable of trimerization and, hence, non-functional. Thus, any suitable heterologous amino acid sequence may be incorporated into the fiber knob domain, so long as the fiber protein is able to trimerize. In one embodiment, the fiber knob of the adenovirus or adenoviral vector described herein may be removed and replaced with a trimerization motif and a receptor-binding ligand.

The receptor-binding ligand (e.g. the ligand) may be any suitable molecule or peptide that specifically recognizes a cell surface protein that is not a native adenovirus receptor. Examples of suitable ligands include, but are not limited to, physiological ligands, anti-receptor antibodies, and cell-specific peptides. In one embodiment, the ligand is an antibody, antibody fragment, or a derivative of an antibody. In one embodiment, the ligand is an antibody fragment. Examples of antibody fragments include, but are not limited to, (i) a Fab fragment, which is a monovalent fragment consisting of the VL, VH, CL, and CH domains, (ii) a F(ab′)2 fragment, which is a bivalent fragment comprising two Fab fragments linked by a disulfide bridge at the hinge region, (iii) a Fv fragment consisting of the VL and VH domains of a single arm of an antibody, (iv) a Fab′ fragment, which results from breaking the disulfide bridge of an F(ab′)2 fragment using mild reducing conditions, (v) a disulfide-stabilized Fv fragment (dsFv), and (vi) a single domain antibody (sdAb), which is an antibody single variable region domain (VH or VL) polypeptide that specifically binds an antigen.

In other embodiments, the fiber protein comprises a non-native amino acid sequence that binds αvβ3, αvβ5, or αvβ6 integrins. Adenoviruses displaying ligands specific for αvβ3 integrin, such as an RGD motif, infect cells with a greater number of αvβ3 integrin moieties on the cell surface compared to cells that do not express the integrin to such a degree, thereby targeting the vectors to specific cells of interest. For example, the adenovirus or adenoviral vector may comprise a chimeric fiber protein comprising a non-native amino acid sequence comprising an RGD motif including, but not limited to, CRGDC (SEQ ID NO: 11), CXCRGDCXC (SEQ ID NO: 12), wherein X represents any amino acid, and CDCRGDCFC (SEQ ID NO: 13). The RGD motif can be inserted into the adenoviral fiber knob region, preferably in an exposed loop of the adenoviral knob, such as the HI loop.

Other regions of the adenovirus fiber protein (e.g. the serotype 5 adenovirus fiber protein) (i.e., the shaft and/or tail domains) may be removed and replaced with corresponding regions from other adenovirus serotypes or non-adenovirus peptides. Any suitable amino acid residue(s) of the wild-type fiber protein of the disclosed adenovirus or adenoviral vector can be modified or removed, so long as viral capsid assembly is not impeded. Similarly, amino acids can be added to the fiber protein as long as the fiber protein retains the ability to trimerize. Such modified fiber proteins also are referred to as “chimeric” fiber proteins, as they comprise amino acid sequences obtained or derived from two different adenovirus serotypes.

In some embodiments, the adenovirus or adenoviral vector comprises a modified hexon protein. The adenovirus hexon protein is the largest and most abundant protein in the adenovirus capsid. The hexon protein is essential for virus capsid assembly, determination of the icosahedral symmetry of the capsid (which in turn defines the limits on capsid volume and DNA packaging size), and integrity of the capsid. In addition, the hexon protein is a primary target for modification to reduce neutralization of adenoviral vectors (see, e.g., Gall et al., J. Virol., 72: 10260-264 (1998), and Rux et al., J. Virol., 77(17): 9553-9566 (2003)). The major structural features of the hexon protein are shared by adenoviruses across serotypes, but the hexon protein differs in size and immunological properties between serotypes (Jornvall et al., J. Biol. Chem., 256(12): 6181-6186 (1981)). A comparison of 15 adenovirus hexon proteins revealed that the predominant antigenic and serotype-specific regions of the hexon protein appear to be in loops 1 and 2 (i.e., LI or l1, and LII or l2, respectively), within which are seven to nine discrete hypervariable regions (HVR1 to HVR 7 or HVR9) varying in length and sequence between adenoviral serotypes (Crawford-Miksza et al., J. Virol., 70(3): 1836-1844 (1996), and Bruder et al., PLoS ONE, 7(4): e33920 (2012)).

The hexon protein is “modified” in that it comprises a non-native amino acid sequence in addition to or in place of a wild-type hexon amino acid sequence of the serotype 5 adenovirus or adenoviral vector. The Ad5 hexon protein mediates liver sequestration of the virus (Waddington et al., Cell, 132: 397-409 (2008); Vigant et al., Mol. Ther., 16: 1474-1480 (2008); and Kalyuzhniy et al., Proc. Natl. Acad. Sci. USA, 105: 5483-5488 (2008)), and modification of the hexon protein, specifically within the hypervariable 5 (HVR5) and 7 (HVR7) regions, has been shown to mitigate the endogenous liver sequestration of serotype 5 adenovirus particles (see, e.g., Alba et al., Blood, 114: 965-971 (2009); Shashkova et al., Mol. Ther., 17: 2121-2130 (2009); and Short et el., Mol Cancer Ther., 9(9): 2536-2544 (2010)). In some embodiments, at least a portion of the wild-type hexon protein (e.g., the entire hexon protein) of the disclosed serotype 5 adenoviral vector may be removed and replaced with a corresponding portion of a hexon protein from an adenovirus of a different serotype (such as those described herein). For example, the hexon protein of the serotype 5 adenoviral vector may comprise one or more hypervariable regions (HVRs) from an adenovirus of a different serotype (e.g., a serotype 3 adenovirus). Any suitable amino acid residue(s) of the wild-type hexon protein of the disclosed serotype 5 adenovirus or adenoviral vector can be modified or removed, so long as viral capsid assembly is not impeded. Similarly, amino acids can be added to the hexon protein as long as the structural integrity of the capsid is maintained. Such modified hexon proteins also are referred to as “chimeric” hexon proteins, as they comprise amino acid sequences obtained or derived from two different adenovirus serotypes.

Methods for generating modified (e.g., chimeric) adenovirus hexon and fiber proteins known in the art can be used in the context of the present disclosure. Such methods are described in, for example, U.S. Pat. Nos. 5,543,328; 5,559,099; 5,712,136; 5,731,190; 5,756,086; 5,770,442; 5,846,782; 5,871,727; 5,885,808; 5,922,315; 5,962,311; 5,965,541; 6,057,155; 6,127,525; 6,153,435; 6,329,190; 6,455,314; 6,465,253; 6,576,456; 6,649,407; 6,740,525; and 6,815,200.

While a variety of adenoviral vectors comprising various combinations of promoters, non-native nucleic acid sequences, adenoviral genome deletions, and chimeric fiber proteins are encompassed by the present disclosure, particular embodiments include serotype 5 adenoviral vectors comprising the following: (1) (a) a hybrid promoter comprising a CMV immediate early enhancer, a chicken β-actin promoter, and a chimeric intron comprising a splice donor site from the β-actin gene and a MVM splice acceptor site, (b) a deletion of all or part of the E1 region of the adenoviral genome, (c) a nucleic acid sequence encoding a coronavirus spike antigen operatively linked to the hybrid promoter, and (d) a fiber protein comprising a serotype 3 adenovirus fiber knob domain; (2) (a) a hybrid promoter comprising a CMV immediate early enhancer, a chicken β-actin promoter, and a chimeric intron comprising a splice donor site from the β-actin gene and a MVM splice acceptor site, (b) a deletion of all or part of the E1 region of the adenoviral genome, (c) a nucleic acid sequence encoding a coronavirus spike antigen operatively linked to the hybrid promoter (d) a nucleic acid sequence encoding CD40L operatively linked to the hybrid promoter or a different promoter, and (e) a fiber protein comprising a serotype 3 adenovirus fiber knob domain; and (3) (a) a hybrid promoter comprising a CMV immediate early enhancer, a chicken β-actin promoter, and a chimeric intron comprising a splice donor site from the β-actin gene and a MVM splice acceptor site, (b) a deletion of all or part of the E1 region of the adenoviral genome, (c) a nucleic acid sequence encoding a coronavirus spike antigen operatively linked to the hybrid promoter (d) a nucleic acid sequence encoding CD40L operatively linked to the hybrid promoter or a different promoter, and (d) a serotype 5 adenovirus fiber knob domain.

### Compositions

The disclosure further provides a composition comprising the adenoviral vector described herein and a carrier therefor (e.g., a pharmaceutically acceptable carrier). The composition desirably is a physiologically acceptable (e.g., pharmaceutically acceptable) composition, which comprises a carrier, preferably a physiologically (e.g., pharmaceutically) acceptable carrier, and the adenoviral vector. Any suitable carrier can be used within the context of the disclosure, and such carriers are well known in the art. The choice of carrier will be determined, in part, by the particular use of the composition (e.g., administration to an animal) and the particular method used to administer the composition. In some embodiments, the pharmaceutical composition can be sterile.

Suitable compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the adenoviral vector is part of a composition formulated to protect the adenovirus or adenoviral vector from damage prior to administration. For example, the composition can be formulated to reduce loss of the adenoviral vector on devices used to prepare, store, or administer the adenoviral vector, such as glassware, syringes, or needles. The composition can be formulated to decrease the light sensitivity and/or temperature sensitivity of the adenovirus or adenoviral vector. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the adenovirus or adenoviral vector and facilitate its administration. Formulations for adenoviral vector-containing compositions are further described in, for example, U.S. Pat. Nos. 6,225,289, 6,514,943, 7,456,009, 7,888,096; 10,272,032 and International Patent Application Publication WO 2000/034444.

In addition, one of ordinary skill in the art will appreciate that the adenoviral vector can be present in a composition with other therapeutic or biologically-active agents. For example, factors that control inflammation, such as ibuprofen or steroids, can be part of the composition to reduce swelling and inflammation associated with in vivo administration of the adenoviral vector. Antibiotics, i.e., microbicides and fungicides, can be present to treat existing infection and/or reduce the risk of future infection, such as infection associated with virus administration. In addition, immune system stimulators or adjuvants, e.g., interleukins, lipopolysaccharide, and double-stranded RNA, may be included in the composition to enhance or modify any immune response to the immunogen.

The dose of adenovirus or adenoviral vector present in the composition will depend on a number of factors, including the intended target tissue, the extent of any side-effects, the particular route of administration, and the like. The dose ideally comprises an “effective amount” of an adenovirus or adenoviral vector, i.e., a dose of adenovirus or adenoviral vector which provokes a desired immune response in a recipient (e.g., a human). For example, the composition may comprise a therapeutically effective amount of the adenoviral vector described herein. A “therapeutically effective amount” refers to an amount effective, at dosages and for periods of time necessary, to achieve a desired therapeutic result. Alternatively, the pharmacologic and/or physiologic effect may be prophylactic, i.e., the effect completely or partially prevents a disease or symptom thereof. In this respect, the disclosed composition comprises a “prophylactically effective amount” of the adenoviral vector. A “prophylactically effective amount” refers to an amount effective, at dosages and for periods of time necessary, to achieve a desired prophylactic result (e.g., prevention of subsequent infection and/or disease onset).

Desirably, a single dose of adenoviral vector comprises at least about 1×10⁷ particles (which also is referred to as particle units (pu) or virus particles (vp)) of the adenoviral vector. The dose is at least about 1×10⁸ particles (e.g., about 1×10⁹-1×10¹⁴ particles), or at least about 1×10¹⁰ particles, (e.g., about 1×10¹⁰-1×10¹² particles) of the adenoviral vector. Alternatively, the dose comprises no more than about 1×10¹⁴ particles, preferably no more than about 1×10¹³ particles, and more preferably no more than about 1×10¹² particles. In other words, a single dose of adenoviral vector can comprise, for example, about 1×10⁷ virus particles, 2×10⁷ vp, 4×10⁷ vp, 1×10⁸ vp, 2×10⁸vp, 4×10⁸ vp, 1×10⁹ vp, 2×10⁹ vp, 4×10⁹ vp, 1×10¹⁰ vp, 2×10¹⁰ vp, 4×10¹⁰ vp, 1×10¹¹ vp, 2×10¹¹ vp, 4×10¹¹ vp, 1×10¹² vp, 2×10¹² vp, 4×10¹² vp, 1×10¹³ vp, 2×10¹³ vp, 4×10¹³ vp, or 1×10¹⁴ vp of the adenoviral vector.

### Immunization Methods

The disclosure also provides a method of inducing an immune response against one or more viruses in a subject by administering an adenoviral vector encoding one or more viral antigens or a composition comprising the adenoviral vector, as described above. In some embodiments, the disclosure proves a method of inducing an immune response against a coronavirus in a subject, which comprises administering to the subject a composition comprising a pharmaceutically acceptable carrier and a chimeric, replication incompetent adenoviral vector comprising (a) a hybrid promoter comprising an exogenous intron, (b) a nucleic acid sequence encoding a viral antigen operatively linked to the hybrid promoter, (c) a post-transcriptional regulatory element; and (d) a modified fiber protein, whereupon the nucleic acid sequence encoding the viral antigen is expressed in the subject, thereby inducing an immune response against the virus in the subject. In some embodiments, the viral antigen is a coronavirus antigen. After administration of such a composition to the subject, the coronavirus spike protein is expressed in the subject, thereby inducing an immune response against the coronavirus in the subject. The disclosure also provides the use of a composition comprising the above-described adenoviral vector in the preparation of a medicament. For example, the disclosure provides the use of a composition for immunizing a subject against a virus, the composition comprising a chimeric, replication incompetent adenoviral vector comprising (a) a hybrid promoter comprising an exogenous intron, (b) a nucleic acid sequence encoding a viral antigen operatively linked to the hybrid promoter, (c) a post-transcriptional regulatory element; and (d) a modified fiber protein. In some embodiments, the disclosure provides use of a composition for immunizing a subject against a coronavirus, wherein the composition comprises a pharmaceutically acceptable carrier and a chimeric, replication incompetent adenoviral vector comprising (a) a hybrid promoter comprising an exogenous intron, (b) a nucleic acid sequence encoding a viral antigen operatively linked to the hybrid promoter, (c) a post-transcriptional regulatory element; and (d) a modified fiber protein. The composition may be administered to an animal, such as a mammal, particularly a human, using standard administration techniques and routes. Suitable administration routes include, but are not limited to, oral, intravenous, intraperitoneal, subcutaneous, pulmonary, transdermal, intramuscular, intranasal, buccal, sublingual, or suppository administration. In some embodiments, the administration route brings the composition into direct contact with a mucosal membrane. Suitable methods involving contact with a mucosal membrane include, for example, oral capsules, tonsillar swabs, and intranasal administration (e.g. intranasal spray). In some embodiments, the composition is suitable for parenteral administration. The term “parenteral,” as used herein, includes intravenous, intramuscular, subcutaneous, rectal, vaginal, and intraperitoneal administration. In some embodiments, the composition is administered to a subject intravenously, intramuscularly, intranasally, orally, or subcutaneously.

Cytokines play a role in directing the immune response. Helper (CD4+) T cells orchestrate the immune response of mammals through production of soluble factors that act on other immune system cells, including B and other T cells. Most mature CD4+T helper cells express one of two cytokine profiles: Th1 or Th2. Th1-type CD4+ T cells secrete IL-2, IL-3, IFN-γ, GM-CSF and high levels of TNF-α. Th2 cells express IL-3, IL-4, IL-5, IL-6, IL-9, IL-10, IL-13, GM-CSF and low levels of TNF-α. Th1 type cytokines promote both cell-mediated immunity, and humoral immunity that is characterized by immunoglobulin class switching to IgG2a in mice and IgG1 in humans. Th1 responses may also be associated with delayed-type hypersensitivity and autoimmune disease. Th2 type cytokines induce primarily humoral immunity and induce class switching to IgG1 and IgE. The antibody isotypes associated with Th1 responses generally have neutralizing and opsonizing capabilities, whereas those associated with Th2 responses are associated more with allergic responses.

The immune response induced in the mammal can be a humoral immune response, a cell-mediated immune response, or a combination of humoral and cell-mediated immunity. With respect to viral infections, “humoral immunity” occurs when virus and/or virus-infected cells stimulate B lymphocytes to produce antibody that is specific for viral antigen. IgG, IgM, and IgA antibodies have all been shown to exert antiviral activity. Antibodies can neutralize virus by (1) blocking virus-host cell interactions or (2) recognizing viral antigens on virus-infected cells which can lead to antibody-dependent cytotoxic cells (ADCC) or complement-mediated lysis. IgG antibodies are responsible for most antiviral activity in serum, while IgA is the most important antibody when viruses infect mucosal surfaces. In some embodiments, administration of the adenoviral vector described herein induces a neutralizing antibody response against an emerging virus, such as a coronavirus.

The term “cell-mediated immunity” encompasses (1) the recognition and/or killing of virus and virus-infected cells by leukocytes and (2) the production of different soluble factors (cytokines) by these cells when stimulated by virus or virus-infected cells. Cytotoxic T lymphocytes, natural killer (NK) cells, and antiviral macrophages can recognize and kill virus-infected cells. Helper T cells can recognize virus-infected cells and produce a number of important cytokines. Cytokines produced by monocytes (monokines), T cells, and NK cells (lymphokines) play important roles in regulating immune functions and developing antiviral immune functions (Klimpel G R. Immune Defenses. In: Baron S, editor. Medical Microbiology. 4th edition. Galveston (TX): University of Texas Medical Branch at Galveston; 1996. Chapter 50. Available from: ncbi.nlm.nih.gov/books/NBK8423/). Ideally, the administration of the adenoviral vector induces a T cell immune response. The immune response desirably provides protection to the animal, typically a mammal such as a human, upon subsequent challenge with a coronavirus.

In some embodiments, the method induces memory T cells directed against a coronavirus. The term “memory T cell,” as used herein, can be defined as a CD8+ T cell that has responded to a cognate antigen and persists long-term. Compared to naïve cells of the same antigen-specificity, memory T cells persist in greater numbers; can populate peripheral organs; are poised to immediately proliferate, execute cytotoxic functions, and secrete effector cytokines upon antigenic re-encounter; and exist in different metabolic, transcriptional, and epigenetic states (Homann et al., Nat Med., 7: 913-9. doi: 10.1038/90950 (2001); Masopust et al., J Immunol., 172: 4875-82. doi: 10.4049/jimmunol.172.8.4875 (2004); DiSpirito J R, Shen H, Cell Res., 20: 13-23. doi: 10.1038/cr.2009.140 (2010); Veiga-Fernandes H, Rocha B., Nat Immunol. 5: 31-7. doi: 10.1038/ni1015 (2004); and Lalvani et al., J Exp Med., 186: 859-65 (1997)). As such, hosts possessing memory T cells are often better protected against solid tumors and infection with intracellular bacteria, viruses, and protozoan parasites than their naïve counterparts (Martin, M. D., Badovinac, V. P., Frontiers in Immunology, 9: 2692 (2018)). Two major subsets of memory T cells have been identified: CD62L^{lo}/CCR7^{lo} effector memory T cells (Tem) and CD62L^{hi}/CCR7^{hi} central memory T cells (Tcm). Expression of CCR7 and CD62L on Tcm cells facilitates homing to secondary lymphoid organs, while Tem cells are more cytolytic and express integrins and chemokine receptors necessary for localization to inflamed tissues (Sallusto et al., Nature, 401: 708-12. doi: 10.1038/44385 (1999)).

While it is not yet known whether pre-existing memory T cells in humans have the potential to recognize SARS-CoV-2, CD4+ and CD8+ T cells that recognized multiple regions of the N protein have been found in individuals convalescing from COVID-19 (Le Bert et al., Nature, 584: 457-462 (2020)). This same study showed that patients who recovered from SARS (the disease associated with SARS-CoV infection) possess long-lasting memory T cells that are reactive to the N protein of SARS-CoV 17 years after the outbreak of SARS in 2003; these T cells displayed robust cross-reactivity to the N protein of SARS-CoV-2. SARS-CoV-2-specific T cells also were detected in individuals with no history of SARS, COVID-19, or contact with individuals who had SARS and/or COVID-19. These results suggest that infection with coronaviruses induces multi-specific and long-lasting T cell immunity against at least the structural N protein.

In some embodiments, the method comprises a single administration of the composition to the mammal. In other embodiments, administering the composition comprising the adenoviral vector can be one component of a multistep regimen for inducing an immune response against coronavirus in a mammal. In particular, the disclosed method can represent one arm of a prime and boost immunization regimen. In some embodiments, the method comprises multiple administrations of the composition to the mammal. For example, the first administration may be referred to as the “prime,” while subsequent administrations of the composition may be referred to as “boosts” or “boosters.” More than one booster may be provided in any suitable timeframe (e.g., at least about 1 week, 2 weeks, 4 weeks, 8 weeks, 12 weeks, 16 weeks, or more following priming) to maintain immunity.

In some embodiments, a composition comprising a first adenoviral vector is administered to the subject as the “prime” and a composition comprising the same adenoviral vector is administered to the subject as one or more “boosts”. In some embodiments, the method comprises providing a composition comprising a first adenoviral vector as the “prime” and a composition comprising a second adenoviral vector as a “boost”. In some embodiments, the first adenoviral vector and the second adenoviral vector are the same. In such embodiments, the first adenoviral vector and the second adenoviral vector are different. In some embodiments, the prime is administered by injection (e.g. intramuscular, intravenous, subcutaneous) and at least one boost is administered to the subject by bringing the composition into contact with a mucosal membrane (e.g. oral capsule, tonsillar swab, intranasally). In some embodiments, at least one boost is delivered intranasally.

In some embodiments, the adenoviral vector delivered as the boost is an adenoviral vector described herein comprising a nucleic acid sequence encoding a spike protein containing one or more mutations relative to SEQ ID NO: 14, wherein the one or more mutations comprise D614G, K986P, V987P, R682G, R683S, and R685S. In some embodiments, such an adenoviral vector is delivered intranasally to the subject as a boost. In some embodiments, a composition comprising the same or a different adenoviral vector is delivered to the subject by injection (e.g. intramuscular, intravenous, subcutaneous) as a prime. For example, in some embodiments a composition comprising the adenoviral vector CoroVaxG.3-D.FR is administered to the subject as the boost, regardless of which adenoviral vector was delivered to the subject as the prime. In some embodiments, a composition comprising the adenoviral vector CoroVaxG.3-D.FR is administered to the subject intranasally as the boost.

In some embodiments, a composition comprising a first adenoviral vector is administered to the subject as a prime, and a boost (e.g. a composition comprising the first adenoviral vector or a second, different adenoviral vector) is administered to the subject about 14-35 days after the prime. In some embodiments, the boost is administered to the subject 14, 15, 16, 17, 18, 19, 20, 21, 22, 23, 24, 25, 26, 27, 28, 29, 30, 31, 32, 33, 34, or 35 days after the prime. In some embodiments, the spacing between the prime and the boost is about 28 days.

In some embodiments, the prime and at least one boost are delivered to the subject by the same route. For example, in some embodiments the prime and at least one boost are delivered intranasally to the subject. In some embodiments, at least one boost is delivered intranasally to the subject. In some embodiments, the prime is administered parenterally (e.g. by injection) and at least one boost is administered intranasally. For example, the prime may be administered by intramuscular, intravenous, or subcutaneous injection and at least one boost may be administered intranasally. Such embodiments may be particularly beneficial for inducing a robust humoral and cellular immune response in the subject.

In some embodiments, the disclosed method reduces the number of booster injections of the disclosed adenoviral vector-containing compositions required to achieve a desired immune response (e.g., a protective immune response (e.g., a memory immune response)). In some embodiments, the disclosed method results in a higher proportion of recipients achieving seroconversion and/or more consistent immune responses within a population of subjects administered the immunogenic composition. In some embodiments, the present disclosure provides compositions that are useful for selectively skewing adaptive immunity toward Th1, Th2, or cytotoxic T cell responses (e.g., allowing effective immunization by distinct routes (e.g., such as via mucosa or via injection)). In some embodiments, the present disclosure provides compositions that elicit optimal responses in subjects in which most contemporary vaccination strategies are not optimally effective (e.g., in very young and/or very old populations). Ideally, the disclosed method induces a protective immune response, that is, an immune response that prevents the subject from displaying signs or symptoms of coronavirus infection upon subsequent exposure of the subject to a coronavirus. In particular, the disclosed method prevents the subject from displaying serious symptoms of coronavirus infection upon subsequent exposure of the subject to the coronavirus. Serious symptoms of SARS-CoV-2 infection, include, but are not limited to, difficulty breathing, sudden confusion, chest discomfort, inability to wake, or bluing of the face and/or lips.

Desirably, the disclosed methods reduce or inhibit transmission of a virus (e.g. an emerging virus), such as a coronavirus. In some embodiments, the disclosed methods completely block virus transmission. The term “virus transmission,” as used herein, refers to the process by which a virus enters a host through a portal of entry, replicates or disseminates within the host, and is transmitted to a new host through a portal of exit (Louten, J., Essential Human Virology. 2016: 71-92). With respect to SARS-CoV-2, recent evidence suggests that subjects fully vaccinated with an mRNA vaccine (Pfizer-BioNTech and Moderna) are less likely to transmit SARS-CoV-2 to others (see, e.g., Levine-Tiefenbrun et al., medRxiv. 2021, medrxiv.org/content/10.1101/2021.02.06.21251283v1.full.pdfexternal icon; Jones et al., Elife. 2021; 10; McEllistrem et al., Clin Infect Dis. 2021; Petter et al., medRxiv. 2021, medrxiv.org/content/10.1101/2021.02.08.21251329v1external icon; Levine-Tiefenbrun et al., Nat Med. 2021; 27(5):790-2; Marks et al. Lancet Infect Dis. 2021; and Harris et al. khubnet. 2021; khub.net/documents/135939561/390853656/Impact+of+vaccination+on+household+transmission+of+SARS-COV-2+in+England.pdf/35bf4bb1-6ade-d3eb-a39e-9c9b25a8122aexternal icon).

The disclosed method can be performed in combination with other therapeutic methods to achieve a desired biological effect in a patient. Ideally, the disclosed method may include, or be performed in conjunction with, one or more therapeutic agents or regimens that ameliorate the symptoms and signs of a coronavirus infection. With respect to SARS-CoV-2, no therapy has been proven to be beneficial in patients with mild to moderate COVID-19 who are not at high risk for disease progression. In such mild to moderate cases, treatment typically involves providing supportive care and symptomatic management to patients. In patients with mild to moderate COVID-19 who are at high risk for disease progression, anti-SARS-CoV-2 antibody-based therapies may have the greatest potential for clinical benefit during the earliest stages of infection. For these patients, the combination of bamlanivimab plus etesevimab (AIIa) or casirivimab plus imdevimab (AIIa) have received Emergency Use Authorizations (EUAs) from the FDA. Remdesivir, an antiviral agent, is currently the only drug that is approved by the FDA for the treatment of COVID-19, and is recommended for use in hospitalized patients who require supplemental oxygen. Remdesivir, however, is not recommended for patients who require mechanical ventilation. Dexamethasone, a corticosteroid, has been found to improve survival in hospitalized patients who require supplemental oxygen, with the greatest benefit observed in patients who require mechanical ventilation. The addition of tocilizumab, a recombinant humanized anti-interleukin-6 receptor monoclonal antibody, to dexamethasone therapy was found to improve survival among patients who were exhibiting rapid respiratory decompensation due to COVID-19. Therapeutic strategies for managing COVID-19 symptoms have been outlined by the CDC (see, e.g., covid19treatmentguidelines.nih.gov/management/therapeutic-management/)

The following examples further illustrate the invention but, of course, should not be construed as in any way limiting its scope.

### EXAMPLES

The following materials and methods were used in the experiments described in the Examples.

### Reagents and Cells

HEK293T (CRL-3216), Vero cells (CCL-81), Hs 729T (HTB-153) and THP-1 (TIB-202) cells were obtained from the ATCC (Manassas, VA, USA). HEK293 cells were purchased from Microbix Biosystems Inc (Mississauga, Canada) and 911 cells were already described (Lopez, Rivera et al. 2012). HEK293T-hACE2 cells were provided by Dr. Ortega from Centro de Medicina Comparada, Santa Fe, Argentina (described in (Crawford, Eguia et al. 2020)). All the cell lines were grown in the recommended medium supplemented with 15% of fetal bovine serum (Natocor, Cordoba, Argentina), 2 mM glutamine, 100 U/ml penicillin and 100 μg/ml streptomycin and maintained in a 37° C. atmosphere containing 5% CO₂. For HEK293T and HEK293T-hACE2 cell cultures, non-essential amino acids (1× final concentration) were added. Immature dendritic cells (iDC) were generated from THP-1 monocytes as previously described (Berges, Naujokat et al. 2005). To induce differentiation, THP-1 monocytes were cultured for five days in RPMI-1640 Medium (Gibco, MD, USA); 2-mercaptoethanol (0.05 mM final concentration; Gibco, MD, USA) and fetal bovine serum (10%); adding rhIL-4 (100 ng=1500 IU/ml; Peprotech, NJ, USA) and rhGM-CSF (100 ng=1500 IU/ml; Peprotech, NJ, USA). The acquired properties of iDCs were analyzed under microscope. Medium exchange was performed every 2 days with fresh cytokine-supplemented medium.

### Promoter and Fiber Selection

Promoters Pr1 and Pr2 were synthesized by Genscript (NJ, USA) with NotI/(XhoI-StuI) flanking restriction sites. Both promoters were cloned in the NotI/StuI sites of the vector pShuttle-I-XP-Luc (Lopez, Rivera et al. 2012) to obtain pShuttle-Pr1-Luc and pShuttle-Pr2-Luc. The vectors pAd-SV40-Luc, and pS-CMV-Renilla were previously described (Lopez, Rivera et al. 2012). For the selection of the most appropriate promoter, HEK293T cells grown in 24-well plates were co-transfected with 1 μg of the different plasmids and 100 ng of pS-CMV-Renilla, using Lipofectamine 2000 (Thermo Fisher Scientific, CA, USA). Twenty-four hours later, the cells were collected and assayed for Firefly and Renilla Luciferase activities using the Dual-Luciferase Reporter Assay System (Promega, WI, USA) and measured in a Genius luminometer (TECAN, Maennedorf, Switzerland). Each experiment was performed at least three times.

hAdV5/3-Renilla and hAdV5-Renilla replication-deficient adenoviral vectors were already described (Viale, Cafferata et al. 2013). Hs 729T and THP-1 cells were transduced with hAdV5-Renilla and hAdV5/3-Renilla viruses at MOI 500. Forty-eight hours later, the cells were collected and assayed for Renilla Luciferase activity as described.

### Vaccine Design and Production

The sequence of the Spike protein gene was extracted from the official GISAID reference sequence WIV04 (Okada, Buathong et al. 2020) and modified to obtain the D614G, K986P and V987P variant named D614G-PP. A cloning cassette flanked by StuI/SalI restriction sites was synthesized by Genscript (NJ, USA) including a Kozak consensus sequence (GCCACCATG), the codon optimized Spike D614G-PP, 589 bp of the woodchuck hepatitis virus posttranscriptional regulatory element (WPRE)(Donello, Loeb et al. 1998) and 222 bp of the SV40 virus late polyadenylation signal. Codon optimization was performed with the VectorBuilder software (en.vectorbuilder.com/tool/codon-optimization.html). The synthesized 4,650 bp fragment was cloned into the pShuttle-Pr2-Luc vector digested with StuI/SalI to exchange the luciferase ORF by the designed expression cassette, downstream of Pr2. The sequence of the resulting plasmid pS-Spike(D614G)-PP was confirmed by sequencing (Macrogen, Seul, Korea). To construct the non-replicating adenoviruses, the plasmid pS-Spike(D614G)-PP was linearized with PmeI and co-transformed with E1/E3- or E1-deleted adenoviral backbone vectors in electrocompetent BJ5183 bacteria. The resulting recombinant plasmids were named pCoroVaxG.3 and pCoroVaxG.5. The identity of the plasmids was confirmed by sequencing. The recombinant DNAs were linearized with PacI and transfected into 911 cells. The viruses were propagated in HEK-293 cells in CELLSTACK® cell culture chambers (Corning, Arizona, USA), purified by double CsCl density gradient centrifugation, and stored in single-use aliquots at −80° C.

### Western Blots

To assess spike expression by western blot, 1×10⁶ cells were seeded and cultured in 6-well plates. THP-1 (human monocytes), iDC, and Hs 729T (rhabdomyosarcoma) cells were transduced with CoroVax G.5, CoroVax G.3, or Ad5.C (MOI of 1000 for THP-1 and 500 for Hs 729T). Cells were washed twice with ice-cold PBS and lysed in Laemmli sample buffer 2×. Protein extracts were separated by SDS-PAGE with a 10% gel and transferred to nitrocellulose membranes (Bio-Rad Laboratories). The membranes were probed with an anti-spike receptor binding domain (RBD) antibody (40592-T63, Sino Biological) and an anti-beta-actin antibody (A4700; Sigma). After incubation with HRP-AffiniPure Goat Anti-Rabbit IgG (Jackson ImmunoResearch), chemiluminescence was detected with ECL following the manufacturer's instructions (Amersham) and digitized by Image Quant LAS 4000 (GE-Cytiva MA, USA).

### Mice Immunization

Six- to eight-week-old male BALB/c mice (obtained from the animal facility of the Veterinary School, University of La Plata, Argentina) were immunized with 10⁹ or 10¹⁰ viral particles (vp) of Ad.C (empty vector), CoroVaxG.5, or CoroVaxG.3 in 30 μl PBS via intramuscular injection in the hind leg. Serum samples for intermediate time points were obtained by submandibular bleeds for humoral immune response analyses. Final serum samples were obtained via cardiac puncture of anesthetized mice. The collected whole blood was allowed to clot at 37° C. for 1 hour before spinning down at 500×g for 10 minutes. The clarified sera were stored at −20° C. For RBD inoculations, eight-week-old male BALB/c mice were immunized with 7.5 μg of the receptor binding domain of spike protein (RBD, kindly gifted by Dr. Gamarnik at Leloir Institute) in 75 μL Complete Freund's Adjuvant (CFA, Sigma, St. Louis, MO) via subcutaneous injection and boosted 2 weeks later with 5 μg of RBD in 100 μL Incomplete Freund's Adjuvant (IFA, Sigma). Mice were bled 14 days after the boost. Mice were maintained under specific pathogen-free conditions at the Institute Leloir animal facility and all experiments were conducted in accordance with animal use guidelines and protocols approved by the Institutional Animal Care and Use Committee (IACUC protocol 69).

### ELISA

Sera from all mice were collected at different time points after immunization and evaluated for SARS-CoV-2-S-specific IgG antibodies using ELISA. Sera collected at week 4 after vaccination were also tested for SARS-CoV-2-S-specific IgG1 and IgG2a antibodies using ELISA. Briefly, ELISA plates (BRANDPLATES®, immunoGrade, BRAND GMBH+CO KG) were coated with 100 ng of recombinant SARS-CoV-2 spike protein (S1+S2 ECD, His-tag, Sino Biological) per well overnight at 4° C. in 50 μL PBS and then blocked with PBS-T/3% BSA (blocking buffer) for one hour. Plates were subsequently incubated 1 h at room temperature with three-fold dilutions of the mouse sera in blocking buffer. Plates were washed and bound specific IgG was detected with a HRP-conjugated goat anti-mouse IgG H&L antibody (ab6789, Abcam) diluted 1:10,000 in blocking buffer. Color development was performed by addition of 50 μL TMB Single Solution (Thermo Fisher Scientific). After 8 minutes, the enzyme reaction was stopped with 50 μL of 1 M sulfuric acid per well and the absorbance was measured in Bio-Rad Model 550 microplate reader (Bio-Rad Laboratories). Sera were assayed in duplicates and antibody titer represents the last reciprocal serum dilution above blank.

### ELISA for Quantification of IgG Subclasses

For IgG1 and IgG2a ELISAs, plates were coated with SARS-CoV-2 spike protein as described above. The S-specific IgG1e3 and IgG2a monoclonal antibodies (mAbs) (Invivogen) were serially diluted from 200 ng/mL to 3.125 ng/mL in blocking buffer and incubated 1 hour at room temperature. Mouse sera were diluted 1:150 or 1:1500 in blocking buffer in order to fit the linear range of the standard curve. After the plates were washed, HRP-conjugated goat anti-mouse IgG1 and IgG2a (1:20000, ab97240 and ab97245, Abcam) were added to each well and the ELISA was performed as described above. For each IgG subclass reference, a standard curve was plotted using GraphPad Prism 8.0 generating a four-parameter logistical (4PL) fit of the OD 450 nm at each serial antibody dilution. In this way, the relative levels were comparable between IgG subclasses measured on the same antigen. To compare the vaccine-induced immune response against a typical T_{H}2 inducing vaccination, the sera from 3 mice inoculated with RBD+Freund's adjuvant was included.

### Pseudovirus Construction for In Vitro Neutralization Assays

The pseudoviral particles (PVs) containing SARS-CoV2 Spike-D614G protein were generated according to the methodology described by Nie et al (Nie, Li et al. 2020), with modifications. Essentially, a replication defective Vesicular Stomatitis Virus (VSV) PV was generated in which the backbone was provided by a pseudotyped ΔG-luciferase (G*ΔG-luciferase) rVSV (Kerafast, Boston, MA, USA), which packages the expression cassette for firefly luciferase instead of VSV-G in the VSV genome. Briefly, the full length cDNA of Spike-D614G was cloned into the eukaryotic expression vector pcDNA3.1, using the EcoRV restriction site and blunt-end ligation strategy, to generate the recombinant plasmid pcDNA-3.1-Spike-D614G. HEK-293T cells growing in Optimem media (Gibco, MD, USA) with 2% of FBS were transduced with G*ΔG-VSV at a multiplicity of infection (MOI) of four. Twenty-minutes later, the cells were transfected with 30 μg of pcDNA-3.1-Spike-D614G, using Lipofectamine 3000 (Thermo Fisher Scientific, CA, USA) and incubated for six hours at 37° C., 5% CO₂. The cells were then washed four times with PBS in order to remove all the residual G*ΔG-VSV, and cultured in complete media at 37° C., 5% CO₂. After 48 hours the supernatant containing the PVs was collected, filtered (0.45-μm pore size, Millipore), and stored in single-use aliquots at −80° C. The 50% tissue culture infectious dose (TCID₅₀) of SARS-CoV-2 PV was determined in sextuplicates and calculated using the Reed-Muench method as previously described (Nie, Li et al. 2020).

### Neutralization of Authentic SARS-CoV-2 Virus

Neutralizing antibody (nAb) titers against SARS-CoV2 were defined according to the following protocol. Briefly, 50 μl of serum serially diluted two-fold from 1:5 to 1:320 were added in duplicate to a flat bottom tissue culture microtiter plate (COSTAR® 96 well plates), mixed with an equal volume of 110 PFU of a SARS-CoV-2 Wuhan Strain isolate (EPI_ISL_413016—H. Albert Einstein, Brazil, March 2020). All dilutions were made in MEM with the addition of 2.5% Fetal Bovine Serum (ThermoFisher Scientific) and the plates were incubated at 37° C. in 5% CO₂. After a 1 hour incubation the virus-sera mixtures were added to each well of a flat bottom tissue culture microtiter plate (Greiner CELLSTAR® 96 well plates) containing 5×10⁴ VERO CCL81 cells per well (90% of confluency). After 72 hours of incubation, virus neutralization (VNT) was evaluated by optical microscopy of the cell culture. Neutralizing titer was the last dilution in which a cytopathic effect (CPE) was not observed. A positive titer was equal or greater than 1:10. Sera from naïve mice were included as a negative control.

### IFN-γ Elispot

Spleens were removed from vaccinated or control BALB/c mice at 14 and 140 days post immunization and splenocytes were isolated by disaggregation through a metallic mesh. After RBC lysis (Biolegend), resuspension, and counting, the cells were ready for analysis. The IFN-γ-secreting cells were assessed using the ELISPOT mouse IFN-γ kit (R&D Systems) according to the manufacturer's protocol. Cells were cultured for 18 hours at 5×10⁵ cells per well with 2 μg/ml of a peptide pool consisting mainly of 15-mers (overlapping by 11 amino acids) covering the immunodominant sequence domains of the SARS-CoV-2 Spike protein (PEPTIVATOR® SARS-CoV-2 Prot_S; Miltenyi Biotec, Germany). The number of spots was determined using an automatic ELISPOT reader and image analysis software (CTL-IMMUNOSPOT® S6 Micro Analyzer, Cellular Technology Limited (CTL), Cleveland, USA).

### Flow Cytometry

Splenocytes from vaccinated or control BALB/c mice at 20 weeks post immunization were obtained as described above. The cells were incubated for 18 hours at 37° C. at 1.5×10⁶ cells per well with 2 μg/ml of SARS-CoV-2 Spike protein peptide pool (PEPTIVATOR® SARS-CoV-2 Prot_S, Miltenyi Biotec, Germany). Cells were stained with anti-CD8a (APC), anti-CD62L (FITC), and anti-CD44 (PE Cy7) surface markers (Biolegend). Cells were acquired on a FACSAria Fusion cytometer and analysis was performed using Flow Jo version 10.7.1.

### Statistical Analysis

All quantitative data are presented as the means t SEM from at least three independent samples. ANOVA, Tukey's multiple comparison test, and two-sample t tests were used to compare continuous outcomes between groups. Differences were considered significant if p<0.05. All analyses were conducted using GraphPad Prism software (version 8.2). The statistical tests used are indicated in the Brief Description of the Figures.

### Example 1

This example describes the construction of adenoviral vector constructs comprising a nucleic acid sequence encoding a SARS-CoV-2 spike protein operatively linked to a hybrid promoter.

### Promoter Selection

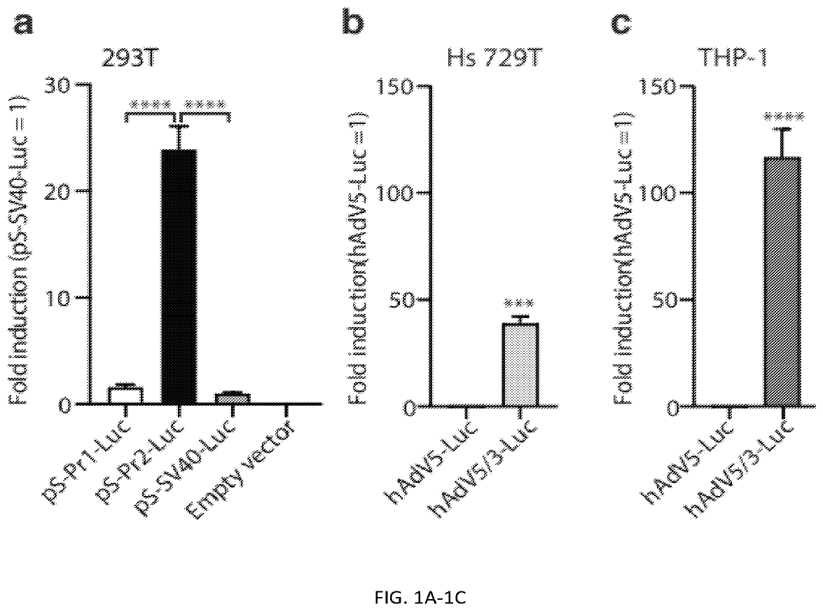

In order to induce Spike immunodominance, the first step in vaccine design was to select the most appropriate promoter to transcriptionally regulate Spike expression. Vaccines based on replication-deficient adenovirus have been generated incorporating the early intermediate CMV promoter to drive Spike transcription (REF). However, it has been extensively demonstrated that this promoter can be silenced in vivo due to methylation, histone acetylation or additional, still unclear reasons (Chen, Bailey et al. 1997, Choi, Basma et al. 2005). Although this effect was mainly observed when the CMV promoter is incorporated in viral vectors that integrate to the host genome, such as retrovirus or lentivirus (Xia, Zhang et al. 2007), silencing of the CMV promoter due to methylation has been reported in rat muscle after administration of a replication-deficient adenoviral vector (Brooks, Harkins et al. 2004). Hence, an extensive search was undertaken to select a promoter suitable for inducing long-term, high expression of Spike in an adenoviral context. Several versions of a hybrid promoter including a CMV enhancer, the chicken β-actin promoter, a chimeric intron that contains the 5′splice donor of the chicken 5′UTR β-actin and the 3′acceptor splice of the mouse Minute Virus (MMV) have been generated. This hybrid promoter has been shown to drive gene expression in the rat CNS following in vivo transduction with AAV vectors (Gray, Foti et al. 2011). Based on the available sequences of each block of this hybrid promoter, modified versions of this promoter were synthesized that differed mainly in the size of the CMV enhancer and the β-actin promoter. In addition, stop codons were incorporated in the three open reading frames after an ATG codon in the 3′ region of the promoter that could interfere with Spike ATG. In order to accelerate vaccine candidate construction, two initial versions of the hybrid promoter named Pr1 (SEQ ID NO: 1) and Pr2 (SEQ ID NO: 2) were cloned into the pShuttle(PS)-IXP-Luc vector and analyzed for their transcriptional activity. Luciferase expression driven by Pr2-Luc was around 24-fold (P<0.0001) higher than the control SV40 promoter, compared to only 1.6-fold increase over the SV40 promoter induced by Pr1-Luc (FIG. 1A). Based on these data, Pr2 was chosen for construction of the candidate adenoviral-based vaccine.

### Fiber Selection

The next step was to assess whether the adenoviral vector can be retargeted specifically to the cells that will act as antigen presenting cells, mainly dendritic and muscle cells. Previous studies have shown that exchange of AdV5 fiber knob domain with that of AdV3 enhanced the in vivo transduction of mice skeletal muscle cells(Chondronasiou, Eisden et al. 2018). Moreover, a similar hybrid AdV5.3 was able to transduce efficiently ex vivo human dendritic cells obtained from skin and lymph nodes(van de Ven, Lindenberg et al. 2009). It was also demonstrated that cells' transduction with the hybrid AdV5.3 was more efficient than AdV5 and more specific since less bystander cells were transduced with the hybrid AdV5.3(van de Ven, Lindenberg et al. 2009). Since the vaccines are to be used in a human setting, human rhabdomyosarcoma HS729T cells (as an example of muscle cells) and human THP-1 monocytes were transduced with replication-deficient adenoviruses hAdV5-Luc and the hybrid hAdV5.3-Luc. Using luciferase as a surrogate marker, a 35-fold induction and more than 100-fold induction in HS729T and THP1 cells, respectively, was observed with hAdV5.3-Luc compared to hAdV5-Luc (FIG. 1B and C).

### In Vitro Spike Expression

As mentioned above, Pr2 was selected as the promoter to drive Spike expression. In order to enhance Spike expression, the woodchuck hepatitis virus post-transcriptional regulatory element (WPRE) also was included as an mRNA stabilizer (Donello, Loeb et al. 1998). A series of viral DNA constructs were generated expressing either hAdV5 or hAdV3 fiber knob domain.

Among the six different constructs generated, two vaccine candidates (denoted “CoroVaxG.5” and “CoroVaxG.3”) were selected for further in vitro and in vivo studies. Both replication-deficient hAdV-based vaccines encoded the full-length, codon-optimized sequence of SARS-CoV2 Spike protein stabilized in its prefusion state. Spike expression in both vaccine candidates was transcriptionally regulated by Pr2 and included the WPRE mRNA stabilizer. CoroVaxG.3 was additionally modified by engineering the fiber knob domain of hAdV3 instead of the knob domain of hAdV5. A replication-deficient hAdV5 vector that carried no transgene (denoted “Ad.C”) was used as a control for further in vitro and in vivo studies.

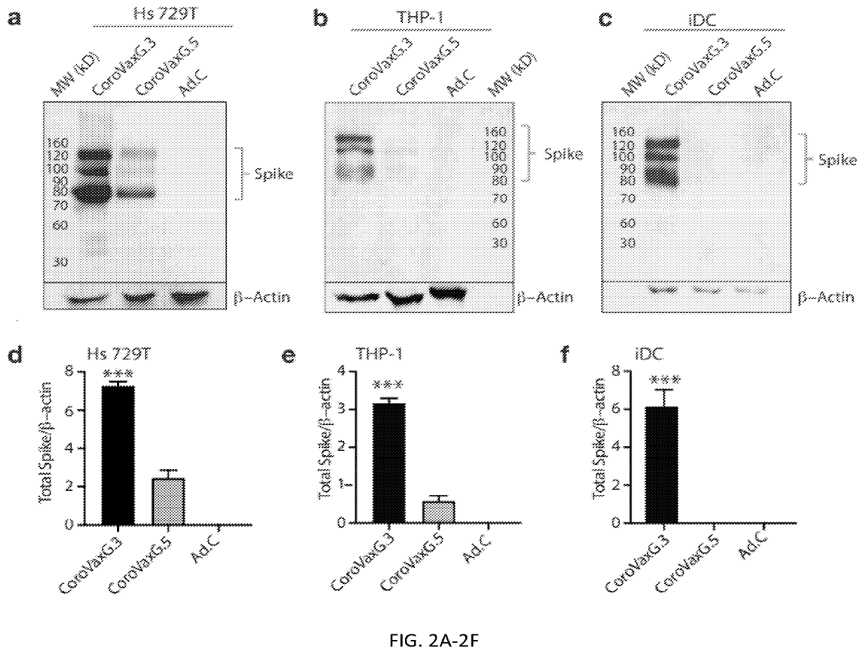

In order to establish if the fiber knob domain exchange can indeed improve targeting of muscle and dendritic cells by the viral construct, HS729T rhabdomyosarcoma cells and THP-1 monocytes were transduced with CoroVaxG.5 and CoroVaxG.3. A remarkable increase in spike expression was observed in HS729T cells transduced with CoroVaxG.3 as compared to CoroVaxG.5 (FIGS. 2A and 2D). Moreover, CoroVaxG.3 was able to induce at least six times higher Spike expression in THP-1 monocytes as compared to CoroVaxG.5 (FIGS. 2B and 2E). Interestingly, CoroVaxG.3 induced a higher increase in Spike expression in THP1 cells induced to differentiate to immature dendritic cells as compared to undifferentiated THP1 monocytes (FIGS. 2C and 2F). Spike expression was not detected in iDCs after transduction with CoroVaxG.5 even after film overexposure (FIGS. 2C and 2F).

### Example 2

This example demonstrates that the CoroVaxG vaccines induce robust and balanced antibody responses against SARS-CoV-2.

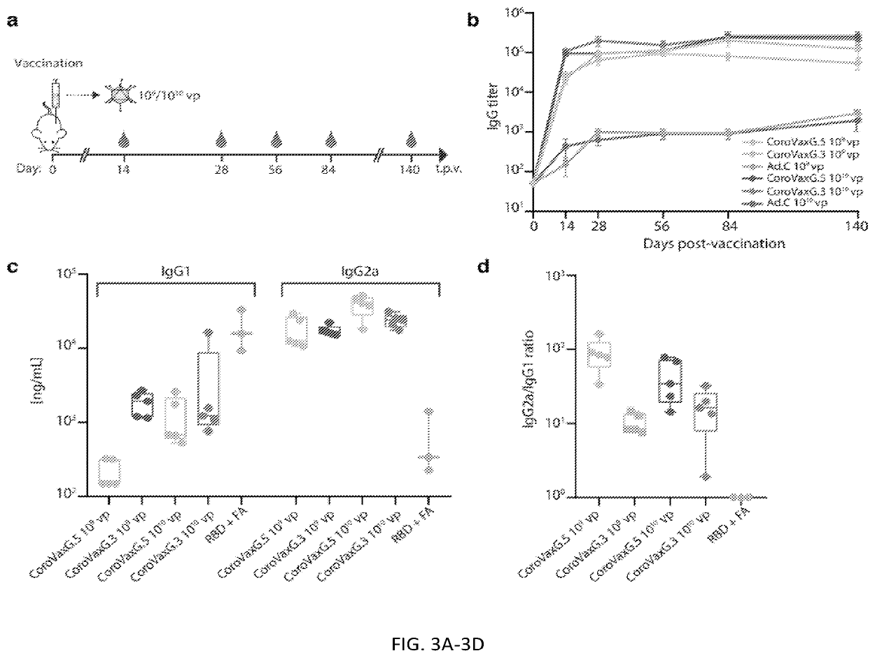

To assess the immunogenicity of CoroVaxG.5 and CoroVaxG.3, groups of 6 to 8-week-old BALB/c mice were immunized by intramuscular injection with 10⁹ or 10¹⁰ viral particles of the CoroVaxG vaccines or the control vector Ad.C. Serum samples were collected at different time points after immunization (FIG. 3A), and IgG responses against spike were evaluated by ELISA. Both CoroVaxG.5 and CoroVaxG.3 induced high levels of spike-specific IgG as early as two weeks after a single inoculation, while mice injected with Ad.C showed negligible levels of spike-specific IgG (FIG. 3B). No significant differences were observed in antibody levels between vaccine constructs or between doses, starting from 28 days post-vaccination. Moreover, these antibody titers remained stable up to 140 weeks post vaccination with a single dose (maximum time point assayed) without evidence of waning. Together, these results indicate that a single immunization with the AdV-vectored vaccine candidates via intramuscular administration is capable of generating a robust and long-lived S-specific antibody response, with no significant differences in antibody levels between doses.

To reduce the theoretical risk of vaccine-associated enhanced respiratory disease associated with a TH2-skewed response (Bournazos, Gupta et al. 2020), the concentrations of S-specific IgG1 and IgG2a subclasses induced by CoroVaxG.5 and CoroVaxG.3 were assessed as a read out of TH1 and TH2 responses. Sera from mice inoculated with the SARS-CoV2-S RBD domain in conjunction with incomplete Freund's adjuvant was used as a control, as it is known to induce a TH2-biased immune response (Shibaki and Katz 2002). IgG2a is primarily produced during a TH1-dominant immune response, whereas IgG1 is produced during any type of immune response. Therefore, an increase of the IgG2a/IgG1 ratio would reflect a skewing of the immune response towards the TH1 type. RBD+CFA induced a low IgG2a/IgG1 ratio consistent with the production of high levels of IgG1 and a clear skew towards a TH2 phenotype (FIGS. 3C and D). On the contrary, both CoroVaxG.5 and CoroVaxG.3 induced a high IgG2a/IgG1 ratio indicative of a TH1-biased response, although the IgG2a/IgG1 ratio was slightly higher for CoroVaxG.5 than for CoroVaxG.3 (FIGS. 3C and D). This difference was mainly due to a higher production of IgG1 by CoroVaxG.3, with similar levels of IgG2a in both vaccine candidates and doses (FIG. 3C).

Recent studies have shown that T cell frequencies in COVID-19 affected asymptomatic and symptomatic individuals was quite similar; however, it was of note that asymptomatic individuals showed increased production of IFN-γ and IL-2 associated with a proportional secretion of IL-10 and proinflammatory cytokines (e.g., IL-6, TNF-α, and IL-1β) (Le Bert, Clapham et al. 2021). On the contrary, symptomatic individuals secreted a disproportionate amount of inflammatory cytokines (Le Bert, Clapham et al. 2021). Thus, asymptomatic SARS-CoV-2-infected individuals, who are in essence the best controllers of symptomatic disease, raised a more efficient and balanced antiviral cellular immunity that involves a more coordinated production of proinflammatory and regulatory cytokines such as the Th2 cytokine IL-10 (Le Bert, Clapham et al. 2021). Indeed, the simultaneous production of a Th2 cytokine as IL-10 with IFN-γ served as an effective viral control without inducing a pathological process (Sun, Madan et al. 2009, Zhao, Zhao et al. 2016). Here, it is reasonable to assume that CoroVaxG.3 is triggering a more balanced IgG2a/IgG1 ratio due to the increased production of IgG1 that is maintained in the long term, while at the same time inducing IgG2a levels similar to CoroVaxG.5. Thus, CoroVaxG.3 may induce a more balanced immune response where proinflammatory cytokines associated with Th1 response are counterbalanced with production of cytokines associated with a Th2 response.

### Example 3

This example demonstrates that the adenoviral vector-based SARS-CoV-2 vaccines disclosed herein induce a strong and long-lasting T cell response.

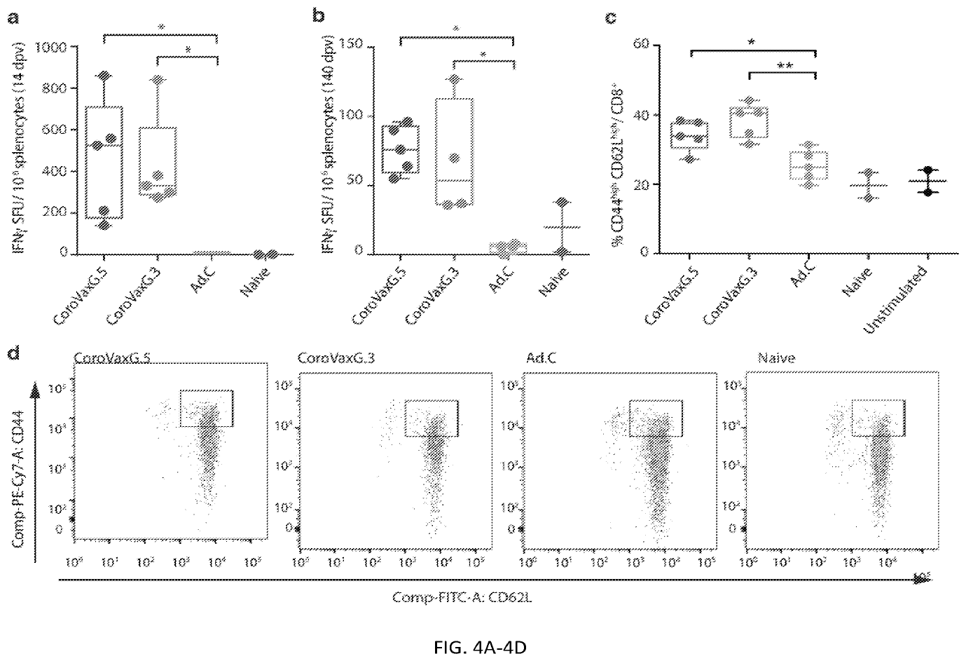

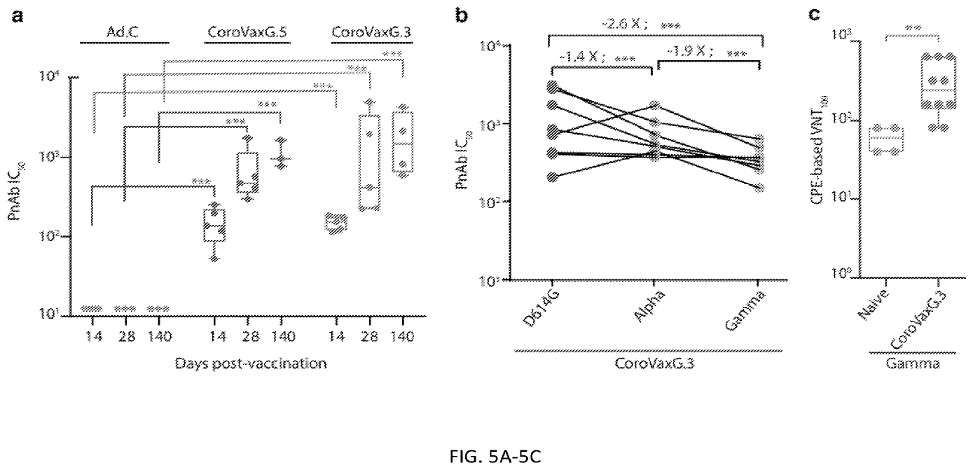

Frequently, the efficacy of prophylactic vaccines against viral infections is assessed mainly by their capacity to induce neutralizing antibodies; nevertheless, vaccines might fail to protect against viral infections in the long term. The generation and persistence of memory T cells provides life-long protection against pathogens. In particular, the induction of virus-specific CD8+ T cell responses has the potential to improve the efficacy of vaccination strategies (Remakus and Sigal 2013, Schmidt and Varga 2018). Antigen-specific naive T cells become activated upon antigen exposure, and subsequently proliferate and differentiate into effector T cells, which produce cytokines such as IFN-γ (Sallusto, Lanzavecchia et al. 2010). In order to characterize the cellular immune response induced by a single immunization with CoroVaxG.5 or CoroVaxG.3, IFN-γ production by isolated splenocytes was assessed after specific ex vivo re-stimulation with spike peptide pools. An early strong primary immune response was observed in vaccinated mice at 14 days post immunization, showing a similar range of IFN-γ secreting cells in both vaccinated groups (FIG. 4A). IFN-γ secretion was induced up to 140 days following vaccination, with no significant differences observed between CoroVaxG.5 and CoroVaxG.3 vaccines (FIG. 4B). Administration of the Ad.C control vector did not induce IFN-γ production at any of the assessed time points (FIGS. 4A and 4B).

Antigen specific long-lived memory T cells persist in vivo as a heterogeneous population in multiple sites, and can coordinate protective immune responses upon pathogen re-exposure (Remakus and Sigal 2013). The identification of distinct memory T cells is usually based on the differential cell surface expression levels of CD44 (a glycoprotein involved in cell-cell interactions, cell adhesion, and migration) and CD62L (L-selectin, a cell adhesion molecule). Effector-memory T (TEM) cells can be identified as CD44^{high} CD62L^{low} and central-memory T (TCM) cells as CD44^{high} CD62L^{high}. Secondary lymphoid organs, such as the spleen, are the main homing sites of TCM cells, whereas TEM cells are more dominantly present in non-lymphoid tissues (Sallusto, Lenig et al. 1999, Lefrancois and Masopust 2002, Wherry, Teichgraber et al. 2003, Sallusto, Lanzavecchia et al. 2010). Both subsets of memory T cells exhibit an effector function, as they produce effector cytokines in response to viruses, antigens, and other stimuli, although TCM cells exhibit a higher proliferative capacity. Therefore, the expression of CD44 and CD62L was analyzed by flow cytometry upon ex vivo stimulation with spike peptides in splenocytes obtained from mice 140 days after vaccination. High levels of CD44^{high} CD62L^{high} CD8+ cells were induced in vaccinated groups compared to control groups (FIGS. 4C and 4D). The proportion of CD44^{high} CD62L^{low} CD8+ did not change in vaccinated mice at this time point. Ex vivo stimulated splenocytes in the Ad.C group showed levels of CD8+ cells expressing CD44^{high} CD62L^{high} similar to those observed in naïve mice or unstimulated splenocytes. Collectively, the data show that mice vaccinated with CoroVaxG.5 or CoroVaxG.3 developed an effective long lasting memory T-cell response against SARS-CoV-2.

### Example 4

This example demonstrates that adenoviral vector-based vaccines described herein induce durable neutralizing antibody responses and broad coverage against SARS-CoV-2 Variants of Concern (VOCs).

To evaluate the potential resistance of new variants to neutralization elicited by CoroVaxG.3, neutralization activity against SARS-CoV-2 pseudotyped viruses containing the spike protein of the D614G reference strain (wild type) was assessed, as well as two prevalent circulating variants in South America, B.1.1.7 (alpha, first identified in the UK) and P.1 (gamma, first identified in Manaos, Brazil). Sera from 8 mice were obtained at day 28 after vaccination with 10¹⁰ vp of CoroVaxG.3 and sera from 4 naïve mice were used as a control. All naïve sera showed undetectable levels of neutralization against the tested SARS-CoV-2 variants (IC₅₀<25). The neutralizing antibody titers elicited against the wild-type strain (GMT=870.7, CI 95% 384.8-1970) showed a slight decrease (1.4 fold, P<0.01) versus B.1.117 lineage (GMT=627.1, CI 95% 402.9-976.2), and a larger but still moderate decrease (2.6 fold, P<0.01) versus P.1 lineage (GMT=334, CI 95% 232.5-480) (FIG. 5B). Interestingly, all tested sera neutralized the pseudovirus variants with IC50s of at least 150. Assessment of the same sera samples from day 28 used for the PBNA studies revealed that CoroVaxG.3 was able to elicit neutralizing antibodies against the authentic P.1 VOC (FIG. 5C).

### Example 5

It is clear that massive vaccination was the main safeguard to avoid a catastrophic development of the COVID-19 pandemic. The accumulating data of the approved SARS-CoV-2 vaccines indicate that the incidence of severe and critical disease (and deaths) in vaccinated individuals was dramatically reduced compared to the rates in the unvaccinated groups, with a positive impact in reducing the burden on healthcare systems around the world. The first major transition from the ancestral Wuhan strain in December 2019 occurred only few months later with the emerging of the D614G mutation in the stalk region of the spike protein in February 2020. The D614G variant, B.1, outcompeted the ancestral Wuhan lineage (A/B) in 3 months, clearly establishing the capacity of this virus to infect and propagate in human hosts^{1,2}. The emergence of VOCs with improved transmissibility, fast replication and partial immune escape was related mainly to mutations in the RBD domain, although relevant mutations were also observed in the NTD and at the furin cleavage site³. To date, the WHO has classified five lineages as variants of concern (VOC), which include Alpha (B.1.1.7), Beta (B.1.351), Gamma (B.1.1.28.1 or P.1), Delta (B.1.617.2) and, more recently, Omicron (B.1.1.529)⁴. These VOCs posed new challenges, which are currently being addressed by scientists and pharmaceutical companies all over the world⁵.

One of these challenges is related to the waning immunity of vaccines over time, since most VOCs exhibited reduced recognition by neutralizing antibodies. Beta was the first VOC with an immune escape profile although it never spread widely worldwide. Delta, on the other hand, rapidly outcompeted all previous lineages all over the world, and its increased transmissibility jeopardized vaccine efficacy characterized by the appearance of breakthrough infections⁶. This aspect became even more pressing with the emergence of the Omicron variant, which combined an increased transmissibility with an extensive immune escape profile. Breakthrough infection rates are under intense scrutiny and in fact, severe disease as a result of breakthrough infections has been observed at relevant rates in individuals vaccinated with two doses that decreased slightly in those that received a third dose⁷. Since current vaccines are based on the WA1 or D614G variants, there is a potential requirement for more robust vaccines, even polyvalent ones, that might induce a widespread immune response.

Described herein is the development against adenoviral vector-based vaccines against different VOC. The capacity of the vaccines described herein to induce an immunogenic response able to protect against mismatched variants was investigated. These adenoviral vaccines were engineered to express at high levels a membrane-bound Spike stabilized in a prefusion conformation. The adenoviral vectors—vaccines were retargeted to specifically transduce muscle and dendritic cells. Using these type of vaccines candidates, neutralization of pseudovirus expressing Spike corresponding to different VOC including Omicron was evaluated. Vaccines' ability to protect mice from a challenge with different VOC and their capacity in restricting VOC replication and injury in lung and brain was also evaluated.

### Methods and Results

### Construction of the Different Vaccine Candidates and In Vitro Spike Expression

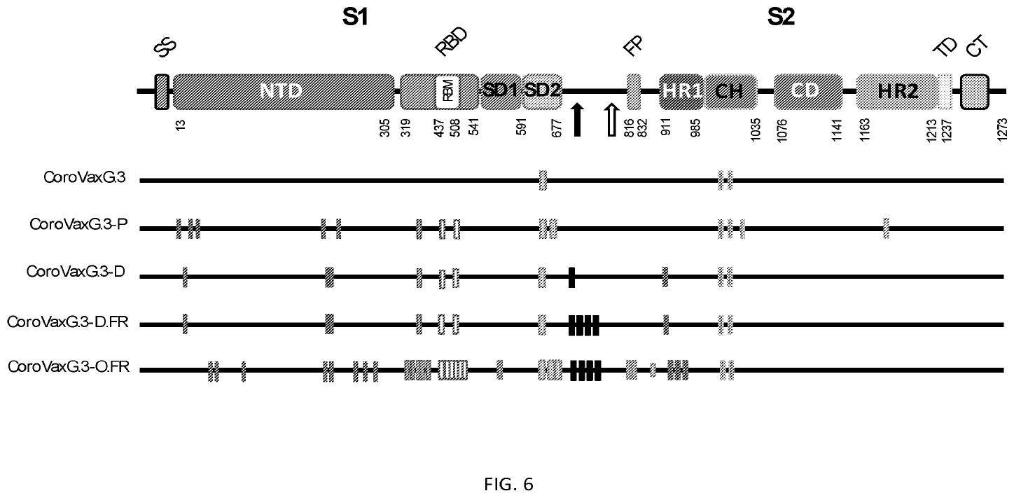

In order to produce the vaccines against the different VOC, the vaccine CoroVaxG.3, which is based on a hybrid h5/3-Adenovirus, was engineered to bear spikes from different SARS-CoV-2 variants, namely B.1 (Wuhan-1 wild type with the D614G mutation), P.1 (Gamma) and B.1.617.2.1/AY.1 (Delta plus) with the K986P/V987P mutation (PP) to stabilize them in their prefusion conformation. The two variants were selected based on their high circulation in Argentina. An additional AY.1-PP-FR (furin resistant) vaccine, with its furin cleavage site (residues 682-685) modified to “GSAS”, was constructed. Due to the emergence of the Omicron variant during the course of this study, a vaccine matched to B.1.1.529 (Omicron BA.1) Spike, named CoroVAxG.3-O.FR, which also included the PP stabilization and the furin cleavage motif mutation (FIG. 6), was also generated. All vaccines produced herein are described in Table 1 below.

TABLE 1
Mutations incorporated in Spike corresponding to each vaccine candidate

| | | | | | | Delta-PP | Delta AY1- | Omicron-BA1 |
|---|---|---|---|---|---|---|---|---|
| | | Wuhan | | | Gamma-PP | AY1 | PP-FR | PP-FR |
| | | (B.1-PP) | Alpha-PP | Beta-PP | (B.1.1.28.1) | (B.1.617.2.1) | (B.1.617.2.1) | (B.1.1.529.1) |
| Region | Subregion | CoroVaxG.3 | (B.1.1.7) | (B.1.351) | CoroVaxG.3-P | CorovaxG.3-D | CorovaxG.3-D.FR | CorovaxG.3-O.FR |
| S1 | NTD | — | Δ69-70 | L18F | L18F | T19R | T19R | A67V |
| | | | Δ144 | D80A | T20N | Δ157-158 | Δ157-158 | Δ69-70 |
| | | | | D215G | P26S | | | T95I |
| | | | | Δ242- | D138Y | | | G142D |
| | | | | 244 | R190S | | | Δ143-145 |
| | | | | R246I | | | | Δ211 |
| | | | | | | | | L212I |
| | | | | | | | | ins214EPE |
| | RBD | — | | K417N | K417T | K417N | K417N | G339D |
| | | | | | | | | S371L |
| | | | | | | | | S373P |
| | | | | | | | | S375F |
| | | | | | | | | K417N |
| | RBD- | — | N501Y | E484K | E484K | L452R | L452R | N440K |
| | RBM | | | N501Y | N501Y | T478K | T478K | G446S |
| | | | | | | | | S477N |
| | | | | | | | | T478K |
| | | | | | | | | E484A |
| | | | | | | | | Q493R |
| | | | | | | | | G496S |
| | | | | | | | | Q498R |
| | | | | | | | | N501Y |
| | | | | | | | | Y505H |
| | SD1 | — | A570D | | | | | T547K |
| | SD2 | D614G | D614G | D614G | D614G | D614G | D614G | D614G |
| | | | | | H655Y | | | H655Y |
| | | | | | | | | N679K |
| Furin | | — | P681H | | | P681R | P681R | P681H |
| Site | | | | | | | R682G | R682G |
| | | | | | | | R683S | R683S |
| | | | | | | | R685S | R685S |
| S2 | | K986P | T716Y | A701V | K986P | D950N | D950N | N764K |
| | | V987P | S982A | K986P | V987P | K986P | K986P | D796Y |
| | | | K986P | V987P | T1027Y | V987P | V987P | N856K |
| | | | V987P | | V1176F | | | Q954H |
| | | | D1118H | | | | | N969K |
| | | | | | | | | L981F |
| | | | | | | | | K986P |
| | | | | | | | | V987P |

Normal font: amino acid changes in Spike expressed by each vaccine compared to Spike expressed by B1 variant. Bold: amino acid changes to stabilize Spike in the prefusion conformation. Italics: amino acid changes that mutate the furin cleavage domain.

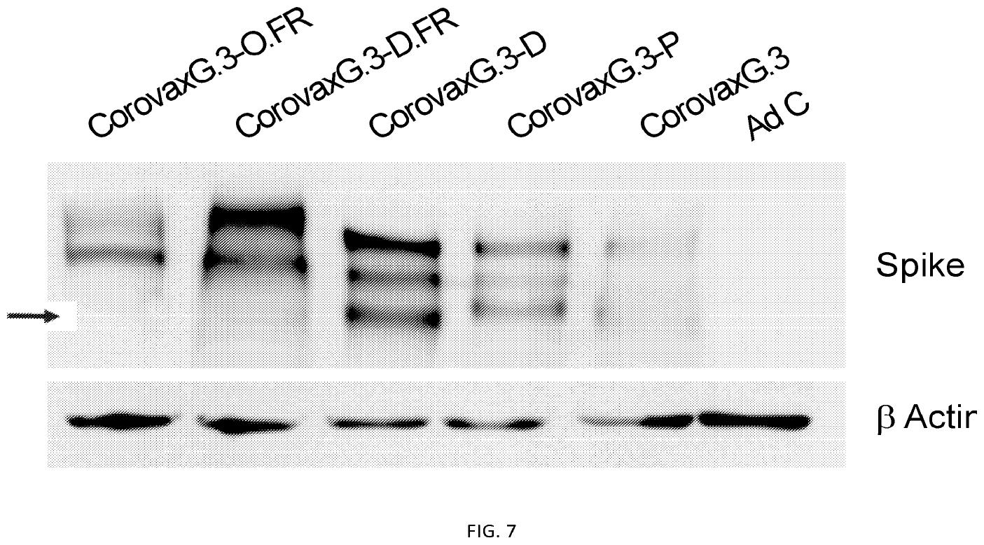

Several vaccines described in Table 1 were able to induce the expression of the respective Spike in Hs 7297 cells (FIG. 7). CoroVaxG.3-D.FR that lacks an active furin cleavage domain shows the absence of the cleavable lower band (FIG. 7).

### A Single Dose of the CoroVax Vaccine Based on Ancestral or VOC-Spikes Elicits Similar Levels of Humoral and Cellular Immunity

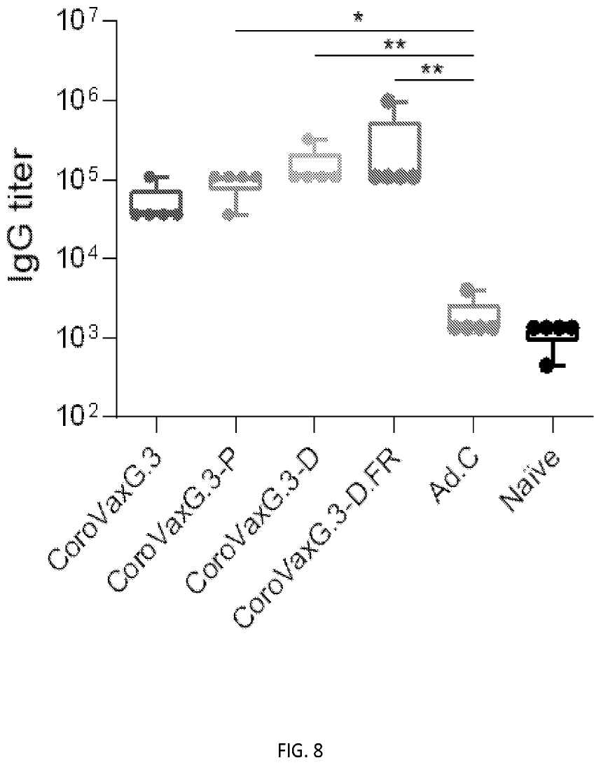

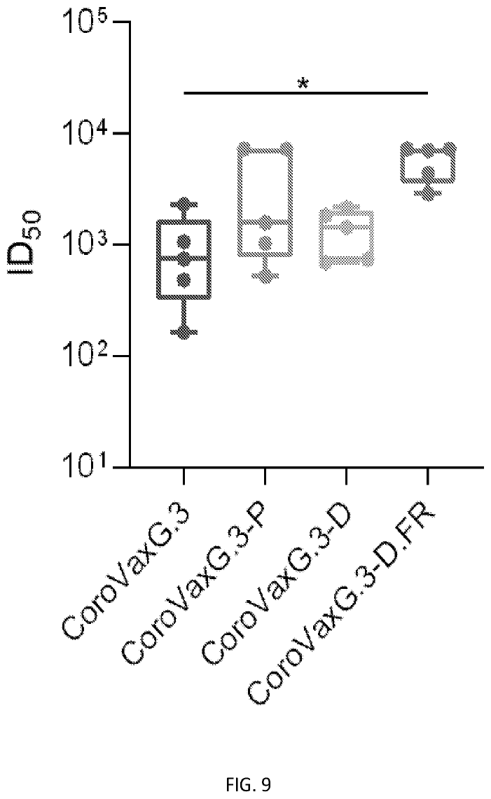

BALB/c mice were vaccinated with a single dose of Corovax.G3, Corovax.G3-P, Corovax.G3-D, or Corovax.G3-D.FR. Four weeks post-immunization, the animals were bled and the IgG responses against A.1 (Wu-1) full length Spike were evaluated by ELISA. Similar binding responses to the full-length Spike were observed between vaccinated groups (FIG. 8A) pointing to a similar immunogenicity regardless of the Spike variant included in the different vaccines. Additionally, the neutralizing activity of the sera was assessed in a well-established pseudovirus-based neutralization assay (PBNA)¹⁵. When tested against a matched virus variant from which the Spike antigen used for the vaccine was derived, all animals elicited a strong neutralizing antibody response (FIG. 9); naïve animals and mice vaccinated with an empty adenovector showed no reactivity. Although all vaccine versions showed a significant neutralization titer against their matched variant, there was a trend towards CorovaxG3-D.FR-vaccinated animals showing a higher neutralizing titer against the homologous PsV (Delta) as compared to the other vaccine candidates. This difference was subtle and only statistically significant for CorovaxG.3-D.FR versus CorovaxG.3.

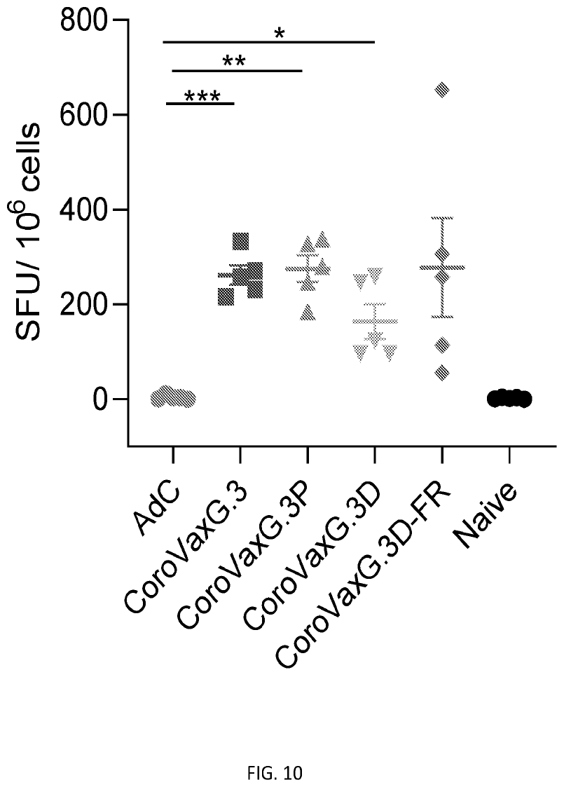

Assessment of the cellular immune response using induced by the different vaccines showed essentially no difference in vaccines' capacity to induce a cell mediated anti-SARS-CoV-2 response assessed through the use of IFN-Y production as a readout (FIG. 10).

### VOC-Matched Vaccines Induce Cross-Neutralizing Antibodies in Mice

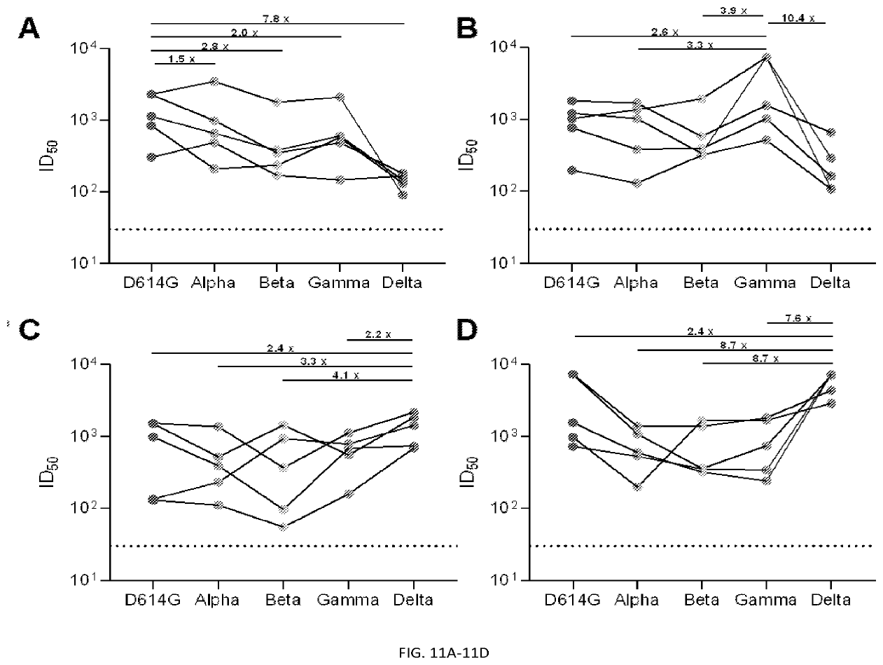

In order to establish the cross-neutralization capacity the vaccine constructs, neutralizing capability elicited by the 3 vaccines against the VOCs described to date were tested. When testing for cross-reactivity, and considering only vaccines built with the PP mutations and an active furin cleavage domain, the vaccines induced the highest neutralization titers against their homologous VOC (CorovaxG.3 for B.1, CorovaxG.3-P for Gamma and CorovaxG.3-D for Delta, respectively) (FIG. 11 and Table 2). Interestingly, the cross-neutralizing capacity of a vaccine resistant to proteolytic cleavage by furin-like proteases (in addition to the PP mutations), CorovaxG3-D.FR, displayed the highest levels of neutralization not only to its homologous VOC (Delta) but also to B.1.351 (Beta) and B.1. Of note, CorovaxG.3-D.FR neutralized the ancestral B.1 variant even better than its matched CorovaxG3 vaccine. Importantly, when considering all VOCs arisen before Omicron (Alpha to Delta), all vaccine candidates elicited a strong nAb response against all of the VOCs, either homologous or heterologous (FIG. 11).

TABLE 2
Geometric Mean Neutralization Titers of the variant-matched
vaccines against SARS-CoV-2 VOC-based pseudoviruses.

| | ID50 GMT against VOC (95% CI) | | | | |
|---|---|---|---|---|---|
| Vaccine | D614G | Alpha | Beta | Gamma | Delta |
| CoroVaxG.3 | 1085 | 744.6 | 392 | 552.4 | 138.8 |
| | (383- | (206.4- | (127.7- | (171.2- | (99.14- |
| | 3072) | 2686) | 1203) | 1783) | 194.2) |
| CoroVaxG.3-P | 808.8 | 654 | 545.3 | 2137 | 206.3 |
| | (282- | (174- | (215.6- | (488.7- | (79.9- |
| | 2320) | 2458) | 1379) | 9348) | 532.6) |
| CoroVaxG.3-D | 525.8 | 372 | 305.5 | 556.2 | 1238 |
| | (109- | (116.5- | (53.02- | (220.2- | (644.4- |
| | 2537) | 1188) | 1760) | 1405) | 2377) |
| CoroVaxG.3-D.FR | 2253 | 621.3 | 620.7 | 713.3 | 5431 |
| | (571.1- | (243.9- | (225.2- | (228.8- | (3239- |
| | 8892) | 1583) | 1711) | 2224) | 9106) |

Vaccines Cross-Protection of K18-hACE2 Transgenic Mice Against Challenge with Different SARS-CoV-2 VOC

The previous data demonstrated certain variability in the neutralizing capacity of the different CoroVaxG.3 vaccines against heterologous strains. Accordingly, it was next investigated whether this variability extends to vaccines' capacity to protect mice from challenges with the different VOC. Naïve mice were infected intranasally with different doses of the ancestral HU.1 strain, the Gamma variant, due to its massive outbreak in South America, and the Delta variant, which co-prevailed in Argentina with Gamma until the appearance of the Omicron variant. Aged K18-hACE2 mice were followed for weight loss and severe disease conditions following strict animal welfare regulations. From these studies, inoculum of Gamma and Delta strains were selected.

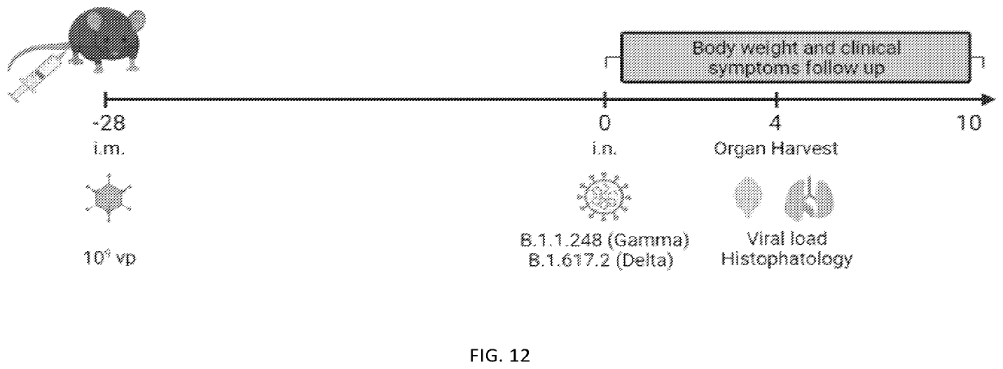

In subsequent studies, 6 to 8 months old K18-hACE2 C57BL/6J mice were immunized by administering 10⁹ particles of each of the CoroVaxG.3-based vaccines and challenged 28 days later intranasally, with the different SARS-CoV-2 variants at the selected dose (FIG. 12). A group of mice were sacrificed at day 4 to assess viral load in lungs and brains as readout of local infection and virus dissemination, respectively.

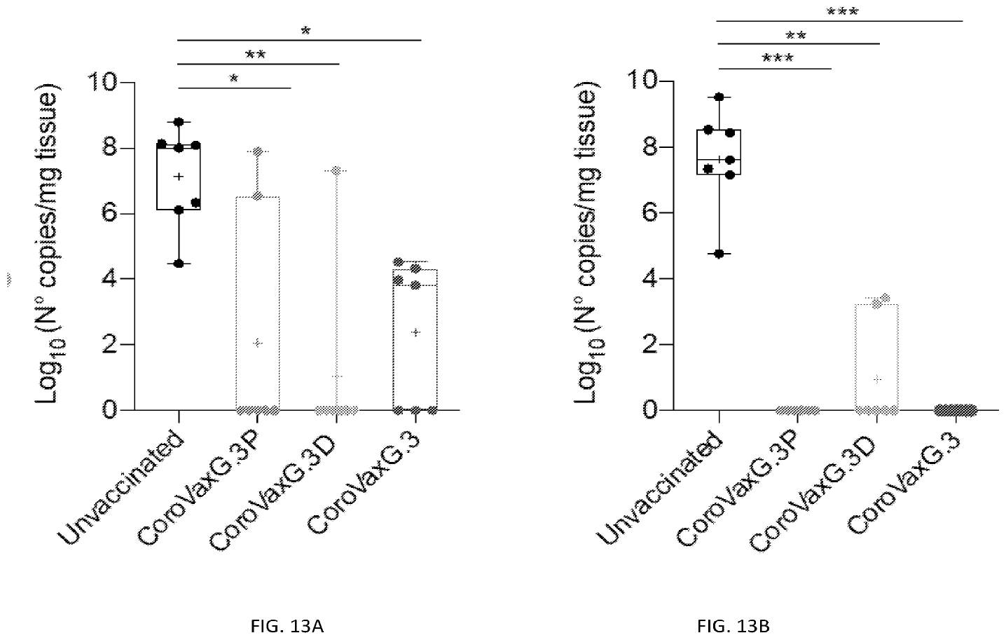

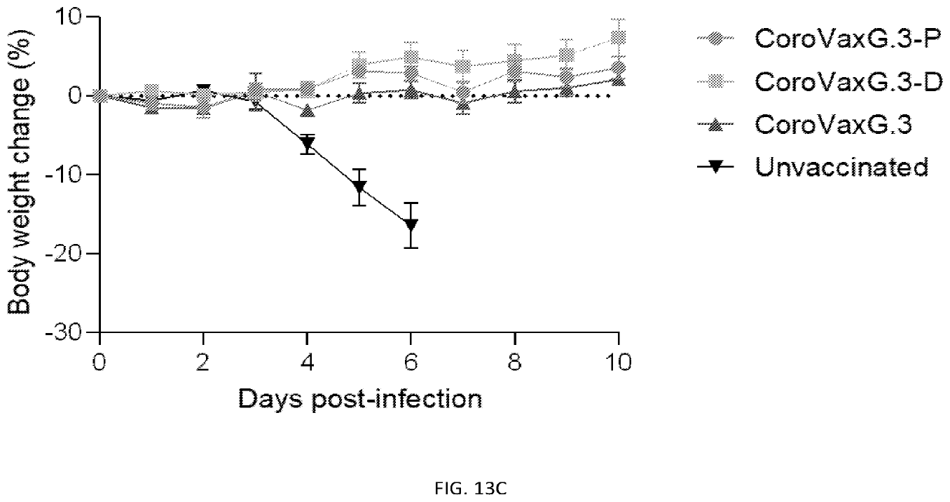

Vaccine protection from a challenge with the VOC Gamma was first investigated. As an initial readout of vaccine protection capacity, viral load in lungs and brain was evaluated at day 4 post-challenge. All unvaccinated control mice showed high viral burden as reflected by the levels of the SARS-CoV-2 Envelop (E) gene both in lungs (FIG. 13A) and brains (FIG. 13B). The high viral burden in lungs and brains was coincidental with body weight loss in the unvaccinated mice (FIG. 13C), and by day 6 all mice of this group had to be euthanized.

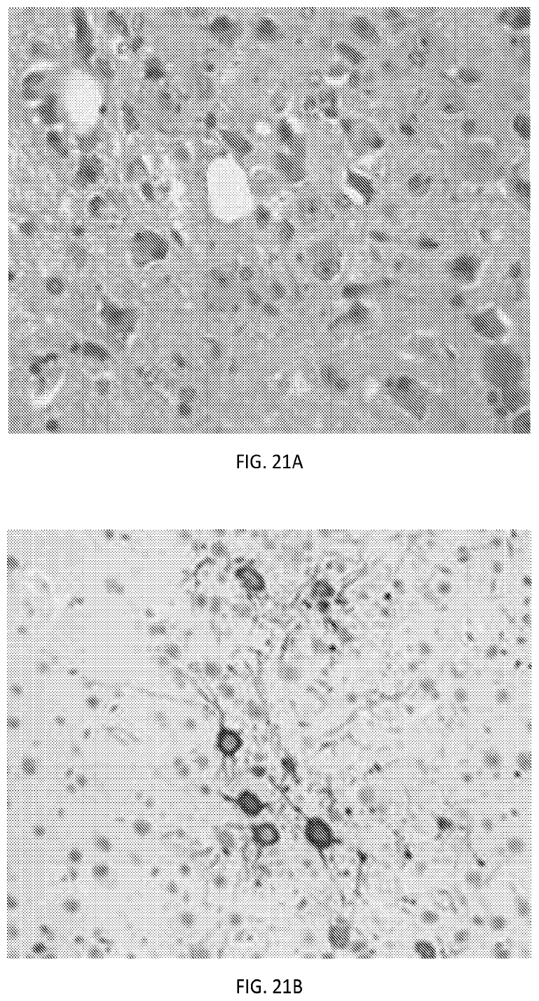

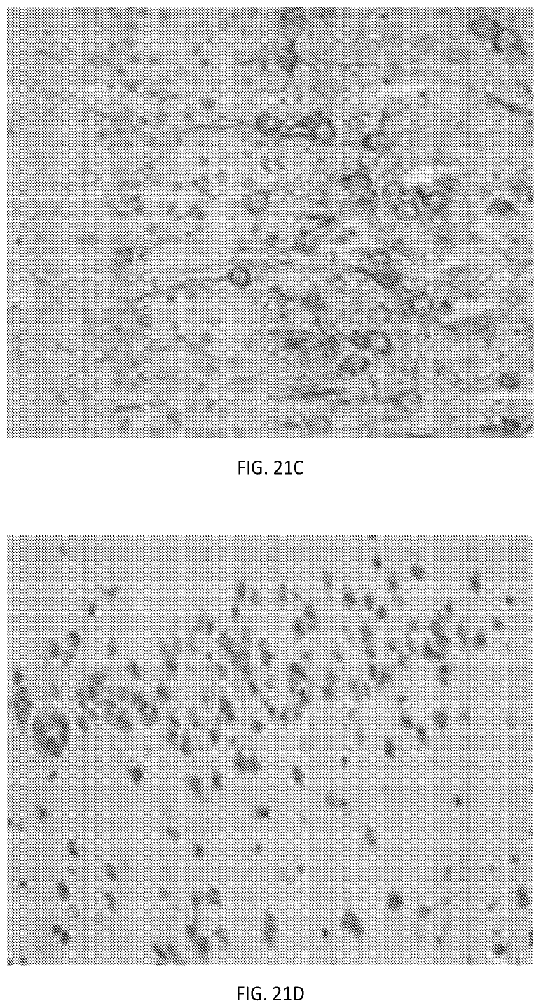

In general, the three vaccines, CoroVaxG.3, CoroVaxG.3-P and CoroVaxG.3-D, conferred robust protection against Gamma infection and replication in affected organs (FIG. 13A and FIG. 13B). Interestingly, protection was much larger for the three vaccines in brain than in lung showing that the three vaccines were able to prevent virus dissemination (FIG. 13B). Moreover, the three vaccines conferred a similar level of protection in terms of mice health; indeed, none of the vaccinated mice lost weight all over the study (FIG. 13C). Histopathological analyses were performed in all organs with particular emphasis on lungs and brains. In close coincidence, histopathological analysis and immunohistochemical assessment for SARS-CoV-2 N protein expression, confirmed that regardless of the vaccine used, all vaccinated mice were completely protected from SARS-CoV-2 dissemination to the brain (FIG. 21A, FIG. 21B, FIG. 21C, and FIG. 21D).

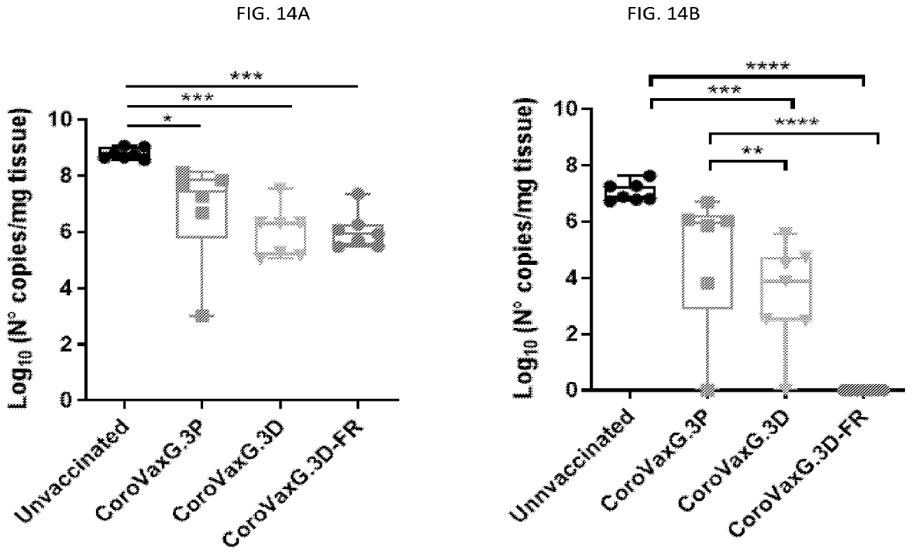

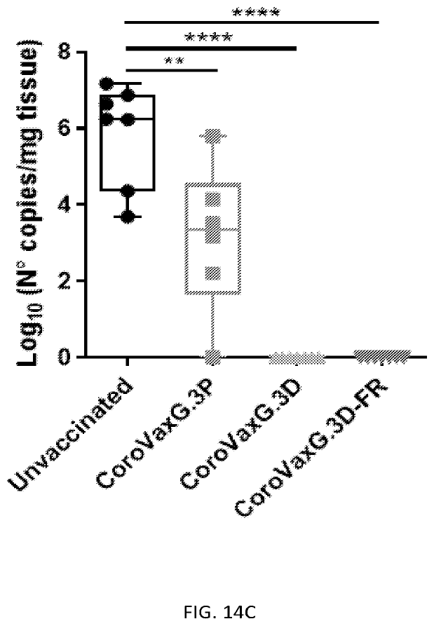

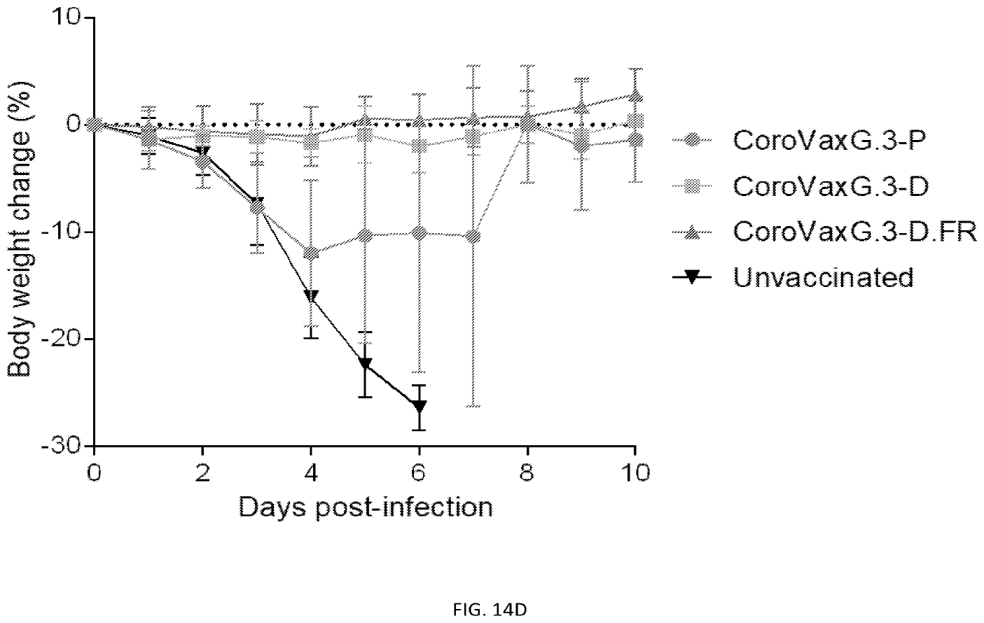

In order to further analyze the capacity of the different VOC-matched vaccines to cross-protect from different SARS-CoV-2 VOC, a similar study was conducted using the Delta strain to challenge vaccinated mice. Since the ancestral strain HU.1 was no longer prevalent in Argentina or globally, mice were immunized with CoroVaxG.3-P and CoroVaxG.3-D and the modified vaccine, CoroVaxG.3-D.FR. Interestingly, clear differences in protection with the different vaccines was observed. Indeed, viral load in lungs was around 1 log lower in mice vaccinated with Delta-matched vaccines compared with the levels in mice vaccinated with the Gamma-matched vaccine (FIG. 14A). Interestingly, analysis of the subgenomic RNA levels showed that vaccination with CoroVaxG.3-D.FR led to the complete inhibition of viral replication in lungs (FIG. 14B). In addition, none of the mice vaccinated with the Delta-matched vaccines exhibited viral replication in brain, while some replication could be still observed in brains of mice vaccinated with the Gamma-matched CoroVaxG.3-P vaccine (FIG. 14C). Thus, the Gamma-matched vaccine was unable to protect mice from non-matched virus dissemination.

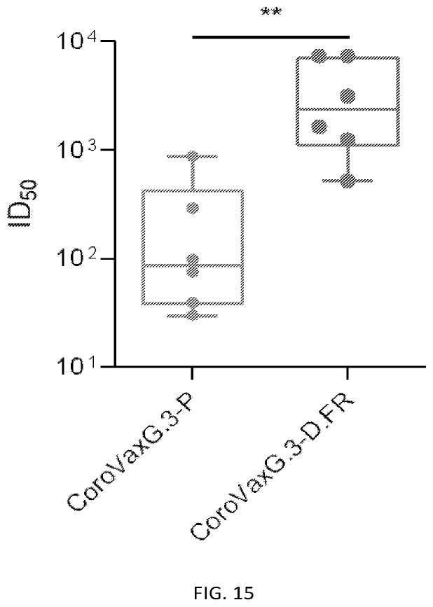

The high viral burden observed in lungs and brains was coincidental with a dramatic weight loss that started at day 3 leading to the euthanasia of all unvaccinated mice by day 6 (FIG. 14D). It was of note that all mice vaccinated with the Gamma-matched vaccine showed weight loss between days 3 to 7. Although most of the mice recovered, 2/7 of mice continued to lose weight leading to their euthanasia by day 6. Contrary to that, all mice vaccinated with the two Delta-matched vaccines showed no weight loss and remained in good health until the end of the study. Consistent with the partial protection from challenge of CoroVaxG.3-P, analysis of the neutralizing antibody titer in sera collected prior to challenge showed that mice vaccinated with CorovaxG.3-D.FR elicited significantly higher levels of anti-SARS-CoV-2 Delta nAbs than animals inoculated with the CorovaxG.3-P candidate (FIG. 15).

### Induction of Omicron-Specific Neutralizing Responses of the Different VOC-Matched CoroVaxG.3 Vaccines

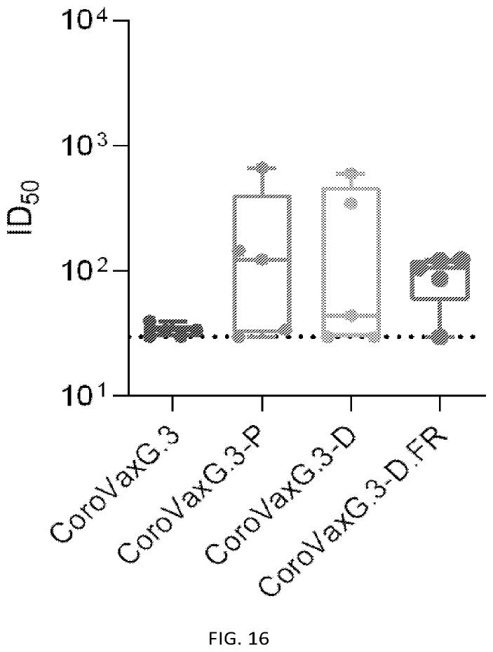

The different CorovaxG.3 vaccine candidates induced a differential cross-neutralization and protection when tested against the VOC Alpha, Beta, Gamma and Delta. During the course of these studies, the VOC Omicron emerged that rapidly outperformed all other VOC. This led to the preparation of a PsV expressing Spike sequence corresponding to Omicron BA.1. Assessment of the neutralizing capacity of sera raised after single-dose vaccination confirmed that no vaccine candidate showed a good level of nAbs against the Omicron BA.1 PsV; although sera obtained from mice vaccinated with Gamma- and Delta-Spike based vaccines induced a slightly better ID50 GMT against Omicron BA.1, than sera from mice vaccinated with the parental CoroVaxG.3 vaccine (FIG. 16). Low ID50 GMT against Omicron was nonetheless expected, since even a vaccine schedule regarded as “complete” for protection of VOC, was no longer sufficient to protect against Omicron. Mean neutralization titers of each vaccine are shown in Table 3.

TABLE 3
Geometric Mean Neutralization Titers of each vaccine candidate
against SARS-CoV-2 Omicron-based pseudoviruses.

| | ID50 GMT |
|---|---|
| | against Omicron |
| Vaccine | (95% CI) |
| CoroVaxG.3 | 33.41 |
| | (28.84-38.71) |
| CoroVaxG.3-P | 108.2 |
| | (22.24-526.7) |
| CoroVaxG.3-D | 96.43 |
| | (16.02-580.6) |
| CoroVaxG.3-D.FR | 84.01 |
| | (40.23-175.4) |

Studies with a Vaccine Against Omicron

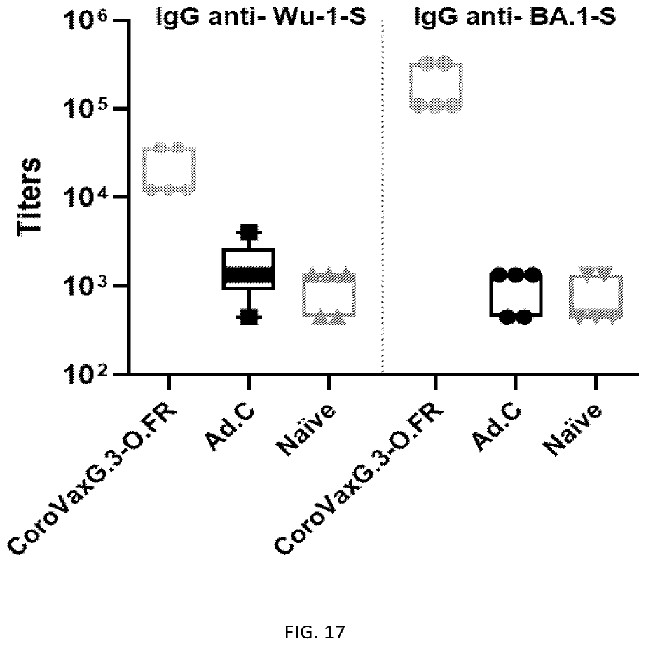

The global healthcare response to the Omicron outbreak was to rapidly administer booster shots, which appeared to work at least to reduce severe stages of the disease¹⁶. Therefore, two different approaches to overcome the evidence that CoroVaxG.3-D.FR was unable to induce Omicron-specific nAbs after one shot administration. In one approach, it was evaluated whether an Omicron-matched vaccine (CoroVaxG.3-O.FR) would confer the necessary protection against its homologous variant. For another approach, the ability of either CoroVaxG.3-D.FR or CoroVaxG.3-O.FR in eliciting Omicron-specific nAbs when administered as a boost to an initial single dose (“complete schedule”) of the vaccine based on the ancestral SARS-CoV-2 spike, CoroVax.G3, was evaluated. As a comparison, a group of mice was administered with the same vaccine, Corovax.G3, as the boost. IgG titers determined by ELISA using Wu-1 Spike as the coating antigen showed a worse performance of the anti-Omicron vaccine as compared to the rest of the candidates (FIG. 17, compare with FIG. 8); however, this was due to the significant alteration of the epitope landscape displayed by Omicron, since this apparent lack of immunogenicity was “restored” once the immunogenic response was tested against Omicron Spike instead of the ancestral Spike (FIG. 17).

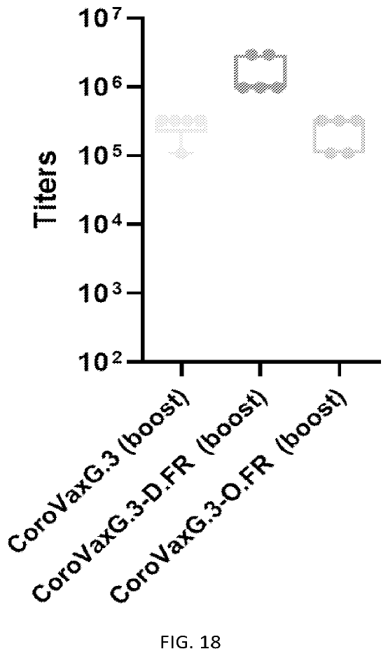

Administration of any of the tested vaccines (CorovaxG.3, CorovaxG.3-D.FR or CorovaxG.3-O.FR) as a boost increased the IgG titers determined against Wu-1 Spike with respect to CorovaxG.3 as a single dose (FIG. 18, compare with FIG. 8).

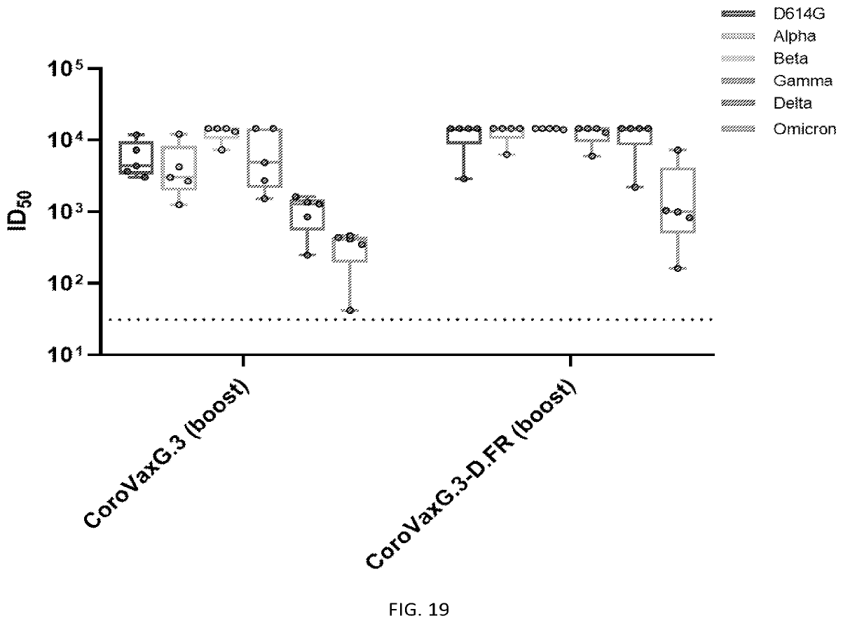

Administration of a booster dose of either vaccine increased the nAb titer against all VOCs, including Omicron. However, a CorovaxG.3-D.FR booster shot was better at increasing the neutralizing capacity of the sera against all variants, which was statistically significant against CorovaxG.3 for neutralization to Delta and Alpha (FIG. 19). For some of the analyzed sera, the calculated ID50 was higher than the maximum assayed dilution of the serum, so lack of statistical significance for other variants may be due to an underestimation of the GMTs.

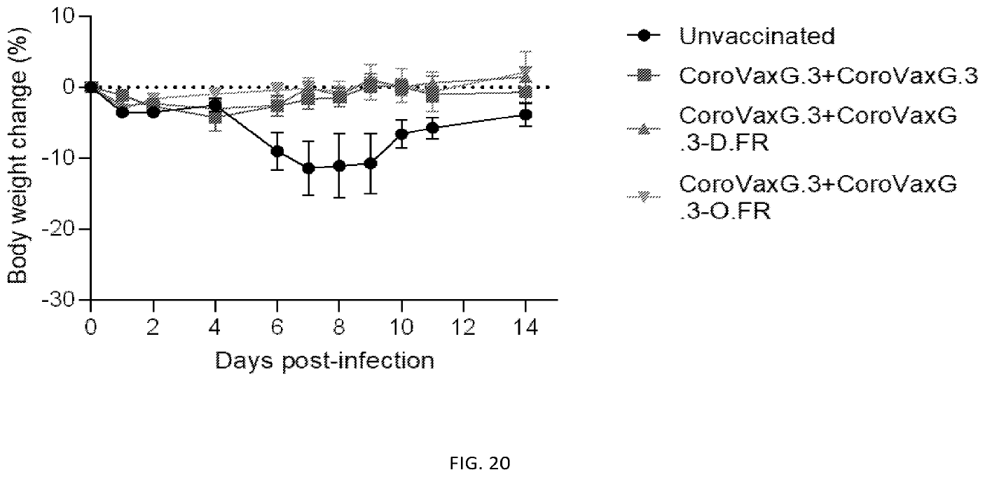

The same prime/boost regime was used to vaccinate K18-hACE2 mice followed by their challenge with the VOC Omicron. After priming mice with a vaccine against the ancestral strain, CoroVaxG.3, mice were boosted with a second dose either of the same vaccine, of CoroVaxG.3-D.FR or CoroVaxG3-O.FR (FIG. 20). Only 2/6 control unvaccinated mice showed no weight loss after a challenge with Omicron. But, 4/6 mice lost more than 10% of body weight euthanasia was required for one of them. On the other hand, only 1/6 mice vaccinated with a booster dose of CoroVaxG.3 lost more than 10% body weight, while none of the mice receiving a booster of CoroVaxG.3-D.FR or CoroVaxG.3-O.FR lost more than 10% weight after a challenge with Omicron (FIG. 20).

### SUMMARY

Currently approved COVID vaccines were designed based on the ancestral Spike protein. The appearance of Variants of Concern (VOC) and sub-variants jeopardized the efficacy of existing vaccines, as these VOC evaded established immunity to different extents. Accordingly, there is a clear need for vaccines with wider protection against multiple COVID variants.

Described herein are adenoviral-based vaccines with wide protection against multiple VOCs. The experiments described herein used preclinical rodent models to establish the cross-neutralizing and cross-protection capacity of adenoviral-based vaccines expressing a membrane bound, prefusion stabilized Spike corresponding to the ancestral B.1 strain, and the VOCs Gamma and Delta. Using specific pseudoviruses (PsV), it was observed that all the vaccines induced high levels of neutralizing antibodies (nAb) against the matched VOC. CorovaxG.3-D.FR, that expresses Delta Spike additionally mutated in the furin cleavage motif, displayed the highest levels of nAb to the matched VOC (Delta) and mismatched strains. Cross-protection against viral infection in lung and dissemination to the brain, organ injury and severe disease in aged K18-hACE2 mice showed dramatic differences among the different vaccines. Indeed, Delta-targeted vaccines were able to protect 100% of mice from a challenge with Gamma; however, an anti-Gamma vaccine offered only a partial protection and 30% of mice succumbed to the challenge with Delta. After a single shot, none of the vaccines was able to induce significant nAbs against Omicron B.1. However, administration of a prime/boost regime showed that a CorovaxG.3-D.FR booster was able to increase the neutralizing capacity of the sera against all variants, including Omicron. Moreover, it was also able to protect 100% of aged K18-hACE2 mice against Omicron as an Omicron-targeted vaccine did. The whole data demonstrate that a booster with CoroVaxG.3-D.FR has the potential to protect from a wide range of SARS-CoV-2 lineages.

### DISCUSSION

By the end of 2020 the SARS-CoV-2-related pandemic entered a new phase with the successive emergence of ‘variants of concern’ with altered viral properties that improved their fitness, leading to an increased capacity to infect ACE-2 expressing cells and evade immune response. After the initial appearance of the B.1.1.7 lineage (VOC Alpha) the emergence and raise of more complex variants such as the B.1.351 (VOC Beta) and P.1 (VOC Gamma) shifted the understanding of the adaptive evolution of SARS-CoV-2. By mid-2021 the lineage B.1.617.2 (VOC Delta) outcompeted previously prevalent variants and became the dominant strain, until the lineage B.1.1.529 (VOC Omicron) was detected in November 2021 and became the prevalent strain only a few months later. The different VOCs showed increased transmissibility and posed risk mainly to unvaccinated individuals and vaccinated people in the elderly or with co-morbidities¹⁷. Despite the fact that most VOCs showed several mutations, with Omicron in particular displaying at least 35 mutations compared to the ancestral strain Wu-H.1, vaccination and administration of booster shots were able to protect people from severe illness reducing deaths to less than 0.1 per 100,000 population during the Delta predominance and Omicron emergence period, compared to almost 8 deaths per 100,000 in unvaccinated individuals¹⁸. Following vaccination, waning from protection against breakthrough infections increased after only few months¹⁹. However, a public health strategy based on vaccinating the population every 4-6 months with a vaccine based on the ancestral Wuhan-Wu-H.1 Spike immunogen does not seem sustainable over time²⁰. Moreover, it is still unclear whether the next influx of infections will come from either sub-variants of Omicron such as BA.4 and BA.5, as it happens currently in different countries, from sub-variants of Delta or Gamma or from unknown variants.

The vaccines described herein induced a comparable response in terms of humoral and cellular immunity and neutralization against a matched PsV, although CoroVaxG.3-F.DR showed statistically significant higher humoral immune response at least compared to CoroVaxG.3 expressing Spike(D614G). Interestingly, the Delta-targeted vaccines, in particular CorovaxG3-D.FR, that presented a non-cleavable Spike in the prefusion conformation, displayed the highest levels of neutralization not only to their matched VOC (Delta) but also to B.1.351 (Beta) and the ancestral B.1. Of note, CoroVaxG.3-D.FR exhibited a much larger ID50 GMT than CoroVaxG.3-D against their matched PsV suggesting that the mutation in the furin-cleavage motif improves antigen presentation and hence vaccine-induced immunity.

All K18-hACE2 transgenic mice vaccinated with any of the vaccines described herein were protected from a challenge with the B.1 strain. All K18-hACE2 transgenic mice were protected from a challenge with the Gamma VOC after vaccination with vaccines expressing Spike derived from the Delta VOC. On the contrary, all the K18-hACE2 transgenic mice vaccinated with CoroVaxG.3-P and challenged with the Delta VOC lost weight and few of them had to be euthanized due to their poor health condition. Thus, the Delta VOC-expressing vaccines showed a better cross protection against a challenge with a non-matched VOC. Interestingly, a booster with CoroVaxG.3-D.FR on mice primed with CoroVaxG.3, was able to protect aged K18-hACE2 mice from a challenge with Omicron similarly to a boost with the Omicron targeted CoroVaxG.3-O.FR vaccine.

Observed viral load was consistent with the fact that the Delta-targeted vaccines cross-protected better against the mismatched Gamma VOC than the opposite. Indeed, only 2/12 mice vaccinated with the Delta Spike-expressing vaccines (regardless of whether they expressed a furin active or mutated domain) showed viral replication in brain after challenge with Gamma; on the contrary, 5/6 K18-hACE2 mice vaccinated with a Gamma Spike-expressing vaccine were unable to block Delta replication in brain, suggesting that a Delta Spike-based vaccine exhibited a broader protection against a mismatched SARS-CoV-2 VOC. Compared to the ancestral Wuhan strain, the Gamma VOC exhibited mutations in the RBD positions N501Y, E484K, and K417T that increased affinity for ACE-2 19-fold (REF); while the Delta VOC bored RBD mutations L452R and T478K that showed only a modest increase in affinity compared with the Wuhan RBD sequence (REF). Despite the differences in the mutations, the structure of the RBD was quite similar among the different VOC with the exception of Omicron²⁴. The fact that the Delta VOC outcompeted the Beta and Gamma VOC and that sera from individuals infected with Gamma were susceptible to a reinfection by Delta²⁵ seems to be related to mutations in the furin cleavage domain rather than differences in the mutations in the RBD/RBM regions. Thus, without wishing to be bound by theory it is possible that nAbs targeted to the furin cleavage domain induced by vaccination with a Delta-targeted vaccine might have a role in improving the cross-protection. In fact, CoroVaxG.3-D.FR that express Delta Spike stabilized in a prefusion conformation in addition to a mutated furin cleavage domain exhibited the most favorable performance among the different vaccine candidates used in this study. And, this vaccine was also able to protect from a challenge with Omicron when administered as a booster.

A significant result of the current studies was the increase in neutralizing capacity of sera from vaccinated mice against matched and mismatched PsVs, including Omicron, following a prime/boost regime; moreover, this prime/boost regime was also able to protect mice from a challenge with mismatched variants as demonstrated for the priming with CoroVaxG.3 followed by a booster with CoroVaxG.3-D.FR and the challenge with Omicron. This clearly suggests that the efficacy of the disclosed vaccines constructs based on retargeted adenoviral vectors expressing high levels of the transgene, is not diminished by the presence of preexisting nAbs. Although it has been widely believed that the pre-existence of nAbs against adenovirus might reduce vaccine efficacy, real world evidence challenged this. Despite the pre-existence of nAbs, 85%-100% of volunteers administered with only one shot of 1.0e11 and 1.5e11 vp of a hAd5V-based vaccine showed seroconversion against SARS-CoV-2^{26, 27}. In the two dose regime of the Astra Zeneca trial, anti-ChAdOx1 nAbs increased with the prime vaccination but not with the boost one; that was in contrast to anti-SARS-CoV-2 nAbs that continued to increase after the boost at 28 days, clearly showing that the pre-existing nAbs against the vector did not preclude the immune response against the transgene²⁸. Of note, hAdV5 is no longer the most prevalent hAd in the world responsible for pediatric and crowded community outbreaks and was replaced by hAdV1, 2, 3, 4, 7 and 14²⁹. Consistent with the evidence of reduced anti-hAdV5 preexisting nAbs in the youngest population preliminary studies in a small cohort of Argentine population shows that less than 20% of people <50 year of age show nAbs against hAdV5; while this number raised to 60% of samples in the population >50 years of age (data not shown). Moreover, preclinical evidence indicates that the annual immunization with the same AdV vector may be effective due to a significant decline in vector immunity³⁰. Real world evidence also shows that AdV-specific T cell response declines with age³¹. It seems then that efficacy induced by adenoviral vectors-based vaccines is not reduced provided enough time between the two doses is granted.

This is the first study that compares the efficacy in terms of neutralizing capacity and protection against a challenge with Gamma, Delta and Omicron VOC of different homologous and heterologous vaccines. Each vaccine was able to induce neutralizing antibodies and protection against the homologous strain. Cross-neutralization and cross-protection studies showed that a single dose of a specific vaccine was not always able to protect against a mismatched VOC, neither to induce nAbs nor inhibit virus dissemination. Consistent with real world evidence and despite the fact that certain VOCs evade established adaptive immunity in vaccinated individuals, a prime/boost regime was able to protect from severe disease regardless of the vaccine used as a booster, included a challenge with Omicron. Moreover, a Delta-targeted vaccine not only induces nAbs against mismatched PsV but could also protect mice from a challenge with Gamma and Omicron.

In sum, the adenoviral vector-based platform described herein, CoroVaxG.3, is able to induce the immunodominance of the transgene. Moreover, this hybrid adenoviral vector can be retargeted to muscle and dendritic cells to increase expression by antigen presenting cells. Thus, the whole data suggest that a booster with CoroVaxG.3-D.FR could provide a long lasting and broad immunization against SARS-CoV-2 strains.

### Materials and Methods

### Reagents and Cells

HEK293T (CRL-3216), Hs 729T (HTB-153) cell lines were obtained from the ATCC (Manassas, VA, USA). HEK293 cells were purchased from Microbix Biosystems Inc (Mississauga, ON, Canada); 911 cells and HEK293T-hACE2 cells were already described¹⁵. All the cell lines were grown in the recommended medium supplemented with 10% of FBS (Natocor, Cordoba, Argentina), 2 mM glutamine, 100 U/mL penicillin and 100 μg/mL streptomycin and maintained in a 37° C. atmosphere containing 5% CO2.

### Vaccines Design and Production

The sequence of the Spike protein gene corresponding to each VOC was extracted from the Outbreak.info database (outbreak.info/situation-reports)³². The sequence of the B.1 ancestral strain was already described¹⁵.

To construct each vaccine version, the plasmid pS-Spike(D614G)-PP (Lopez, 2021), was restricted either with a combination of XhoI/EcoRV or XhoI/SwaI, to delete the Spike sequence to be replaced. Simultaneously, the Spike region was amplified in several overlapping fragments using primers containing the amino-acid changes to be introduced. To build each pShuttle-Spike version, the vector fragment and the Spike fragments were reassembled using Gibson assembly; each pShuttle plasmid version was confirmed by BS-sequencing (CELEMICS, Seul, Korea). To construct the non-replicating adenoviruses, each plasmid pShuttle (plasmid pSSpike) was linearized with PmeI and co-transformed with the E1/E3 deleted and fiber 5/3 adenoviral backbone vector (pAd-ΔE1/E3-F5/3) in electrocompetent BJ5183 bacteria and the positive clones confirmed by sequencing. The recombinant DNA plasmids (pCoro VaxG.3-P, pCoro VaxG.3-D, pCoro VaxG.3-D.FR and pCoro VaxG.3-0) were linearized with PacI and transfected into 911 cells. The rescued vaccines CoroVaxG.3-P, CoroVaxG.3-D, CoroVaxG.3-D.FR and CoroVaxG.3-O.FR were cloned and propagated in HEK-293 cells in CellSTACK® cell culture chambers (Corning, Arizona, USA), purified by double CsCl density gradient centrifugation and stored in 10% glycerol in single-use aliquots at −80° C.

### Assessment of Spike Expression

To assess Spike expression by western blots 1×10⁶ Hs 729T cells were seeded and cultured in 6-well plates overnight. On the other day, cells were transduced with the different adenoviral constructs at MOI 100 for 48 hr. At the end, cells were washed twice with ice-cold PBS and lysed in a 2× Laemmli sample buffer. Protein extracts were separated, transferred to nitrocellulose membranes and probed with an anti-spike Ab (40150-T62, Sino Biological Wayne, PA, USA) and anti-beta-actin Ab (A4700; Sigma, St. Louis, MO, USA). Spike expression was detected and quantified as described¹⁵.

### Mice Immunization

Six- to 8-week-old SPF male BALB/c mice were obtained from the animal facility of the Veterinary School, University of La Plata, Argentina and immunized with 10⁹ viral particles (vp) of Ad.C (empty vector), or each one of the vaccines in 30 μL PBS via i.m. injection in the hind leg. Serum samples were obtained via cardiac puncture of anesthetized mice. The collected whole blood was allowed to clot at 37° C. for 1 h before spinning down at 500×g for 10 min. The clarified sera were stored at −20° C. until use. For booster shots, BALB/c mice were immunized with 10⁹ viral particles (vp) of Corovax.G3. Twenty-eight days later mice were immunized with 10⁹ viral particles (vp) of either Corovax.G3 or Corovax.G3-D.FR. Animals were bled 14 days after the booster dose. Animal studies were carried out following the recommendations of the Guide for the Care and Use of Laboratory Animals of the National Institutes of Health. The protocol was approved by the Institutional Committee for Care and Use of laboratory Animals of the Leloir Institute (CICUAL protocol ID 97).

### ELISA

Animal sera were evaluated for SARS-CoV-2-S-specific IgG antibodies using ELISA. Briefly, ELISA plates (BRANDplates®, immunoGrade, BRAND GMBH+CO KG) were coated with 100 ng of the recombinant SARS-CoV-2 Spike protein (S1+S2 ECD, His-tag, Sino Biological) per well overnight at 4° C. in 50 μL PBS and then blocked with PBS-T/3% BSA (blocking buffer) for 1 h. Following the procedure as described, bound specific IgG was detected with an HRP-conjugated goat anti-mouse IgG H&L antibody (ab6789, Abcam) diluted at 1: 10,000 in a blocking buffer. Color development was performed as described¹⁵.

### IFN-γ Elispot

Spleens were removed from vaccinated or control BALB/c mice at 28 days post immunization, and splenocytes were isolated by disaggregation through a metallic mesh. After RBC lysis with RBC lysis buffer (Biolegend, San Diego, CA, USA), resuspension in RPMI containing 10% FBS and counting, cells were ready for analysis. The IFN-γ-secreting splenocytes were assessed using the ELISPOT mouse IFN-γ kit (R&D Systems, Minneapolis, USA) following manufacturer's protocol. Splenocytes were cultured for 18 h at 5×10⁵ cells per well with 2 μg/mL of a peptide pool consisting mainly of 15-mers (overlapping by 11 amino acids) covering the immunodominant sequence domains of the SARS-CoV-2 Spike protein (PepTivator® SARS-CoV-2 Prot_S; MiltenyiBiotec, Bergisch, Gladbach, Germany). The number of spots was determined using an automatic ELISPOT reader and image analysis software (CTL-ImmunoSpot® S6 MicroAnalyzer, Cellular Technology Limited (CTL), Cleveland, OH, USA).

### Construction of VOC-Matched Pseudoviruses and Neutralization Assays

The full-length cDNA of the different Spike were constructed as follows: to introduce each Spike variant into the pcDNA-3.1, the pcDNA-3.1-Spike¹⁵ was restricted with BamHI/EcoRV or BamHI/SwaI, to delete the section of the Spike sequence to be replaced. Simultaneously, the Spike region was amplified in several overlapping fragments using primers containing the amino-acid changes corresponding to each VOC. Finally, the vector fragment and the Spike fragments were reassembled using Gibson assembly, and confirmed by BS-sequencing (CELEMICS, Seul, Korea). The pseudoviral particles (PVs) containing the different SARS-CoV2 protein variants were generated as described¹⁵. For PsV stocks production, HEK-293T cells were transduced with G*ΔG-VSV followed by transfection with 30 μg of pcDNA-3.1-Spike, washed 4 times with PBS and cultured in complete media at 37° C., 5% CO₂. After 48 h the supernatant containing the PVs was collected, filtered (0.45-μm pore size, Millipore) and stored in single-use aliquots at −80° C.¹⁵.

The 50% tissue culture infectious dose (TCID50) of SARS-CoV-2 PV was determined in sextuplicates and calculated using the Reed-Muench method as previously described³³. The neutralization assays were performed as previously described¹⁵. The percentage of inhibition of infection for each dilution of the sample is calculated according to the RLU values as follows: % inhibition=[1−(average RLU of sample −average RLU of CC)/(average RLU of VC −average RLU of CC)]×100%. The ID50 of each sample was calculated by the Reed-Muench method³³.

In Vivo Challenge Studies with VOC

Heterozygous K18-hACE2 C57BL/6J mice (JAX stock #034860) were obtained from The Jackson Laboratory (Bar Harbor, Maine, US)³⁴⁻³⁶. Animals were bred and housed at the Animal Facility of the Institute of Veterinary Sciences of the National University of the Litoral. Six- to eight-month-old K18-hACE2 mice were immunized with 109 viral particles (vp) of each vaccine in 30 μl PBS via an i.m. injection in the hind leg. For virus challenge, mice were anesthetized (ketamine/xylazine) and infected intranasally with 50 μl containing 1.104 TCID50 of the SARS-CoV-2 Gamma VOC or 5.105 TCID50 of the Delta VOC. The SARS-CoV-2 viral dose selected for each single VOC was selected in preliminary studies and had a similar impact on mice health. Clinical signs of disease (weight loss, rapid breathing, hunched posture and inactivity) were monitored daily until day 14 post-infection or before, if mice reached the endpoint criteria. Lung and brain were harvested at day 4 for viral titer (left half) and histopathological analyses (right half). For the virus challenge studies, K18-hACE2 mice were delivered from the animal facility of the University del Litoral to the Biological Containment Operational Unit of Malbrán Institute, Biosafety Level 3 animal facility. All procedures were performed in strict accordance with the recommendations of the Guide for the Care and Use of Laboratory Animals of the. National Health. Institute. All protocols were approved by the Animal Experimentation Ethics Committee of the Leloir Institute (Protocol ID #94. July 2021, modification November 2021) and were carried out in accordance with the ARRIVE guidelines and the SOPs of the Malbrán Institute. Every effort was made to minimize animal suffering.

Viral Load Assessment Viral titers were determined in lung and brain samples. Tissues were weighed and stored at −80° C. in DMEM. Homogenized tissue was centrifuged 10 min at 2,000 rpm at 4° C. and the supernatant was collected. RNA was extracted with the QIAamp Viral RNA Mini Kit (Qiagen. Germantown, US). SARS-CoV-2 E gene copies, either genomic or subgenomic, were determined by qRT-PCR using LightMix® Modular SARS-CoV (COVID19) kit (TIB MOLBIO) according to the manufacturer's protocol. Results are presented as the log 10 of the number of copies per mg of tissue sample.

### Histopathological Studies

Tissues were collected at necropsy and lung, brain and duodenum samples were fixed in 4% buffered formaldehyde for 8-10 h at room temperature and then washed in PBS. Later, fixed tissues were dehydrated in an ascending series of ethanol, cleared in xylene and embedded in paraffin. Sections (4 μm thick), obtained by rotative microtome, were mounted on slides treated previously with 2% (v/v) 3-aminopropyltriethoxysilane in acetone (Sigma-Aldrich, Saint Louis, MO, USA) and initially stained with hematoxylin-eosin for the histopathology analysis. Deparaffinized slides were used for immunohistochemical staining. Briefly, CD3, but not the SARS-CoV-2 N protein, was retrieved by microwaving in 10 mM sodium citrate buffer (pH 6.0). Endogenous peroxidase activity was inhibited with 3% (v/v) H2O2 in methanol, and nonspecific binding blocked with 10% (v/v) normal goat serum in PBS. The primary antibody against the N SARS-CoV-2 protein (polyclonal rabbit 40143-T62, Sino Biological) were diluted 1:400 in PBS-BSA 1%—Tween 0.5%. The antibody was incubated for 18 h at 4° C. and then for 30 min at room temperature with biotinylated secondary antibodies. Antigens were visualized using the CytoScan HRP Detection System with 3.3-diaminobenzidine (DAB; Liquid DAB-Plus Substrate Kit; Invitrogen) as the chromogen. As a control, adjacent sections were subjected to the same procedure, replacing primary antibodies with rabbit nonimmune sera. Sections were examined by a qualified veterinary pathologist who was blinded to the animal and treatment groups.

### Statistical Analysis

For S-specific binding antibodies as measured by ELISA and ID50s as measured by PBNA, statistical differences between immunization regimens were determined by a Kruskal-Wallis test with Dunn's multiple comparisons a posteriori. All analyses were conducted using GraphPad Prism software (version 8.2). All statistical tests were two-sided, at an overall significance level of α0.05.

For assessment of viral load in Delta studies, comparisons were performed by a ANOVA test, followed by Bonferroni multiple comparisons. For viral load of Gamma comparisons were performed by a non-parametric ANOVA followed by Dunn's test. * p<0.05, **p<0.01, ***p<0.001, **** p<0.0001.

Sequences

SEQ ID NO: 1-Pr 1
TTAATAGTAATCAATTACGGGGTCATTAGTTCATAGCCCATATATGGAGTTCCGCGT
TACATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGACG
TCAATGGGTGGAGTATTTACGGTAAACTGCCCACTTGGCAGTACATCAAGTGTATCA
TATGCCAAGTACGCCCCCTATTGACGTCAATGACGGTAAATGGCCCGCCTGGCATTA
TGCCCAGTACATGACCTTACGGGACTTTCCTACTTGGCAGTACATCTCCACGTTCTGC
TTCACTCTCCCCATCTCCCCCCCCTCCCCACCCCCAATTTTGTATTTATTTATTTTTTA
ATTATTTTGTGCAGCGATGGGGGCGGGGGGGGGGGGGGCGCGCGCCAGGCGGGGCG
GGGCGGGGCGAGGGGCGGGGCGGGGCGAGGCGGAGAGGTGCGGCGGCAGCCAATC
AGAGCGGCGCGCTCCGAAAGTTTCCTTTTATGGCGAGGCGGCGGCGGCGGCGGCCC
TATAAAAAGCGAAGCGCGCGGCGGGGAGTCGCTGCGTTGCCTTCGCCCCGTGCCCC
GCTCCGCGCCGCCTCGCGCCGCCCGCCCCGGCTCTGACTGACCGCGTTACTCCCACA
GGTGAGCGGGCGGGACGGCCCTTCTCCTCCGGGCTGTAATTAGCAAGAGGTAAGGG
TTTAAGGGATGGTTGGTTGGTGGGGTATTAATGTTTA
SEQ ID NO: 2-Pr 2
CGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTG
ACGTCAATGGGTGGAGTATTTACGGTAAACTGCCCACTTGGCAGTACATCAAGTGTA
TCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTA
GTCATCGCTATTACCATGGTCGAGGTGAGCCCCACGTTCTGCTTCACTCTCCCCATCT
CCCCCCCCTCCCCACCCCCAATTTTGTATTTATTTATTTTTTAATTATTTTGTGCAGCG
ATGGGGGCGGGGGGGGGGGGGGGGCGCGCGCCAGGCGGGGCGGGGCGGGGCGAG
GGGCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGA
AGCGCGCGGCGGGCGGGAGTCGCTGCGACGCTGCCTTCGCCCCGTGCCCCGCTCCG
CCGCCGCCTCGCGCCGCCCGCCCCGGCTCTGACTGACCGCGTTACTCCCACAGGTGA
GCGGGCGGGACGGCCCTTCTCCTCCGGGCTGTAATTAGCTGAGCAAGAGGTAAGGG
TTTAAGGGATGGTTGGTTGGTGGGGTATTAATGTTTAATTACCTGGAGCACCTGCCT
GAAATCACTTTTTTTCAGC
SEQ ID NO: 3-Pr 3
TTAATAGTAATCAATTACGGGGTCATTAGTTCATAGCCCATATATGGAGTTCCGCGT
TACATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGACG
TCAATGGGTGGAGTATTTACGGTAAACTGCCCACTTGGCAGTACATCAAGTGTATCA
TATGCCAAGTACGCCCCCTATTGACGTCAATGACGGTAAATGGCCCGCCTGGCATTA
TGCCCAGTACATGACCTTACGGGACTTTCCTACTTGGCAGTACATCTGTCGAGGTGA
GCCCCACGTTCTGCTTCACTCTCCCCATCTCCCCCCCCTCCCCACCCCCAATTTTGTA
TTTATTTATTTTTTAATTATTTTGTGCAGCGATGGGGGCGGGGGGGGGGGGGGGGCG
CGCGCCAGGCGGGGCGGGGCGGGGCGAGGGGCGGGGCGGGGCGAGGCGGAGAGGT
GCGGCGGCAGCCAATCAGAGCGGCGCGCTCCGAAAGTTTCCTTTTATGGCGAGGCG
GCGGCGGCGGCGGCCCTATAAAAAGCGAAGCGCGCGGCGGGCGGGAGTCGCTGCG
TTGCCTTCGCCCCGTGCCCCGCTCCGCGCCGCCTCGCGCCGCCCGCCCCGGCTCTGA
CTGACCGCGTTACTCCCACAGGTGAGCGGGCGGGACGGCCCTTCTCCTCCGGGCTGT
AATTAGCTGAGCAAGAGGTAAGGGTTTAAGGGATGGTTGGTTGGTGGGGTATTAAT
GTTTAATTACCTGGAGCACCTGCCTGAAATCACTTTTTTTCAGC
SEQ ID NO: 4-Pr 4
CGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTG
ACGTCAATGGGTGGAGTATTTACGGTAAACTGCCCACTTGGCAGTACATCAAGTGTA
TCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTA
GTCATCGCTATTACCATGCCACGTTCTGCTTCACTCTCCCCATCTCCCCCCCCTCCCC
ACCCCCAATTTTGTATTTATTTATTTTTTAATTATTTTGTGCAGCGATGGGGGCGGGG
GGGGGGGGGGGGCGCGCCAGGCGGGGCGGGGCGGGGCGAGGGGCGGGGCGGGGC
GAGGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGGGG
AGTCGCTGCGACGCTGCCTTCGCCCCGTGCCCCGCTCCGCCGCCGCCTCGCGCCGCC
CGCCCCGGCTCTGACTGACCGCGTTACTCCCACAGGTGAGCGGGCGGGACGGCCCTT
CTCCTCCGGGCTGTAATTAGCTGAGCAAGAGGTAAGGGTTTAAGGGATGGTTGGTTG
GTGGGGTATTAATGTTTAATTACCTGGAGCACCTGCCTGAAATCACTTTTTTTCAGC
SEQ ID NO: 5-Spike D614G-WPRE-polyA
ATGTTCGTGTTCCTGGTGCTGCTGCCCCTGGTGAGCAGCCAGTGCGTGAACCTGACC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGACAACCCCGTGCTGCCCTTCAACGACGGCGTGTACTTCGCCAGCA
CCGAGAAGAGCAACATCATCAGAGGCTGGATCTTCGGCACCACCCTGGACAGCAAG
ACCCAGAGCCTGCTGATCGTGAACAACGCCACCAACGTGGTGATCAAGGTGTGCGA
GTTCCAGTTCTGCAACGACCCCTTCCTGGGCGTGTACTACCACAAGAACAACAAGAG
CTGGATGGAGAGCGAGTTCAGAGTGTACAGCAGCGCCAACAACTGCACCTTCGAGT
ACGTGAGCCAGCCCTTCCTGATGGACCTGGAGGGCAAGCAGGGCAACTTCAAGAAC
CTGAGAGAGTTCGTGTTCAAGAACATCGACGGCTACTTCAAGATCTACAGCAAGCA
CACCCCCATCAACCTGGTGAGAGACCTGCCCCAGGGCTTCAGCGCCCTGGAGCCCCT
GGTGGACCTGCCCATCGGCATCAACATCACCAGATTCCAGACCCTGCTGGCCCTGCA
CAGAAGCTACCTGACCCCCGGCGACAGCAGCAGCGGCTGGACCGCCGGCGCCGCCG
CCTACTACGTGGGCTACCTGCAGCCCAGAACCTTCCTGCTGAAGTACAACGAGAAC
GGCACCATCACCGACGCCGTGGACTGCGCCCTGGACCCCCTGAGCGAGACCAAGTG
CACCCTGAAGAGCTTCACCGTGGAGAAGGGCATCTACCAGACCAGCAACTTCAGAG
TGCAGCCCACCGAGAGCATCGTGAGATTCCCCAACATCACCAACCTGTGCCCCTTCG
GCGAGGTGTTCAACGCCACCAGATTCGCCAGCGTGTACGCCTGGAACAGAAAGAGA
ATCAGCAACTGCGTGGCCGACTACAGCGTGCTGTACAACAGCGCCAGCTTCAGCAC
CTTCAAGTGCTACGGCGTGAGCCCCACCAAGCTGAACGACCTGTGCTTCACCAACGT
GTACGCCGACAGCTTCGTGATCAGAGGCGACGAGGTGAGACAGATCGCCCCCGGCC
AGACCGGCAAGATCGCCGACTACAACTACAAGCTGCCCGACGACTTCACCGGCTGC
GTGATCGCCTGGAACAGCAACAACCTGGACAGCAAGGTGGGCGGCAACTACAACTA
CCTGTACAGACTGTTCAGAAAGAGCAACCTGAAGCCCTTCGAGAGAGACATCAGCA
CCGAGATCTACCAGGCCGGCAGCACCCCCTGCAACGGCGTGGAGGGCTTCAACTGC
TACTTCCCCCTGCAGAGCTACGGCTTCCAGCCCACCAACGGCGTGGGCTACCAGCCC
TACAGAGTGGTGGTGCTGAGCTTCGAGCTGCTGCACGCCCCCGCCACCGTGTGCGGC
CCCAAGAAGAGCACCAACCTGGTGAAGAACAAGTGCGTGAACTTCAACTTCAACGG
CCTGACCGGCACCGGCGTGCTGACCGAGAGCAACAAGAAGTTCCTGCCCTTCCAGC
AGTTCGGCAGAGACATCGCCGACACCACCGACGCCGTGAGAGACCCCCAGACCCTG
GAGATCCTGGACATCACCCCCTGCAGCTTCGGCGGCGTGAGCGTGATCACCCCCGGC
ACCAACACCAGCAACCAGGTGGCCGTGCTGTACCAGGGCGTGAACTGCACCGAGGT
GCCCGTGGCCATCCACGCCGACCAGCTGACCCCCACCTGGAGAGTGTACAGCACCG
GCAGCAACGTGTTCCAGACCAGAGCCGGCTGCCTGATCGGCGCCGAGCACGTGAAC
AACAGCTACGAGTGCGACATCCCCATCGGCGCCGGCATCTGCGCCAGCTACCAGAC
CCAGACCAACAGCCCCAGAAGAGCCAGAAGCGTGGCCAGCCAGAGCATCATCGCCT
ACACCATGAGCCTGGGCGCCGAGAACAGCGTGGCCTACAGCAACAACAGCATCGCC
ATCCCCACCAACTTCACCATCAGCGTGACCACCGAGATCCTGCCCGTGAGCATGACC
AAGACCAGCGTGGACTGCACCATGTACATCTGCGGCGACAGCACCGAGTGCAGCAA
CCTGCTGCTGCAGTACGGCAGCTTCTGCACCCAGCTGAACAGAGCCCTGACCGGCAT
CGCCGTGGAGCAGGACAAGAACACCCAGGAGGTGTTCGCCCAGGTGAAGCAGATCT
ACAAGACCCCCCCCATCAAGGACTTCGGCGGCTTCAACTTCAGCCAGATCCTGCCCG
ACCCCAGCAAGCCCAGCAAGAGAAGCTTCATCGAGGACCTGCTGTTCAACAAGGTG
ACCCTGGCCGACGCCGGCTTCATCAAGCAGTACGGCGACTGCCTGGGCGACATCGC
CGCCAGAGACCTGATCTGCGCCCAGAAGTTCAACGGCCTGACCGTGCTGCCCCCCCT
GCTGACCGACGAGATGATCGCCCAGTACACCAGCGCCCTGCTGGCCGGCACCATCA
CCAGCGGCTGGACCTTCGGCGCCGGCGCCGCCCTGCAGATCCCCTTCGCCATGCAGA
TGGCCTACAGATTCAACGGCATCGGCGTGACCCAGAACGTGCTGTACGAGAACCAG
AAGCTGATCGCCAACCAGTTCAACAGCGCCATCGGCAAGATCCAGGACAGCCTGAG
CAGCACCGCCAGCGCCCTGGGCAAGCTGCAGGACGTGGTGAACCAGAACGCCCAGG
CCCTGAACACCCTGGTGAAGCAGCTGAGCAGCAACTTCGGCGCCATCAGCAGCGTG
CTGAACGATATCCTGAGCAGACTGGACAAGGTGGAGGCCGAGGTGCAGATCGACAG
ACTGATCACCGGCAGACTGCAGAGCCTGCAGACCTACGTGACCCAGCAGCTGATCA
GAGCCGCCGAGATCAGAGCCAGCGCCAACCTGGCCGCCACCAAGATGAGCGAGTGC
GTGCTGGGCCAGAGCAAGAGAGTGGACTTCTGCGGCAAGGGCTACCACCTGATGAG
CTTCCCCCAGAGCGCCCCCCACGGCGTGGTGTTCCTGCACGTGACCTACGTGCCCGC
CCAGGAGAAGAACTTCACCACCGCCCCCGCCATCTGCCACGACGGCAAGGCCCACT
TCCCCAGAGAGGGCGTGTTCGTGAGCAACGGCACCCACTGGTTCGTGACCCAGAGA
AACTTCTACGAGCCCCAGATCATCACCACCGACAACACCTTCGTGAGCGGCAACTGC
GACGTGGTGATCGGCATCGTGAACAACACCGTGTACGACCCCCTGCAGCCCGAGCT
GGACAGCTTCAAGGAGGAGCTGGACAAGTACTTCAAGAACCACACCAGCCCCGACG
TGGACCTGGGCGACATCAGCGGCATCAACGCCAGCGTGGTGAACATCCAGAAGGAG
ATCGACAGACTGAACGAGGTGGCCAAGAATTTAAATGAGAGCCTGATCGACCTGCA
GGAGCTGGGCAAGTACGAGCAGTACATCAAGTGGCCCTGGTACATCTGGCTGGGCT
TCATCGCCGGCCTGATCGCCATCGTGATGGTGACCATCATGCTGTGCTGCATGACCA
GCTGCTGCAGCTGCCTGAAGGGCTGCTGCAGCTGCGGCAGCTGCTGCAAGTTCGAC
GAGGACGACAGCGAGCCCGTGCTGAAGGGCGTGAAGCTGCACTACACCTAAACTAG
AAATCAACCTCTGGATTACAAAATTTGTGAAAGATTGACTGGTATTCTTAACTATGT
TGCTCCTTTTACGCTATGTGGATACGCTGCTTTAATGCCTTTGTATCATGCTATTGCTT
CCCGTATGGCTTTCATTTTCTCCTCCTTGTATAAATCCTGGTTGCTGTCTCTTTATGAG
GAGTTGTGGCCCGTTGTCAGGCAACGTGGCGTGGTGTGCACTGTGTTTGCTGACGCA
ACCCCCACTGGTTGGGGCATTGCCACCACCTGTCAGCTCCTTTCCGGGACTTTCGCTT
TCCCCCTCCCTATTGCCACGGCGGAACTCATCGCCGCCTGCCTTGCCCGCTGCTGGA
CAGGGGCTCGGCTGTTGGGCACTGACAATTCCGTGGTGTTGTCGGGGAAATCATCGT
CCTTTCCTTGGCTGCTCGCCTGTGTTGCCACCTGGATTCTGCGCGGGACGTCCTTCTG
CTACGTCCCTTCGGCCCTCAATCCAGCGGACCTTCCTTCCCGCGGCCTGCTGCCGGCT
CTGCGGCCTCTTCCGCGTCTTCGCCTTCGCCCTCAGACGAGTCGGATCTCCCTTTGGG
CCGCCTCCCCGCCAGACATGATAAGATACATTGATGAGTTTGGACAAACCACAACT
AGAATGCAGTGAAAAAAATGCTTTATTTGTGAAATTTGTGATGCTATTGCTTTATTTG
TAACCATTATAAGCTGCAATAAACAAGTTAACAACAACAATTGCATTCATTTTATGT
TTCAGGTTCAGGGGGAGGTGTGGGAGGTTTTTTAAAGCAAGTAAAACCTCTACAAAT
GTGGTA
SEQ ID NO: 6-Spike D614G-IRES-hCD40L-WPRE-polyA
ATGTTCGTGTTCCTGGTGCTGCTGCCCCTGGTGAGCAGCCAGTGCGTGAACCTGACC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGACAACCCCGTGCTGCCCTTCAACGACGGCGTGTACTTCGCCAGCA
CCGAGAAGAGCAACATCATCAGAGGCTGGATCTTCGGCACCACCCTGGACAGCAAG
ACCCAGAGCCTGCTGATCGTGAACAACGCCACCAACGTGGTGATCAAGGTGTGCGA
GTTCCAGTTCTGCAACGACCCCTTCCTGGGCGTGTACTACCACAAGAACAACAAGAG
CTGGATGGAGAGCGAGTTCAGAGTGTACAGCAGCGCCAACAACTGCACCTTCGAGT
ACGTGAGCCAGCCCTTCCTGATGGACCTGGAGGGCAAGCAGGGCAACTTCAAGAAC
CTGAGAGAGTTCGTGTTCAAGAACATCGACGGCTACTTCAAGATCTACAGCAAGCA
CACCCCCATCAACCTGGTGAGAGACCTGCCCCAGGGCTTCAGCGCCCTGGAGCCCCT
GGTGGACCTGCCCATCGGCATCAACATCACCAGATTCCAGACCCTGCTGGCCCTGCA
CAGAAGCTACCTGACCCCCGGCGACAGCAGCAGCGGCTGGACCGCCGGCGCCGCCG
CCTACTACGTGGGCTACCTGCAGCCCAGAACCTTCCTGCTGAAGTACAACGAGAAC
GGCACCATCACCGACGCCGTGGACTGCGCCCTGGACCCCCTGAGCGAGACCAAGTG
CACCCTGAAGAGCTTCACCGTGGAGAAGGGCATCTACCAGACCAGCAACTTCAGAG
TGCAGCCCACCGAGAGCATCGTGAGATTCCCCAACATCACCAACCTGTGCCCCTTCG
GCGAGGTGTTCAACGCCACCAGATTCGCCAGCGTGTACGCCTGGAACAGAAAGAGA
ATCAGCAACTGCGTGGCCGACTACAGCGTGCTGTACAACAGCGCCAGCTTCAGCAC
CTTCAAGTGCTACGGCGTGAGCCCCACCAAGCTGAACGACCTGTGCTTCACCAACGT
GTACGCCGACAGCTTCGTGATCAGAGGCGACGAGGTGAGACAGATCGCCCCCGGCC
AGACCGGCAAGATCGCCGACTACAACTACAAGCTGCCCGACGACTTCACCGGCTGC
GTGATCGCCTGGAACAGCAACAACCTGGACAGCAAGGTGGGCGGCAACTACAACTA
CCTGTACAGACTGTTCAGAAAGAGCAACCTGAAGCCCTTCGAGAGAGACATCAGCA
CCGAGATCTACCAGGCCGGCAGCACCCCCTGCAACGGCGTGGAGGGCTTCAACTGC
TACTTCCCCCTGCAGAGCTACGGCTTCCAGCCCACCAACGGCGTGGGCTACCAGCCC
TACAGAGTGGTGGTGCTGAGCTTCGAGCTGCTGCACGCCCCCGCCACCGTGTGCGGC
CCCAAGAAGAGCACCAACCTGGTGAAGAACAAGTGCGTGAACTTCAACTTCAACGG
CCTGACCGGCACCGGCGTGCTGACCGAGAGCAACAAGAAGTTCCTGCCCTTCCAGC
AGTTCGGCAGAGACATCGCCGACACCACCGACGCCGTGAGAGACCCCCAGACCCTG
GAGATCCTGGACATCACCCCCTGCAGCTTCGGCGGCGTGAGCGTGATCACCCCCGGC
ACCAACACCAGCAACCAGGTGGCCGTGCTGTACCAGGGCGTGAACTGCACCGAGGT
GCCCGTGGCCATCCACGCCGACCAGCTGACCCCCACCTGGAGAGTGTACAGCACCG
GCAGCAACGTGTTCCAGACCAGAGCCGGCTGCCTGATCGGCGCCGAGCACGTGAAC
AACAGCTACGAGTGCGACATCCCCATCGGCGCCGGCATCTGCGCCAGCTACCAGAC
CCAGACCAACAGCCCCAGAAGAGCCAGAAGCGTGGCCAGCCAGAGCATCATCGCCT
ACACCATGAGCCTGGGCGCCGAGAACAGCGTGGCCTACAGCAACAACAGCATCGCC
ATCCCCACCAACTTCACCATCAGCGTGACCACCGAGATCCTGCCCGTGAGCATGACC
AAGACCAGCGTGGACTGCACCATGTACATCTGCGGCGACAGCACCGAGTGCAGCAA
CCTGCTGCTGCAGTACGGCAGCTTCTGCACCCAGCTGAACAGAGCCCTGACCGGCAT
CGCCGTGGAGCAGGACAAGAACACCCAGGAGGTGTTCGCCCAGGTGAAGCAGATCT
ACAAGACCCCCCCCATCAAGGACTTCGGCGGCTTCAACTTCAGCCAGATCCTGCCCG
ACCCCAGCAAGCCCAGCAAGAGAAGCTTCATCGAGGACCTGCTGTTCAACAAGGTG
ACCCTGGCCGACGCCGGCTTCATCAAGCAGTACGGCGACTGCCTGGGCGACATCGC
CGCCAGAGACCTGATCTGCGCCCAGAAGTTCAACGGCCTGACCGTGCTGCCCCCCCT
GCTGACCGACGAGATGATCGCCCAGTACACCAGCGCCCTGCTGGCCGGCACCATCA
CCAGCGGCTGGACCTTCGGCGCCGGCGCCGCCCTGCAGATCCCCTTCGCCATGCAGA
TGGCCTACAGATTCAACGGCATCGGCGTGACCCAGAACGTGCTGTACGAGAACCAG
AAGCTGATCGCCAACCAGTTCAACAGCGCCATCGGCAAGATCCAGGACAGCCTGAG
CAGCACCGCCAGCGCCCTGGGCAAGCTGCAGGACGTGGTGAACCAGAACGCCCAGG
CCCTGAACACCCTGGTGAAGCAGCTGAGCAGCAACTTCGGCGCCATCAGCAGCGTG
CTGAACGATATCCTGAGCAGACTGGACAAGGTGGAGGCCGAGGTGCAGATCGACAG
ACTGATCACCGGCAGACTGCAGAGCCTGCAGACCTACGTGACCCAGCAGCTGATCA
GAGCCGCCGAGATCAGAGCCAGCGCCAACCTGGCCGCCACCAAGATGAGCGAGTGC
GTGCTGGGCCAGAGCAAGAGAGTGGACTTCTGCGGCAAGGGCTACCACCTGATGAG
CTTCCCCCAGAGCGCCCCCCACGGCGTGGTGTTCCTGCACGTGACCTACGTGCCCGC
CCAGGAGAAGAACTTCACCACCGCCCCCGCCATCTGCCACGACGGCAAGGCCCACT
TCCCCAGAGAGGGCGTGTTCGTGAGCAACGGCACCCACTGGTTCGTGACCCAGAGA
AACTTCTACGAGCCCCAGATCATCACCACCGACAACACCTTCGTGAGCGGCAACTGC
GACGTGGTGATCGGCATCGTGAACAACACCGTGTACGACCCCCTGCAGCCCGAGCT
GGACAGCTTCAAGGAGGAGCTGGACAAGTACTTCAAGAACCACACCAGCCCCGACG
TGGACCTGGGCGACATCAGCGGCATCAACGCCAGCGTGGTGAACATCCAGAAGGAG
ATCGACAGACTGAACGAGGTGGCCAAGAATTTAAATGAGAGCCTGATCGACCTGCA
GGAGCTGGGCAAGTACGAGCAGTACATCAAGTGGCCCTGGTACATCTGGCTGGGCT
TCATCGCCGGCCTGATCGCCATCGTGATGGTGACCATCATGCTGTGCTGCATGACCA
GCTGCTGCAGCTGCCTGAAGGGCTGCTGCAGCTGCGGCAGCTGCTGCAAGTTCGAC
GAGGACGACAGCGAGCCCGTGCTGAAGGGCGTGAAGCTGCACTACACCTAAACTAG
TCGCCCCTCTCCCTCCCCCCCCCCTAACGTTACTGGCCGAAGCCGCTTGGAATAAGG
CCGGTGTGCGTTTGTCTATATGTTATTTTCCACCATATTGCCGTCTTTTGGCAATGTG
AGGGCCCGGAAACCTGGCCCTGTCTTCTTGACGAGCATTCCTAGGGGTCTTTCCCCT
CTCGCCAAAGGAATGCAAGGTCTGTTGAATGTCGTGAAGGAAGCAGTTCCTCTGGA
AGCTTCTTGAAGACAAACAACGTCTGTAGCGACCCTTTGCAGGCAGCGGAACCCCC
CACCTGGCGACAGGTGCCTCTGCGGCCAAAAGCCACGTGTATAAGATACACCTGCA
AAGGCGGCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGGTACCCC
ATTGTATGGGATCTGATCTGGGGCCTCGGTGCACATGCTTTACATGTGTTTAGTCGA
GGTTAAAAAACGTCTAGGCCCCCCGAACCACGGGGACGTGGTTTTCCTTTGAAAAAC
ACGATGATAATATGATCGAAACATACAACCAAACTTCTCCCCGATCTGCGGCCACTG
GACTGCCCATCAGCATGAAAATTTTTATGTATCTCCTTACTGTTTTTCTTATCACCCA
GATGATTGGGTCAGCACTTTTTGCTGTGTATCTTCATAGAAGGTTGGACAAGATAGA
AGATGAAAGGAATCTTCATGAAGATTTTGTATTCATGAAAACGATACAGAGATGCA
ACACAGGAGAAAGATCCTTATCCTTACTGAACTGTGAGGAGATCAAAAGCCAGTTT
GAAGGCTTTGTGAAGGATATAATGTTAAACAAAGAGGAGACGAAGAAAGAAAACA
GCGGAGATCAGAATCCTCAAATTGCGGCACATGTCATAAGTGAGGCCAGCAGTAAA
ACAACATCTGTGTTACAGTGGGCTGAAAAAGGATACTACACCATGAGCAACAACTT
GGTAACCCTGGAAAATGGGAAACAGCTGACCGTTAAAAGACAAGGACTCTATTATA
TCTATGCCCAAGTCACCTTCTGTTCCAATCGGGAAGCTTCGAGTCAAGCTCCCTTTAT
AGCCAGCCTCTGCCTAAAGTCCCCCGGTAGATTCGAGAGAATCTTACTCAGAGCTGC
AAATACCCACAGTTCCGCCAAACCTTGCGGGCAACAATCCATTCACTTGGGAGGAG
TATTTGAATTGCAACCAGGTGCTTCGGTGTTTGTCAATGTGACTGATCCAAGCCAAG
TGAGCCATGGCACTGGCTTCACGTCCTTTGGCTTACTCAAACTCTGATCTAGAAATC
AACCTCTGGATTACAAAATTTGTGAAAGATTGACTGGTATTCTTAACTATGTTGCTCC
TTTTACGCTATGTGGATACGCTGCTTTAATGCCTTTGTATCATGCTATTGCTTCCCGT
ATGGCTTTCATTTTCTCCTCCTTGTATAAATCCTGGTTGCTGTCTCTTTATGAGGAGTT
GTGGCCCGTTGTCAGGCAACGTGGCGTGGTGTGCACTGTGTTTGCTGACGCAACCCC
CACTGGTTGGGGCATTGCCACCACCTGTCAGCTCCTTTCCGGGACTTTCGCTTTCCCC
CTCCCTATTGCCACGGCGGAACTCATCGCCGCCTGCCTTGCCCGCTGCTGGACAGGG
GCTCGGCTGTTGGGCACTGACAATTCCGTGGTGTTGTCGGGGAAATCATCGTCCTTT
CCTTGGCTGCTCGCCTGTGTTGCCACCTGGATTCTGCGCGGGACGTCCTTCTGCTACG
TCCCTTCGGCCCTCAATCCAGCGGACCTTCCTTCCCGCGGCCTGCTGCCGGCTCTGCG
GCCTCTTCCGCGTCTTCGCCTTCGCCCTCAGACGAGTCGGATCTCCCTTTGGGCCGCC
TCCCCGCCAGACATGATAAGATACATTGATGAGTTTGGACAAACCACAACTAGAAT
GCAGTGAAAAAAATGCTTTATTTGTGAAATTTGTGATGCTATTGCTTTATTTGTAACC
ATTATAAGCTGCAATAAACAAGTTAACAACAACAATTGCATTCATTTTATGTTTCAG
GTTCAGGGGGAGGTGTGGGAGGTTTTTTAAAGCAAGTAAAACCTCTACAAATGTGG
TA
SEQ ID NO: 7-Spike D614G-PP-WPRE-polyA
ATGTTCGTGTTCCTGGTGCTGCTGCCCCTGGTGAGCAGCCAGTGCGTGAACCTGACC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGACAACCCCGTGCTGCCCTTCAACGACGGCGTGTACTTCGCCAGCA
CCGAGAAGAGCAACATCATCAGAGGCTGGATCTTCGGCACCACCCTGGACAGCAAG
ACCCAGAGCCTGCTGATCGTGAACAACGCCACCAACGTGGTGATCAAGGTGTGCGA
GTTCCAGTTCTGCAACGACCCCTTCCTGGGCGTGTACTACCACAAGAACAACAAGAG
CTGGATGGAGAGCGAGTTCAGAGTGTACAGCAGCGCCAACAACTGCACCTTCGAGT
ACGTGAGCCAGCCCTTCCTGATGGACCTGGAGGGCAAGCAGGGCAACTTCAAGAAC
CTGAGAGAGTTCGTGTTCAAGAACATCGACGGCTACTTCAAGATCTACAGCAAGCA
CACCCCCATCAACCTGGTGAGAGACCTGCCCCAGGGCTTCAGCGCCCTGGAGCCCCT
GGTGGACCTGCCCATCGGCATCAACATCACCAGATTCCAGACCCTGCTGGCCCTGCA
CAGAAGCTACCTGACCCCCGGCGACAGCAGCAGCGGCTGGACCGCCGGCGCCGCCG
CCTACTACGTGGGCTACCTGCAGCCCAGAACCTTCCTGCTGAAGTACAACGAGAAC
GGCACCATCACCGACGCCGTGGACTGCGCCCTGGACCCCCTGAGCGAGACCAAGTG
CACCCTGAAGAGCTTCACCGTGGAGAAGGGCATCTACCAGACCAGCAACTTCAGAG
TGCAGCCCACCGAGAGCATCGTGAGATTCCCCAACATCACCAACCTGTGCCCCTTCG
GCGAGGTGTTCAACGCCACCAGATTCGCCAGCGTGTACGCCTGGAACAGAAAGAGA
ATCAGCAACTGCGTGGCCGACTACAGCGTGCTGTACAACAGCGCCAGCTTCAGCAC
CTTCAAGTGCTACGGCGTGAGCCCCACCAAGCTGAACGACCTGTGCTTCACCAACGT
GTACGCCGACAGCTTCGTGATCAGAGGCGACGAGGTGAGACAGATCGCCCCCGGCC
AGACCGGCAAGATCGCCGACTACAACTACAAGCTGCCCGACGACTTCACCGGCTGC
GTGATCGCCTGGAACAGCAACAACCTGGACAGCAAGGTGGGCGGCAACTACAACTA
CCTGTACAGACTGTTCAGAAAGAGCAACCTGAAGCCCTTCGAGAGAGACATCAGCA
CCGAGATCTACCAGGCCGGCAGCACCCCCTGCAACGGCGTGGAGGGCTTCAACTGC
TACTTCCCCCTGCAGAGCTACGGCTTCCAGCCCACCAACGGCGTGGGCTACCAGCCC
TACAGAGTGGTGGTGCTGAGCTTCGAGCTGCTGCACGCCCCCGCCACCGTGTGCGGC
CCCAAGAAGAGCACCAACCTGGTGAAGAACAAGTGCGTGAACTTCAACTTCAACGG
CCTGACCGGCACCGGCGTGCTGACCGAGAGCAACAAGAAGTTCCTGCCCTTCCAGC
AGTTCGGCAGAGACATCGCCGACACCACCGACGCCGTGAGAGACCCCCAGACCCTG
GAGATCCTGGACATCACCCCCTGCAGCTTCGGCGGCGTGAGCGTGATCACCCCCGGC
ACCAACACCAGCAACCAGGTGGCCGTGCTGTACCAGGGCGTGAACTGCACCGAGGT
GCCCGTGGCCATCCACGCCGACCAGCTGACCCCCACCTGGAGAGTGTACAGCACCG
GCAGCAACGTGTTCCAGACCAGAGCCGGCTGCCTGATCGGCGCCGAGCACGTGAAC
AACAGCTACGAGTGCGACATCCCCATCGGCGCCGGCATCTGCGCCAGCTACCAGAC
CCAGACCAACAGCCCCAGAAGAGCCAGAAGCGTGGCCAGCCAGAGCATCATCGCCT
ACACCATGAGCCTGGGCGCCGAGAACAGCGTGGCCTACAGCAACAACAGCATCGCC
ATCCCCACCAACTTCACCATCAGCGTGACCACCGAGATCCTGCCCGTGAGCATGACC
AAGACCAGCGTGGACTGCACCATGTACATCTGCGGCGACAGCACCGAGTGCAGCAA
CCTGCTGCTGCAGTACGGCAGCTTCTGCACCCAGCTGAACAGAGCCCTGACCGGCAT
CGCCGTGGAGCAGGACAAGAACACCCAGGAGGTGTTCGCCCAGGTGAAGCAGATCT
ACAAGACCCCCCCCATCAAGGACTTCGGCGGCTTCAACTTCAGCCAGATCCTGCCCG
ACCCCAGCAAGCCCAGCAAGAGAAGCTTCATCGAGGACCTGCTGTTCAACAAGGTG
ACCCTGGCCGACGCCGGCTTCATCAAGCAGTACGGCGACTGCCTGGGCGACATCGC
CGCCAGAGACCTGATCTGCGCCCAGAAGTTCAACGGCCTGACCGTGCTGCCCCCCCT
GCTGACCGACGAGATGATCGCCCAGTACACCAGCGCCCTGCTGGCCGGCACCATCA
CCAGCGGCTGGACCTTCGGCGCCGGCGCCGCCCTGCAGATCCCCTTCGCCATGCAGA
TGGCCTACAGATTCAACGGCATCGGCGTGACCCAGAACGTGCTGTACGAGAACCAG
AAGCTGATCGCCAACCAGTTCAACAGCGCCATCGGCAAGATCCAGGACAGCCTGAG
CAGCACCGCCAGCGCCCTGGGCAAGCTGCAGGACGTGGTGAACCAGAACGCCCAGG
CCCTGAACACCCTGGTGAAGCAGCTGAGCAGCAACTTCGGCGCCATCAGCAGCGTG
CTGAACGATATCCTGAGCAGACTGGACCCACCTGAGGCCGAGGTGCAGATCGACAG
ACTGATCACCGGCAGACTGCAGAGCCTGCAGACCTACGTGACCCAGCAGCTGATCA
GAGCCGCCGAGATCAGAGCCAGCGCCAACCTGGCCGCCACCAAGATGAGCGAGTGC
GTGCTGGGCCAGAGCAAGAGAGTGGACTTCTGCGGCAAGGGCTACCACCTGATGAG
CTTCCCCCAGAGCGCCCCCCACGGCGTGGTGTTCCTGCACGTGACCTACGTGCCCGC
CCAGGAGAAGAACTTCACCACCGCCCCCGCCATCTGCCACGACGGCAAGGCCCACT
TCCCCAGAGAGGGCGTGTTCGTGAGCAACGGCACCCACTGGTTCGTGACCCAGAGA
AACTTCTACGAGCCCCAGATCATCACCACCGACAACACCTTCGTGAGCGGCAACTGC
GACGTGGTGATCGGCATCGTGAACAACACCGTGTACGACCCCCTGCAGCCCGAGCT
GGACAGCTTCAAGGAGGAGCTGGACAAGTACTTCAAGAACCACACCAGCCCCGACG
TGGACCTGGGCGACATCAGCGGCATCAACGCCAGCGTGGTGAACATCCAGAAGGAG
ATCGACAGACTGAACGAGGTGGCCAAGAATTTAAATGAGAGCCTGATCGACCTGCA
GGAGCTGGGCAAGTACGAGCAGTACATCAAGTGGCCCTGGTACATCTGGCTGGGCT
TCATCGCCGGCCTGATCGCCATCGTGATGGTGACCATCATGCTGTGCTGCATGACCA
GCTGCTGCAGCTGCCTGAAGGGCTGCTGCAGCTGCGGCAGCTGCTGCAAGTTCGAC
GAGGACGACAGCGAGCCCGTGCTGAAGGGCGTGAAGCTGCACTACACCTAAACTAG
AAATCAACCTCTGGATTACAAAATTTGTGAAAGATTGACTGGTATTCTTAACTATGT
TGCTCCTTTTACGCTATGTGGATACGCTGCTTTAATGCCTTTGTATCATGCTATTGCTT
CCCGTATGGCTTTCATTTTCTCCTCCTTGTATAAATCCTGGTTGCTGTCTCTTTATGAG
GAGTTGTGGCCCGTTGTCAGGCAACGTGGCGTGGTGTGCACTGTGTTTGCTGACGCA
ACCCCCACTGGTTGGGGCATTGCCACCACCTGTCAGCTCCTTTCCGGGACTTTCGCTT
TCCCCCTCCCTATTGCCACGGCGGAACTCATCGCCGCCTGCCTTGCCCGCTGCTGGA
CAGGGGCTCGGCTGTTGGGCACTGACAATTCCGTGGTGTTGTCGGGGAAATCATCGT
CCTTTCCTTGGCTGCTCGCCTGTGTTGCCACCTGGATTCTGCGCGGGACGTCCTTCTG
CTACGTCCCTTCGGCCCTCAATCCAGCGGACCTTCCTTCCCGCGGCCTGCTGCCGGCT
CTGCGGCCTCTTCCGCGTCTTCGCCTTCGCCCTCAGACGAGTCGGATCTCCCTTTGGG
CCGCCTCCCCGCCAGACATGATAAGATACATTGATGAGTTTGGACAAACCACAACT
AGAATGCAGTGAAAAAAATGCTTTATTTGTGAAATTTGTGATGCTATTGCTTTATTTG
TAACCATTATAAGCTGCAATAAACAAGTTAACAACAACAATTGCATTCATTTTATGT
TTCAGGTTCAGGGGGAGGTGTGGGAGGTTTTTTAAAGCAAGTAAAACCTCTACAAAT
GTGGTA
SEQ ID NO: 8-Spike D614G-PP-IRES-hCD40L-WPRE-polyA
ATGTTCGTGTTCCTGGTGCTGCTGCCCCTGGTGAGCAGCCAGTGCGTGAACCTGACC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGACAACCCCGTGCTGCCCTTCAACGACGGCGTGTACTTCGCCAGCA
CCGAGAAGAGCAACATCATCAGAGGCTGGATCTTCGGCACCACCCTGGACAGCAAG
ACCCAGAGCCTGCTGATCGTGAACAACGCCACCAACGTGGTGATCAAGGTGTGCGA
GTTCCAGTTCTGCAACGACCCCTTCCTGGGCGTGTACTACCACAAGAACAACAAGAG
CTGGATGGAGAGCGAGTTCAGAGTGTACAGCAGCGCCAACAACTGCACCTTCGAGT
ACGTGAGCCAGCCCTTCCTGATGGACCTGGAGGGCAAGCAGGGCAACTTCAAGAAC
CTGAGAGAGTTCGTGTTCAAGAACATCGACGGCTACTTCAAGATCTACAGCAAGCA
CACCCCCATCAACCTGGTGAGAGACCTGCCCCAGGGCTTCAGCGCCCTGGAGCCCCT
GGTGGACCTGCCCATCGGCATCAACATCACCAGATTCCAGACCCTGCTGGCCCTGCA
CAGAAGCTACCTGACCCCCGGCGACAGCAGCAGCGGCTGGACCGCCGGCGCCGCCG
CCTACTACGTGGGCTACCTGCAGCCCAGAACCTTCCTGCTGAAGTACAACGAGAAC
GGCACCATCACCGACGCCGTGGACTGCGCCCTGGACCCCCTGAGCGAGACCAAGTG
CACCCTGAAGAGCTTCACCGTGGAGAAGGGCATCTACCAGACCAGCAACTTCAGAG
TGCAGCCCACCGAGAGCATCGTGAGATTCCCCAACATCACCAACCTGTGCCCCTTCG
GCGAGGTGTTCAACGCCACCAGATTCGCCAGCGTGTACGCCTGGAACAGAAAGAGA
ATCAGCAACTGCGTGGCCGACTACAGCGTGCTGTACAACAGCGCCAGCTTCAGCAC
CTTCAAGTGCTACGGCGTGAGCCCCACCAAGCTGAACGACCTGTGCTTCACCAACGT
GTACGCCGACAGCTTCGTGATCAGAGGCGACGAGGTGAGACAGATCGCCCCCGGCC
AGACCGGCAAGATCGCCGACTACAACTACAAGCTGCCCGACGACTTCACCGGCTGC
GTGATCGCCTGGAACAGCAACAACCTGGACAGCAAGGTGGGCGGCAACTACAACTA
CCTGTACAGACTGTTCAGAAAGAGCAACCTGAAGCCCTTCGAGAGAGACATCAGCA
CCGAGATCTACCAGGCCGGCAGCACCCCCTGCAACGGCGTGGAGGGCTTCAACTGC
TACTTCCCCCTGCAGAGCTACGGCTTCCAGCCCACCAACGGCGTGGGCTACCAGCCC
TACAGAGTGGTGGTGCTGAGCTTCGAGCTGCTGCACGCCCCCGCCACCGTGTGCGGC
CCCAAGAAGAGCACCAACCTGGTGAAGAACAAGTGCGTGAACTTCAACTTCAACGG
CCTGACCGGCACCGGCGTGCTGACCGAGAGCAACAAGAAGTTCCTGCCCTTCCAGC
AGTTCGGCAGAGACATCGCCGACACCACCGACGCCGTGAGAGACCCCCAGACCCTG
GAGATCCTGGACATCACCCCCTGCAGCTTCGGCGGCGTGAGCGTGATCACCCCCGGC
ACCAACACCAGCAACCAGGTGGCCGTGCTGTACCAGGGCGTGAACTGCACCGAGGT
GCCCGTGGCCATCCACGCCGACCAGCTGACCCCCACCTGGAGAGTGTACAGCACCG
GCAGCAACGTGTTCCAGACCAGAGCCGGCTGCCTGATCGGCGCCGAGCACGTGAAC
AACAGCTACGAGTGCGACATCCCCATCGGCGCCGGCATCTGCGCCAGCTACCAGAC
CCAGACCAACAGCCCCAGAAGAGCCAGAAGCGTGGCCAGCCAGAGCATCATCGCCT
ACACCATGAGCCTGGGCGCCGAGAACAGCGTGGCCTACAGCAACAACAGCATCGCC
ATCCCCACCAACTTCACCATCAGCGTGACCACCGAGATCCTGCCCGTGAGCATGACC
AAGACCAGCGTGGACTGCACCATGTACATCTGCGGCGACAGCACCGAGTGCAGCAA
CCTGCTGCTGCAGTACGGCAGCTTCTGCACCCAGCTGAACAGAGCCCTGACCGGCAT
CGCCGTGGAGCAGGACAAGAACACCCAGGAGGTGTTCGCCCAGGTGAAGCAGATCT
ACAAGACCCCCCCCATCAAGGACTTCGGCGGCTTCAACTTCAGCCAGATCCTGCCCG
ACCCCAGCAAGCCCAGCAAGAGAAGCTTCATCGAGGACCTGCTGTTCAACAAGGTG
ACCCTGGCCGACGCCGGCTTCATCAAGCAGTACGGCGACTGCCTGGGCGACATCGC
CGCCAGAGACCTGATCTGCGCCCAGAAGTTCAACGGCCTGACCGTGCTGCCCCCCCT
GCTGACCGACGAGATGATCGCCCAGTACACCAGCGCCCTGCTGGCCGGCACCATCA
CCAGCGGCTGGACCTTCGGCGCCGGCGCCGCCCTGCAGATCCCCTTCGCCATGCAGA
TGGCCTACAGATTCAACGGCATCGGCGTGACCCAGAACGTGCTGTACGAGAACCAG
AAGCTGATCGCCAACCAGTTCAACAGCGCCATCGGCAAGATCCAGGACAGCCTGAG
CAGCACCGCCAGCGCCCTGGGCAAGCTGCAGGACGTGGTGAACCAGAACGCCCAGG
CCCTGAACACCCTGGTGAAGCAGCTGAGCAGCAACTTCGGCGCCATCAGCAGCGTG
CTGAACGATATCCTGAGCAGACTGGACCCACCTGAGGCCGAGGTGCAGATCGACAG
ACTGATCACCGGCAGACTGCAGAGCCTGCAGACCTACGTGACCCAGCAGCTGATCA
GAGCCGCCGAGATCAGAGCCAGCGCCAACCTGGCCGCCACCAAGATGAGCGAGTGC
GTGCTGGGCCAGAGCAAGAGAGTGGACTTCTGCGGCAAGGGCTACCACCTGATGAG
CTTCCCCCAGAGCGCCCCCCACGGCGTGGTGTTCCTGCACGTGACCTACGTGCCCGC
CCAGGAGAAGAACTTCACCACCGCCCCCGCCATCTGCCACGACGGCAAGGCCCACT
TCCCCAGAGAGGGCGTGTTCGTGAGCAACGGCACCCACTGGTTCGTGACCCAGAGA
AACTTCTACGAGCCCCAGATCATCACCACCGACAACACCTTCGTGAGCGGCAACTGC
GACGTGGTGATCGGCATCGTGAACAACACCGTGTACGACCCCCTGCAGCCCGAGCT
GGACAGCTTCAAGGAGGAGCTGGACAAGTACTTCAAGAACCACACCAGCCCCGACG
TGGACCTGGGCGACATCAGCGGCATCAACGCCAGCGTGGTGAACATCCAGAAGGAG
ATCGACAGACTGAACGAGGTGGCCAAGAATTTAAATGAGAGCCTGATCGACCTGCA
GGAGCTGGGCAAGTACGAGCAGTACATCAAGTGGCCCTGGTACATCTGGCTGGGCT
TCATCGCCGGCCTGATCGCCATCGTGATGGTGACCATCATGCTGTGCTGCATGACCA
GCTGCTGCAGCTGCCTGAAGGGCTGCTGCAGCTGCGGCAGCTGCTGCAAGTTCGAC
GAGGACGACAGCGAGCCCGTGCTGAAGGGCGTGAAGCTGCACTACACCTAAACTAG
TCGCCCCTCTCCCTCCCCCCCCCCTAACGTTACTGGCCGAAGCCGCTTGGAATAAGG
CCGGTGTGCGTTTGTCTATATGTTATTTTCCACCATATTGCCGTCTTTTGGCAATGTG
AGGGCCCGGAAACCTGGCCCTGTCTTCTTGACGAGCATTCCTAGGGGTCTTTCCCCT
CTCGCCAAAGGAATGCAAGGTCTGTTGAATGTCGTGAAGGAAGCAGTTCCTCTGGA
AGCTTCTTGAAGACAAACAACGTCTGTAGCGACCCTTTGCAGGCAGCGGAACCCCC
CACCTGGCGACAGGTGCCTCTGCGGCCAAAAGCCACGTGTATAAGATACACCTGCA
AAGGCGGCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGGTACCCC
ATTGTATGGGATCTGATCTGGGGCCTCGGTGCACATGCTTTACATGTGTTTAGTCGA
GGTTAAAAAACGTCTAGGCCCCCCGAACCACGGGGACGTGGTTTTCCTTTGAAAAAC
ACGATGATAATATGATCGAAACATACAACCAAACTTCTCCCCGATCTGCGGCCACTG
GACTGCCCATCAGCATGAAAATTTTTATGTATCTCCTTACTGTTTTTCTTATCACCCA
GATGATTGGGTCAGCACTTTTTGCTGTGTATCTTCATAGAAGGTTGGACAAGATAGA
AGATGAAAGGAATCTTCATGAAGATTTTGTATTCATGAAAACGATACAGAGATGCA
ACACAGGAGAAAGATCCTTATCCTTACTGAACTGTGAGGAGATCAAAAGCCAGTTT
GAAGGCTTTGTGAAGGATATAATGTTAAACAAAGAGGAGACGAAGAAAGAAAACA
GCGGAGATCAGAATCCTCAAATTGCGGCACATGTCATAAGTGAGGCCAGCAGTAAA
ACAACATCTGTGTTACAGTGGGCTGAAAAAGGATACTACACCATGAGCAACAACTT
GGTAACCCTGGAAAATGGGAAACAGCTGACCGTTAAAAGACAAGGACTCTATTATA
TCTATGCCCAAGTCACCTTCTGTTCCAATCGGGAAGCTTCGAGTCAAGCTCCCTTTAT
AGCCAGCCTCTGCCTAAAGTCCCCCGGTAGATTCGAGAGAATCTTACTCAGAGCTGC
AAATACCCACAGTTCCGCCAAACCTTGCGGGCAACAATCCATTCACTTGGGAGGAG
TATTTGAATTGCAACCAGGTGCTTCGGTGTTTGTCAATGTGACTGATCCAAGCCAAG
TGAGCCATGGCACTGGCTTCACGTCCTTTGGCTTACTCAAACTCTGATCTAGAAATC
AACCTCTGGATTACAAAATTTGTGAAAGATTGACTGGTATTCTTAACTATGTTGCTCC
TTTTACGCTATGTGGATACGCTGCTTTAATGCCTTTGTATCATGCTATTGCTTCCCGT
ATGGCTTTCATTTTCTCCTCCTTGTATAAATCCTGGTTGCTGTCTCTTTATGAGGAGTT
GTGGCCCGTTGTCAGGCAACGTGGCGTGGTGTGCACTGTGTTTGCTGACGCAACCCC
CACTGGTTGGGGCATTGCCACCACCTGTCAGCTCCTTTCCGGGACTTTCGCTTTCCCC
CTCCCTATTGCCACGGCGGAACTCATCGCCGCCTGCCTTGCCCGCTGCTGGACAGGG
GCTCGGCTGTTGGGCACTGACAATTCCGTGGTGTTGTCGGGGAAATCATCGTCCTTT
CCTTGGCTGCTCGCCTGTGTTGCCACCTGGATTCTGCGCGGGACGTCCTTCTGCTACG
TCCCTTCGGCCCTCAATCCAGCGGACCTTCCTTCCCGCGGCCTGCTGCCGGCTCTGCG
GCCTCTTCCGCGTCTTCGCCTTCGCCCTCAGACGAGTCGGATCTCCCTTTGGGCCGCC
TCCCCGCCAGACATGATAAGATACATTGATGAGTTTGGACAAACCACAACTAGAAT
GCAGTGAAAAAAATGCTTTATTTGTGAAATTTGTGATGCTATTGCTTTATTTGTAACC
ATTATAAGCTGCAATAAACAAGTTAACAACAACAATTGCATTCATTTTATGTTTCAG
GTTCAGGGGGAGGTGTGGGAGGTTTTTTAAAGCAAGTAAAACCTCTACAAATGTGG
TA
SEQ ID NO: 9-Spike S1-Foldon-WPRE-polyA
ATGTTCGTGTTCCTGGTGCTGCTGCCCCTGGTGAGCAGCCAGTGCGTGAACCTGACC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGACAACCCCGTGCTGCCCTTCAACGACGGCGTGTACTTCGCCAGCA
CCGAGAAGAGCAACATCATCAGAGGCTGGATTTTCGGCACCACCCTGGACAGCAAG
ACCCAGAGCCTGCTGATCGTGAACAACGCCACCAACGTGGTGATCAAGGTGTGCGA
GTTCCAGTTCTGCAACGACCCCTTCCTGGGCGTGTACTACCACAAGAACAACAAGAG
CTGGATGGAGAGCGAGTTCAGAGTGTACAGCAGCGCCAACAACTGCACCTTCGAGT
ACGTGAGCCAGCCCTTCCTGATGGACCTGGAGGGCAAGCAGGGCAACTTCAAGAAC
CTGAGAGAGTTCGTGTTCAAGAACATCGACGGCTACTTCAAGATTTACAGCAAGCAC
ACCCCCATCAACCTGGTGAGAGACCTGCCCCAGGGCTTCAGCGCCCTGGAGCCCCTG
GTGGACCTGCCCATCGGCATCAACATCACCAGATTCCAGACCCTGCTGGCCCTGCAC
AGAAGCTACCTGACCCCCGGCGACAGCAGCAGCGGCTGGACCGCCGGCGCCGCCGC
CTACTACGTGGGCTACCTGCAGCCCAGAACCTTCCTGCTGAAGTACAACGAGAACG
GCACCATCACCGACGCCGTGGACTGCGCCCTGGACCCCCTGAGCGAGACCAAGTGC
ACCCTGAAGAGCTTCACCGTGGAGAAGGGCATCTACCAGACCAGCAACTTCAGAGT
GCAGCCCACCGAGAGCATCGTGAGATTCCCCAACATCACCAACCTGTGCCCCTTCGG
CGAGGTGTTCAACGCCACCAGATTCGCCAGCGTGTACGCCTGGAACAGAAAGAGAA
TCAGCAACTGCGTGGCCGACTACAGCGTGCTGTACAACAGCGCCAGCTTCAGCACCT
TCAAGTGCTACGGCGTGAGCCCCACCAAGCTGAACGACCTGTGCTTCACCAACGTGT
ACGCCGACAGCTTCGTGATCAGAGGCGACGAGGTGAGACAGATCGCCCCCGGCCAG
ACCGGCAAGATCGCCGACTACAACTACAAGCTGCCCGACGACTTCACCGGCTGCGT
GATCGCCTGGAACAGCAACAACCTGGACAGCAAGGTGGGCGGCAACTACAACTACC
TGTACAGACTGTTCAGAAAGAGCAACCTGAAGCCCTTCGAGAGAGACATCAGCACC
GAGATTTACCAGGCCGGCAGCACCCCCTGCAACGGCGTGGAGGGCTTCAACTGCTA
CTTCCCCCTGCAGAGCTACGGCTTCCAGCCCACCAACGGCGTGGGCTACCAGCCCTA
CAGAGTGGTGGTGCTGAGCTTCGAGCTGCTGCACGCCCCCGCCACCGTGTGCGGCCC
CAAGAAGAGCACCAACCTGGTGAAGAACAAGTGCGTGAACTTCAACTTCAACGGCC
TGACCGGCACCGGCGTGCTGACCGAGAGCAACAAGAAGTTCCTGCCCTTCCAGCAG
TTCGGCAGAGACATCGCCGACACCACCGACGCCGTGAGAGACCCCCAGACCCTGGA
GATTCTGGACATCACCCCCTGCAGCTTCGGCGGCGTGAGCGTGATCACCCCCGGCAC
CAACACCAGCAACCAGGTGGCCGTGCTGTACCAGGGCGTGAACTGCACCGAGGTGC
CCGTGGCCATCCACGCCGACCAGCTGACCCCCACCTGGAGAGTGTACAGCACCGGC
AGCAACGTGTTCCAGACCAGAGCCGGCTGCCTGATCGGCGCCGAGCACGTGAACAA
CAGCTACGAGTGCGACATCCCCATCGGCGCCGGCATCTGCGCCAGCTACCAGACCC
AGACCAACAGCGGCAGCGGCTACATCCCCGAGGCCCCCAGAGACGGCCAGGCCTAC
GTGAGAAAGGACGGCGAGTGGGTGCTGCTGAGCACCTTCCTGGGCTAAACTAGAAA
TCAACCTCTGGATTACAAAATTTGTGAAAGATTGACTGGTATTCTTAACTATGTTGCT
CCTTTTACGCTATGTGGATACGCTGCTTTAATGCCTTTGTATCATGCTATTGCTTCCC
GTATGGCTTTCATTTTCTCCTCCTTGTATAAATCCTGGTTGCTGTCTCTTTATGAGGA
GTTGTGGCCCGTTGTCAGGCAACGTGGCGTGGTGTGCACTGTGTTTGCTGACGCAAC
CCCCACTGGTTGGGGCATTGCCACCACCTGTCAGCTCCTTTCCGGGACTTTCGCTTTC
CCCCTCCCTATTGCCACGGCGGAACTCATCGCCGCCTGCCTTGCCCGCTGCTGGACA
GGGGCTCGGCTGTTGGGCACTGACAATTCCGTGGTGTTGTCGGGGAAATCATCGTCC
TTTCCTTGGCTGCTCGCCTGTGTTGCCACCTGGATTCTGCGCGGGACGTCCTTCTGCT
ACGTCCCTTCGGCCCTCAATCCAGCGGACCTTCCTTCCCGCGGCCTGCTGCCGGCTCT
GCGGCCTCTTCCGCGTCTTCGCCTTCGCCCTCAGACGAGTCGGATCTCCCTTTGGGCC
GCCTCCCCGCCAGACATGATAAGATACATTGATGAGTTTGGACAAACCACAACTAG
AATGCAGTGAAAAAAATGCTTTATTTGTGAAATTTGTGATGCTATTGCTTTATTTGTA
ACCATTATAAGCTGCAATAAACAAGTTAACAACAACAATTGCATTCATTTTATGTTT
CAGGTTCAGGGGGAGGTGTGGGAGGTTTTTTAAAGCAAGTAAAACCTCTACAAATG
TGGTA
SEQ ID NO: 10-Spike S1-Foldon-IRES-hCD40L-WPRE-polyA
ATGTTCGTGTTCCTGGTGCTGCTGCCCCTGGTGAGCAGCCAGTGCGTGAACCTGACC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGACAACCCCGTGCTGCCCTTCAACGACGGCGTGTACTTCGCCAGCA
CCGAGAAGAGCAACATCATCAGAGGCTGGATTTTCGGCACCACCCTGGACAGCAAG
ACCCAGAGCCTGCTGATCGTGAACAACGCCACCAACGTGGTGATCAAGGTGTGCGA
GTTCCAGTTCTGCAACGACCCCTTCCTGGGCGTGTACTACCACAAGAACAACAAGAG
CTGGATGGAGAGCGAGTTCAGAGTGTACAGCAGCGCCAACAACTGCACCTTCGAGT
ACGTGAGCCAGCCCTTCCTGATGGACCTGGAGGGCAAGCAGGGCAACTTCAAGAAC
CTGAGAGAGTTCGTGTTCAAGAACATCGACGGCTACTTCAAGATTTACAGCAAGCAC
ACCCCCATCAACCTGGTGAGAGACCTGCCCCAGGGCTTCAGCGCCCTGGAGCCCCTG
GTGGACCTGCCCATCGGCATCAACATCACCAGATTCCAGACCCTGCTGGCCCTGCAC
AGAAGCTACCTGACCCCCGGCGACAGCAGCAGCGGCTGGACCGCCGGCGCCGCCGC
CTACTACGTGGGCTACCTGCAGCCCAGAACCTTCCTGCTGAAGTACAACGAGAACG
GCACCATCACCGACGCCGTGGACTGCGCCCTGGACCCCCTGAGCGAGACCAAGTGC
ACCCTGAAGAGCTTCACCGTGGAGAAGGGCATCTACCAGACCAGCAACTTCAGAGT
GCAGCCCACCGAGAGCATCGTGAGATTCCCCAACATCACCAACCTGTGCCCCTTCGG
CGAGGTGTTCAACGCCACCAGATTCGCCAGCGTGTACGCCTGGAACAGAAAGAGAA
TCAGCAACTGCGTGGCCGACTACAGCGTGCTGTACAACAGCGCCAGCTTCAGCACCT
TCAAGTGCTACGGCGTGAGCCCCACCAAGCTGAACGACCTGTGCTTCACCAACGTGT
ACGCCGACAGCTTCGTGATCAGAGGCGACGAGGTGAGACAGATCGCCCCCGGCCAG
ACCGGCAAGATCGCCGACTACAACTACAAGCTGCCCGACGACTTCACCGGCTGCGT
GATCGCCTGGAACAGCAACAACCTGGACAGCAAGGTGGGCGGCAACTACAACTACC
TGTACAGACTGTTCAGAAAGAGCAACCTGAAGCCCTTCGAGAGAGACATCAGCACC
GAGATTTACCAGGCCGGCAGCACCCCCTGCAACGGCGTGGAGGGCTTCAACTGCTA
CTTCCCCCTGCAGAGCTACGGCTTCCAGCCCACCAACGGCGTGGGCTACCAGCCCTA
CAGAGTGGTGGTGCTGAGCTTCGAGCTGCTGCACGCCCCCGCCACCGTGTGCGGCCC
CAAGAAGAGCACCAACCTGGTGAAGAACAAGTGCGTGAACTTCAACTTCAACGGCC
TGACCGGCACCGGCGTGCTGACCGAGAGCAACAAGAAGTTCCTGCCCTTCCAGCAG
TTCGGCAGAGACATCGCCGACACCACCGACGCCGTGAGAGACCCCCAGACCCTGGA
GATTCTGGACATCACCCCCTGCAGCTTCGGCGGCGTGAGCGTGATCACCCCCGGCAC
CAACACCAGCAACCAGGTGGCCGTGCTGTACCAGGGCGTGAACTGCACCGAGGTGC
CCGTGGCCATCCACGCCGACCAGCTGACCCCCACCTGGAGAGTGTACAGCACCGGC
AGCAACGTGTTCCAGACCAGAGCCGGCTGCCTGATCGGCGCCGAGCACGTGAACAA
CAGCTACGAGTGCGACATCCCCATCGGCGCCGGCATCTGCGCCAGCTACCAGACCC
AGACCAACAGCGGCAGCGGCTACATCCCCGAGGCCCCCAGAGACGGCCAGGCCTAC
GTGAGAAAGGACGGCGAGTGGGTGCTGCTGAGCACCTTCCTGGGCTAAACTAGTCG
CCCCTCTCCCTCCCCCCCCCCTAACGTTACTGGCCGAAGCCGCTTGGAATAAGGCCG
GTGTGCGTTTGTCTATATGTTATTTTCCACCATATTGCCGTCTTTTGGCAATGTGAGG
GCCCGGAAACCTGGCCCTGTCTTCTTGACGAGCATTCCTAGGGGTCTTTCCCCTCTCG
CCAAAGGAATGCAAGGTCTGTTGAATGTCGTGAAGGAAGCAGTTCCTCTGGAAGCT
TCTTGAAGACAAACAACGTCTGTAGCGACCCTTTGCAGGCAGCGGAACCCCCCACCT
GGCGACAGGTGCCTCTGCGGCCAAAAGCCACGTGTATAAGATACACCTGCAAAGGC
GGCACAACCCCAGTGCCACGTTGTGAGTTGGATAGTTGTGGAAAGAGTCAAATGGC
TCTCCTCAAGCGTATTCAACAAGGGGCTGAAGGATGCCCAGAAGGTACCCCATTGTA
TGGGATCTGATCTGGGGCCTCGGTGCACATGCTTTACATGTGTTTAGTCGAGGTTAA
AAAACGTCTAGGCCCCCCGAACCACGGGGACGTGGTTTTCCTTTGAAAAACACGAT
GATAATATGATCGAAACATACAACCAAACTTCTCCCCGATCTGCGGCCACTGGACTG
CCCATCAGCATGAAAATTTTTATGTATCTCCTTACTGTTTTTCTTATCACCCAGATGA
TTGGGTCAGCACTTTTTGCTGTGTATCTTCATAGAAGGTTGGACAAGATAGAAGATG
AAAGGAATCTTCATGAAGATTTTGTATTCATGAAAACGATACAGAGATGCAACACA
GGAGAAAGATCCTTATCCTTACTGAACTGTGAGGAGATCAAAAGCCAGTTTGAAGG
CTTTGTGAAGGATATAATGTTAAACAAAGAGGAGACGAAGAAAGAAAACAGCGGA
GATCAGAATCCTCAAATTGCGGCACATGTCATAAGTGAGGCCAGCAGTAAAACAAC
ATCTGTGTTACAGTGGGCTGAAAAAGGATACTACACCATGAGCAACAACTTGGTAA
CCCTGGAAAATGGGAAACAGCTGACCGTTAAAAGACAAGGACTCTATTATATCTAT
GCCCAAGTCACCTTCTGTTCCAATCGGGAAGCTTCGAGTCAAGCTCCCTTTATAGCC
AGCCTCTGCCTAAAGTCCCCCGGTAGATTCGAGAGAATCTTACTCAGAGCTGCAAAT
ACCCACAGTTCCGCCAAACCTTGCGGGCAACAATCCATTCACTTGGGAGGAGTATTT
GAATTGCAACCAGGTGCTTCGGTGTTTGTCAATGTGACTGATCCAAGCCAAGTGAGC
CATGGCACTGGCTTCACGTCCTTTGGCTTACTCAAACTCTGATCTAGAAATCAACCTC
TGGATTACAAAATTTGTGAAAGATTGACTGGTATTCTTAACTATGTTGCTCCTTTTAC
GCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCC
CGTTGTCAGGCAACGTGGCGTGGTGTGCACTGTGTTTGCTGACGCAACCCCCACTGG
TTGGGGCATTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGGGGCTCGG
CTGTTGGGCACTGACAATTCCGTGGTGTTGTCGGGGAAATCATCGTCCTTTCCTTGGC
TGCTCGCCTGTGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTGCGGCCTCT
TCCGCGTCTTCGCCTTCGCCCTCAGACGAGTCGGATCTCCCTTTGGGCCGCCTCCCCG
CCAGACATGATAAGATACATTGATGAGTTTGGACAAACCACAACTAGAATGCAGTG
AAAAAAATGCTTTATTTGTGAAATTTGTGATGCTATTGCTTTATTTGTAACCATTATA
AGCTGCAATAAACAAGTTAACAACAACAATTGCATTCATTTTATGTTTCAGGTTCAG
GGGGAGGTGTGGGAGGTTTTTTAAAGCAAGTAAAACCTCTACAAATGTGGTA
SEQ ID NO: 11
CRGDC
SEQ ID NO: 12
CXCRGDCXC (X represents any amino acid)
SEQ ID NO: 13
CDCRGDCFC
SEQ ID NO: 14-SARS-CoV-2 spike protein, deposited with the NCBI under
Accession No. QHD43416
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVENATRFASVYAWNRKRISN
CVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGST
PCNGVEGENCYFPLQSYGFQPTNGVGYQPYRVVVLSFELLHAPATVCGPKKSTNLVKN
KCVNFNFNGLTGTGVLTESNKKFLPFQQFGRDIADTTDAVRDPQTLEILDITPCSFGGVS
VITPGTNTSNQVAVLYQDVNCTEVPVAIHADQLTPTWRVYSTGSNVFQTRAGCLIGAEH
VNNSYECDIPIGAGICASYQTQTNSPRRARSVASQSIIAYTMSLGAENSVAYSNNSIAIPT
NFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGWTFGAGAALQIP
FAMQMAYRFNGIGVTQNVLYENQKLIANQFNSAIGKIQDSLSSTASALGKLQDVVNQN
AQALNTLVKQLSSNFGAISSVLNDILSRLDKVEAEVQIDRLITGRLQSLQTYVTQQLIRAA
EIRASANLAATKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNLN
ESLIDLQELGKYEQYIKWPWYIWLGFIAGLIAIVMVTIMLCCMTSCCSCLKGCCSCGSCC
KFDEDDSEPVLKGVKLHYT
SEQ ID NO: 15-CoroVaxG.3 spike protein-nucleic acid sequence
ATGTTCGTGTTCCTGGTGCTGCTGCCCCTGGTGAGCAGCCAGTGCGTGAACCTGACC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGACAACCCCGTGCTGCCCTTCAACGACGGCGTGTACTTCGCCAGCA
CCGAGAAGAGCAACATCATCAGAGGCTGGATCTTCGGCACCACCCTGGACAGCAAG
ACCCAGAGCCTGCTGATCGTGAACAACGCCACCAACGTGGTGATCAAGGTGTGCGA
GTTCCAGTTCTGCAACGACCCCTTCCTGGGCGTGTACTACCACAAGAACAACAAGAG
CTGGATGGAGAGCGAGTTCAGAGTGTACAGCAGCGCCAACAACTGCACCTTCGAGT
ACGTGAGCCAGCCCTTCCTGATGGACCTGGAGGGCAAGCAGGGCAACTTCAAGAAC
CTGAGAGAGTTCGTGTTCAAGAACATCGACGGCTACTTCAAGATCTACAGCAAGCA
CACCCCCATCAACCTGGTGAGAGACCTGCCCCAGGGCTTCAGCGCCCTGGAGCCCCT
GGTGGACCTGCCCATCGGCATCAACATCACCAGATTCCAGACCCTGCTGGCCCTGCA
CAGAAGCTACCTGACCCCCGGCGACAGCAGCAGCGGCTGGACCGCCGGCGCCGCCG
CCTACTACGTGGGCTACCTGCAGCCCAGAACCTTCCTGCTGAAGTACAACGAGAAC
GGCACCATCACCGACGCCGTGGACTGCGCCCTGGACCCCCTGAGCGAGACCAAGTG
CACCCTGAAGAGCTTCACCGTGGAGAAGGGCATCTACCAGACCAGCAACTTCAGAG
TGCAGCCCACCGAGAGCATCGTGAGATTCCCCAACATCACCAACCTGTGCCCCTTCG
GCGAGGTGTTCAACGCCACCAGATTCGCCAGCGTGTACGCCTGGAACAGAAAGAGA
ATCAGCAACTGCGTGGCCGACTACAGCGTGCTGTACAACAGCGCCAGCTTCAGCAC
CTTCAAGTGCTACGGCGTGAGCCCCACCAAGCTGAACGACCTGTGCTTCACCAACGT
GTACGCCGACAGCTTCGTGATCAGAGGCGACGAGGTGAGACAGATCGCCCCCGGCC
AGACCGGCAAGATCGCCGACTACAACTACAAGCTGCCCGACGACTTCACCGGCTGC
GTGATCGCCTGGAACAGCAACAACCTGGACAGCAAGGTGGGCGGCAACTACAACTA
CCTGTACAGACTGTTCAGAAAGAGCAACCTGAAGCCCTTCGAGAGAGACATCAGCA
CCGAGATCTACCAGGCCGGCAGCACCCCCTGCAACGGCGTGGAGGGCTTCAACTGC
TACTTCCCCCTGCAGAGCTACGGCTTCCAGCCCACCAACGGCGTGGGCTACCAGCCC
TACAGAGTGGTGGTGCTGAGCTTCGAGCTGCTGCACGCCCCCGCCACCGTGTGCGGC
CCCAAGAAGAGCACCAACCTGGTGAAGAACAAGTGCGTGAACTTCAACTTCAACGG
CCTGACCGGCACCGGCGTGCTGACCGAGAGCAACAAGAAGTTCCTGCCCTTCCAGC
AGTTCGGCAGAGACATCGCCGACACCACCGACGCCGTGAGAGACCCCCAGACCCTG
GAGATCCTGGACATCACCCCCTGCAGCTTCGGCGGCGTGAGCGTGATCACCCCCGGC
ACCAACACCAGCAACCAGGTGGCCGTGCTGTACCAGGGCGTGAACTGCACCGAGGT
GCCCGTGGCCATCCACGCCGACCAGCTGACCCCCACCTGGAGAGTGTACAGCACCG
GCAGCAACGTGTTCCAGACCAGAGCCGGCTGCCTGATCGGCGCCGAGCACGTGAAC
AACAGCTACGAGTGCGACATCCCCATCGGCGCCGGCATCTGCGCCAGCTACCAGAC
CCAGACCAACAGCCCCAGAAGAGCCAGAAGCGTGGCCAGCCAGAGCATCATCGCCT
ACACCATGAGCCTGGGCGCCGAGAACAGCGTGGCCTACAGCAACAACAGCATCGCC
ATCCCCACCAACTTCACCATCAGCGTGACCACCGAGATCCTGCCCGTGAGCATGACC
AAGACCAGCGTGGACTGCACCATGTACATCTGCGGCGACAGCACCGAGTGCAGCAA
CCTGCTGCTGCAGTACGGCAGCTTCTGCACCCAGCTGAACAGAGCCCTGACCGGCAT
CGCCGTGGAGCAGGACAAGAACACCCAGGAGGTGTTCGCCCAGGTGAAGCAGATCT
ACAAGACCCCCCCCATCAAGGACTTCGGCGGCTTCAACTTCAGCCAGATCCTGCCCG
ACCCCAGCAAGCCCAGCAAGAGAAGCTTCATCGAGGACCTGCTGTTCAACAAGGTG
ACCCTGGCCGACGCCGGCTTCATCAAGCAGTACGGCGACTGCCTGGGCGACATCGC
CGCCAGAGACCTGATCTGCGCCCAGAAGTTCAACGGCCTGACCGTGCTGCCCCCCCT
GCTGACCGACGAGATGATCGCCCAGTACACCAGCGCCCTGCTGGCCGGCACCATCA
CCAGCGGCTGGACCTTCGGCGCCGGCGCCGCCCTGCAGATCCCCTTCGCCATGCAGA
TGGCCTACAGATTCAACGGCATCGGCGTGACCCAGAACGTGCTGTACGAGAACCAG
AAGCTGATCGCCAACCAGTTCAACAGCGCCATCGGCAAGATCCAGGACAGCCTGAG
CAGCACCGCCAGCGCCCTGGGCAAGCTGCAGGACGTGGTGAACCAGAACGCCCAGG
CCCTGAACACCCTGGTGAAGCAGCTGAGCAGCAACTTCGGCGCCATCAGCAGCGTG
CTGAACGATATCCTGAGCAGACTGGACCCACCTGAGGCCGAGGTGCAGATCGACAG
ACTGATCACCGGCAGACTGCAGAGCCTGCAGACCTACGTGACCCAGCAGCTGATCA
GAGCCGCCGAGATCAGAGCCAGCGCCAACCTGGCCGCCACCAAGATGAGCGAGTGC
GTGCTGGGCCAGAGCAAGAGAGTGGACTTCTGCGGCAAGGGCTACCACCTGATGAG
CTTCCCCCAGAGCGCCCCCCACGGCGTGGTGTTCCTGCACGTGACCTACGTGCCCGC
CCAGGAGAAGAACTTCACCACCGCCCCCGCCATCTGCCACGACGGCAAGGCCCACT
TCCCCAGAGAGGGCGTGTTCGTGAGCAACGGCACCCACTGGTTCGTGACCCAGAGA
AACTTCTACGAGCCCCAGATCATCACCACCGACAACACCTTCGTGAGCGGCAACTGC
GACGTGGTGATCGGCATCGTGAACAACACCGTGTACGACCCCCTGCAGCCCGAGCT
GGACAGCTTCAAGGAGGAGCTGGACAAGTACTTCAAGAACCACACCAGCCCCGACG
TGGACCTGGGCGACATCAGCGGCATCAACGCCAGCGTGGTGAACATCCAGAAGGAG
ATCGACAGACTGAACGAGGTGGCCAAGAATTTAAATGAGAGCCTGATCGACCTGCA
GGAGCTGGGCAAGTACGAGCAGTACATCAAGTGGCCCTGGTACATCTGGCTGGGCT
TCATCGCCGGCCTGATCGCCATCGTGATGGTGACCATCATGCTGTGCTGCATGACCA
GCTGCTGCAGCTGCCTGAAGGGCTGCTGCAGCTGCGGCAGCTGCTGCAAGTTCGAC
GAGGACGACAGCGAGCCCGTGCTGAAGGGCGTGAAGCTGCACTACACCTAA
SEQ ID NO: 16-Coro VaxG.3 spike protein-amino acid sequence
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVENATRFASVYAWNRKRISN
CVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGST
PCNGVEGENCYFPLQSYGFQPTNGVGYQPYRVVVLSFELLHAPATVCGPKKSTNLVKN
KCVNFNFNGLTGTGVLTESNKKFLPFQQFGRDIADTTDAVRDPQTLEILDITPCSFGGVS
VITPGTNTSNQVAVLYQGVNCTEVPVAIHADQLTPTWRVYSTGSNVFQTRAGCLIGAEH
VNNSYECDIPIGAGICASYQTQTNSPRRARSVASQSIIAYTMSLGAENSVAYSNNSIAIPT
NFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGWTFGAGAALQIP
FAMQMAYRFNGIGVTQNVLYENQKLIANQFNSAIGKIQDSLSSTASALGKLQDVVNQN
AQALNTLVKQLSSNFGAISSVLNDILSRLDPPEAEVQIDRLITGRLQSLQTYVTQQLIRAA
EIRASANLAATKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNLN
ESLIDLQELGKYEQYIKWPWYIWLGFIAGLIAIVMVTIMLCCMTSCCSCLKGCCSCGSCC
KFDEDDSEPVLKGVKLHYT
SEQ ID NO: 17
ATGTTCGTGTTCCTGGTGCTGCTGCCCCTGGTGAGCAGCCAGTGCGTGAACCTGACC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCAGCGGCACCAACGGCACCAAGA
GATTCGACAACCCCGTGCTGCCCTTCAACGACGGCGTGTACTTCGCCAGCACCGAGA
AGAGCAACATCATCAGAGGCTGGATCTTCGGCACCACCCTGGACAGCAAGACCCAG
AGCCTGCTGATCGTGAACAACGCCACCAACGTGGTGATCAAGGTGTGCGAGTTCCA
GTTCTGCAACGACCCCTTCCTGGGCGTGTACCACAAGAACAACAAGAGCTGGATGG
AGAGCGAGTTCAGAGTGTACAGCAGCGCCAACAACTGCACCTTCGAGTACGTGAGC
CAGCCCTTCCTGATGGACCTGGAGGGCAAGCAGGGCAACTTCAAGAACCTGAGAGA
GTTCGTGTTCAAGAACATCGACGGCTACTTCAAGATCTACAGCAAGCACACCCCCAT
CAACCTGGTGAGAGACCTGCCCCAGGGCTTCAGCGCCCTGGAGCCCCTGGTGGACC
TGCCCATCGGCATCAACATCACCAGATTCCAGACCCTGCTGGCCCTGCACAGAAGCT
ACCTGACCCCCGGCGACAGCAGCAGCGGCTGGACCGCCGGCGCCGCCGCCTACTAC
GTGGGCTACCTGCAGCCCAGAACCTTCCTGCTGAAGTACAACGAGAACGGCACCAT
CACCGACGCCGTGGACTGCGCCCTGGACCCCCTGAGCGAGACCAAGTGCACCCTGA
AGAGCTTCACCGTGGAGAAGGGCATCTACCAGACCAGCAACTTCAGAGTGCAGCCC
ACCGAGAGCATCGTGAGATTCCCCAACATCACCAACCTGTGCCCCTTCGGCGAGGTG
TTCAACGCCACCAGATTCGCCAGCGTGTACGCCTGGAACAGAAAGAGAATCAGCAA
CTGCGTGGCCGACTACAGCGTGCTGTACAACAGCGCCAGCTTCAGCACCTTCAAGTG
CTACGGCGTGAGCCCCACCAAGCTGAACGACCTGTGCTTCACCAACGTGTACGCCG
ACAGCTTCGTGATCAGAGGCGACGAGGTGAGACAGATCGCCCCCGGCCAGACCGGC
AAGATCGCCGACTACAACTACAAGCTGCCCGACGACTTCACCGGCTGCGTGATCGC
CTGGAACAGCAACAACCTGGACAGCAAGGTGGGCGGCAACTACAACTACCTGTACA
GACTGTTCAGAAAGAGCAACCTGAAGCCCTTCGAGAGAGACATCAGCACCGAGATC
TACCAGGCCGGCAGCACCCCCTGCAACGGCGTGGAGGGCTTCAACTGCTACTTCCCC
CTGCAGAGCTACGGCTTCCAGCCCACCTACGGCGTGGGCTACCAGCCCTACAGAGT
GGTGGTGCTGAGCTTCGAGCTGCTGCACGCCCCCGCCACCGTGTGCGGCCCCAAGA
AGAGCACCAACCTGGTGAAGAACAAGTGCGTGAACTTCAACTTCAACGGCCTGACC
GGCACCGGCGTGCTGACCGAGAGCAACAAGAAGTTCCTGCCCTTCCAGCAGTTCGG
CAGAGACATCGACGACACCACCGACGCCGTGAGAGACCCCCAGACCCTGGAGATCC
TGGACATCACCCCCTGCAGCTTCGGCGGCGTGAGCGTGATCACCCCCGGCACCAAC
ACCAGCAACCAGGTGGCCGTGCTGTACCAGGGCGTGAACTGCACCGAGGTGCCCGT
GGCCATCCACGCCGACCAGCTGACCCCCACCTGGAGAGTGTACAGCACCGGCAGCA
ACGTGTTCCAGACCAGAGCCGGCTGCCTGATCGGCGCCGAGCACGTGAACAACAGC
TACGAGTGCGACATCCCCATCGGCGCCGGCATCTGCGCCAGCTACCAGACCCAGAC
CAACAGCCACAGAAGAGCCAGAAGCGTGGCCAGCCAGAGCATCATCGCCTACACCA
TGAGCCTGGGCGCCGAGAACAGCGTGGCCTACAGCAACAACAGCATCGCCATCCCC
ATCAACTTCACCATCAGCGTGACCACCGAGATCCTGCCCGTGAGCATGACCAAGAC
CAGCGTGGACTGCACCATGTACATCTGCGGCGACAGCACCGAGTGCAGCAACCTGC
TGCTGCAGTACGGCAGCTTCTGCACCCAGCTGAACAGAGCCCTGACCGGCATCGCC
GTGGAGCAGGACAAGAACACCCAGGAGGTGTTCGCCCAGGTGAAGCAGATCTACAA
GACCCCCCCCATCAAGGACTTCGGCGGCTTCAACTTCAGCCAGATCCTGCCCGACCC
CAGCAAGCCCAGCAAGAGAAGCTTCATCGAGGACCTGCTGTTCAACAAGGTGACCC
TGGCCGACGCCGGCTTCATCAAGCAGTACGGCGACTGCCTGGGCGACATCGCCGCC
AGAGACCTGATCTGCGCCCAGAAGTTCAACGGCCTGACCGTGCTGCCCCCCCTGCTG
ACCGACGAGATGATCGCCCAGTACACCAGCGCCCTGCTGGCCGGCACCATCACCAG
CGGCTGGACCTTCGGCGCCGGCGCCGCCCTGCAGATCCCCTTCGCCATGCAGATGGC
CTACAGATTCAACGGCATCGGCGTGACCCAGAACGTGCTGTACGAGAACCAGAAGC
TGATCGCCAACCAGTTCAACAGCGCCATCGGCAAGATCCAGGACAGCCTGAGCAGC
ACCGCCAGCGCCCTGGGCAAGCTGCAGGACGTGGTGAACCAGAACGCCCAGGCCCT
GAACACCCTGGTGAAGCAGCTGAGCAGCAACTTCGGCGCCATCAGCAGCGTGCTGA
ACGATATCCTGGCCAGACTGGACCCACCTGAGGCCGAGGTGCAGATCGACAGACTG
ATCACCGGCAGACTGCAGAGCCTGCAGACCTACGTGACCCAGCAGCTGATCAGAGC
CGCCGAGATCAGAGCCAGCGCCAACCTGGCCGCCACCAAGATGAGCGAGTGCGTGC
TGGGCCAGAGCAAGAGAGTGGACTTCTGCGGCAAGGGCTACCACCTGATGAGCTTC
CCCCAGAGCGCCCCCCACGGCGTGGTGTTCCTGCACGTGACCTACGTGCCCGCCCAG
GAGAAGAACTTCACCACCGCCCCCGCCATCTGCCACGACGGCAAGGCCCACTTCCC
CAGAGAGGGCGTGTTCGTGAGCAACGGCACCCACTGGTTCGTGACCCAGAGAAACT
TCTACGAGCCCCAGATCATCACCACCCACAACACCTTCGTGAGCGGCAACTGCGAC
GTGGTGATCGGCATCGTGAACAACACCGTGTACGACCCCCTGCAGCCCGAGCTGGA
CAGCTTCAAGGAGGAGCTGGACAAGTACTTCAAGAACCACACCAGCCCCGACGTGG
ACCTGGGCGACATCAGCGGCATCAACGCCAGCGTGGTGAACATCCAGAAGGAGATC
GACAGACTGAACGAGGTGGCCAAGAATTTAAATGAGAGCCTGATCGACCTGCAGGA
GCTGGGCAAGTACGAGCAGTACATCAAGTGGCCCTGGTACATCTGGCTGGGCTTCAT
CGCCGGCCTGATCGCCATCGTGATGGTGACCATCATGCTGTGCTGCATGACCAGCTG
CTGCAGCTGCCTGAAGGGCTGCTGCAGCTGCGGCAGCTGCTGCAAGTTCGACGAGG
ACGACAGCGAGCCCGTGCTGAAGGGCGTGAAGCTGCACTACACCTAA
SEQ ID NO: 18
MFVFLVLLPLVSSQCVNLTTRTQLPPAYTNSFTRGVYYPDKVFRSSVLHSTQDLFLPFFS
NVTWFHAISGTNGTKRFDNPVLPFNDGVYFASTEKSNIIRGWIFGTTLDSKTQSLLIVNN
ATNVVIKVCEFQFCNDPFLGVYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTEIYQAGSTPCN
GVEGFNCYFPLQSYGFQPTYGVGYQPYRVVVLSFELLHAPATVCGPKKSTNLVKNKCV
NFNFNGLTGTGVLTESNKKFLPFQQFGRDIDDTTDAVRDPQTLEILDITPCSFGGVSVITP
GTNTSNQVAVLYQGVNCTEVPVAIHADQLTPTWRVYSTGSNVFQTRAGCLIGAEHVNN
SYECDIPIGAGICASYQTQTNSHRRARSVASQSIIAYTMSLGAENSVAYSNNSIAIPINFTIS
VTTEILPVSMTKTSVDCTMYICGDSTECSNLLLQYGSFCTQLNRALTGIAVEQDKNTQEV
FAQVKQIYKTPPIKDFGGFNFSQILPDPSKPSKRSFIEDLLFNKVTLADAGFIKQYGDCLG
DIAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQNAQALNT
LVKQLSSNFGAISSVLNDILARLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTHNTFVSGNCDVVIGIVNNTVYDP
LQPELDSFKEELDKYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCSCGSCCKFDEDD
SEPVLKGVKLHYT
SEQ ID NO: 19
ATGTTCGTGTTCCTGGTGCTGCTGCCCCTGGTGAGCAGCCAGTGCGTGAACTTTACC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGCCAACCCCGTGCTGCCCTTCAACGACGGCGTGTACTTCGCCAGCA
CCGAGAAGAGCAACATCATCAGAGGCTGGATCTTCGGCACCACCCTGGACAGCAAG
ACCCAGAGCCTGCTGATCGTGAACAACGCCACCAACGTGGTGATCAAGGTGTGCGA
GTTCCAGTTCTGCAACGACCCCTTCCTGGGCGTGTACTACCACAAGAACAACAAGAG
CTGGATGGAGAGCGAGTTCAGAGTGTACAGCAGCGCCAACAACTGCACCTTCGAGT
ACGTGAGCCAGCCCTTCCTGATGGACCTGGAGGGCAAGCAGGGCAACTTCAAGAAC
CTGAGAGAGTTCGTGTTCAAGAACATCGACGGCTACTTCAAGATCTACAGCAAGCA
CACCCCCATCAACCTGGTGAGAGGCCTGCCCCAGGGCTTCAGCGCCCTGGAGCCCCT
GGTGGACCTGCCCATCGGCATCAACATCACCAGATTCCAGACCCTGCACATTAGCTA
CCTGACCCCCGGCGACAGCAGCAGCGGCTGGACCGCCGGCGCCGCCGCCTACTACG
TGGGCTACCTGCAGCCCAGAACCTTCCTGCTGAAGTACAACGAGAACGGCACCATC
ACCGACGCCGTGGACTGCGCCCTGGACCCCCTGAGCGAGACCAAGTGCACCCTGAA
GAGCTTCACCGTGGAGAAGGGCATCTACCAGACCAGCAACTTCAGAGTGCAGCCCA
CCGAGAGCATCGTGAGATTCCCCAACATCACCAACCTGTGCCCCTTCGGCGAGGTGT
TCAACGCCACCAGATTCGCCAGCGTGTACGCCTGGAACAGAAAGAGAATCAGCAAC
TGCGTGGCCGACTACAGCGTGCTGTACAACAGCGCCAGCTTCAGCACCTTCAAGTGC
TACGGCGTGAGCCCCACCAAGCTGAACGACCTGTGCTTCACCAACGTGTACGCCGA
CAGCTTCGTGATCAGAGGCGACGAGGTGAGACAGATCGCCCCCGGCCAGACCGGCA
ACATCGCCGACTACAACTACAAGCTGCCCGACGACTTCACCGGCTGCGTGATCGCCT
GGAACAGCAACAACCTGGACAGCAAGGTGGGCGGCAACTACAACTACCTGTACAGA
CTGTTCAGAAAGAGCAACCTGAAGCCCTTCGAGAGAGACATCAGCACCGAGATCTA
CCAGGCCGGCAGCACCCCCTGCAACGGCGTGAAGGGCTTCAACTGCTACTTCCCCCT
GCAGAGCTACGGCTTCCAGCCCACCTACGGCGTGGGCTACCAGCCCTACAGAGTGG
TGGTGCTGAGCTTCGAGCTGCTGCACGCCCCCGCCACCGTGTGCGGCCCCAAGAAG
AGCACCAACCTGGTGAAGAACAAGTGCGTGAACTTCAACTTCAACGGCCTGACCGG
CACCGGCGTGCTGACCGAGAGCAACAAGAAGTTCCTGCCCTTCCAGCAGTTCGGCA
GAGACATCGCCGACACCACCGACGCCGTGAGAGACCCCCAGACCCTGGAGATCCTG
GACATCACCCCCTGCAGCTTCGGCGGCGTGAGCGTGATCACCCCCGGCACCAACAC
CAGCAACCAGGTGGCCGTGCTGTACCAGGGCGTGAACTGCACCGAGGTGCCCGTGG
CCATCCACGCCGACCAGCTGACCCCCACCTGGAGAGTGTACAGCACCGGCAGCAAC
GTGTTCCAGACCAGAGCCGGCTGCCTGATCGGCGCCGAGCACGTGAACAACAGCTA
CGAGTGCGACATCCCCATCGGCGCCGGCATCTGCGCCAGCTACCAGACCCAGACCA
ACAGCCCCAGAAGAGCCAGAAGCGTGGCCAGCCAGAGCATCATCGCCTACACCATG
AGCCTGGGCGTGGAGAACAGCGTGGCCTACAGCAACAACAGCATCGCCATCCCCAC
CAACTTCACCATCAGCGTGACCACCGAGATCCTGCCCGTGAGCATGACCAAGACCA
GCGTGGACTGCACCATGTACATCTGCGGCGACAGCACCGAGTGCAGCAACCTGCTG
CTGCAGTACGGCAGCTTCTGCACCCAGCTGAACAGAGCCCTGACCGGCATCGCCGT
GGAGCAGGACAAGAACACCCAGGAGGTGTTCGCCCAGGTGAAGCAGATCTACAAG
ACCCCCCCCATCAAGGACTTCGGCGGCTTCAACTTCAGCCAGATCCTGCCCGACCCC
AGCAAGCCCAGCAAGAGAAGCTTCATCGAGGACCTGCTGTTCAACAAGGTGACCCT
GGCCGACGCCGGCTTCATCAAGCAGTACGGCGACTGCCTGGGCGACATCGCCGCCA
GAGACCTGATCTGCGCCCAGAAGTTCAACGGCCTGACCGTGCTGCCCCCCCTGCTGA
CCGACGAGATGATCGCCCAGTACACCAGCGCCCTGCTGGCCGGCACCATCACCAGC
GGCTGGACCTTCGGCGCCGGCGCCGCCCTGCAGATCCCCTTCGCCATGCAGATGGCC
TACAGATTCAACGGCATCGGCGTGACCCAGAACGTGCTGTACGAGAACCAGAAGCT
GATCGCCAACCAGTTCAACAGCGCCATCGGCAAGATCCAGGACAGCCTGAGCAGCA
CCGCCAGCGCCCTGGGCAAGCTGCAGGACGTGGTGAACCAGAACGCCCAGGCCCTG
AACACCCTGGTGAAGCAGCTGAGCAGCAACTTCGGCGCCATCAGCAGCGTGCTGAA
CGATATCCTGAGCAGACTGGACCCACCTGAGGCCGAGGTGCAGATCGACAGACTGA
TCACCGGCAGACTGCAGAGCCTGCAGACCTACGTGACCCAGCAGCTGATCAGAGCC
GCCGAGATCAGAGCCAGCGCCAACCTGGCCGCCACCAAGATGAGCGAGTGCGTGCT
GGGCCAGAGCAAGAGAGTGGACTTCTGCGGCAAGGGCTACCACCTGATGAGCTTCC
CCCAGAGCGCCCCCCACGGCGTGGTGTTCCTGCACGTGACCTACGTGCCCGCCCAGG
AGAAGAACTTCACCACCGCCCCCGCCATCTGCCACGACGGCAAGGCCCACTTCCCC
AGAGAGGGCGTGTTCGTGAGCAACGGCACCCACTGGTTCGTGACCCAGAGAAACTT
CTACGAGCCCCAGATCATCACCACCGACAACACCTTCGTGAGCGGCAACTGCGACG
TGGTGATCGGCATCGTGAACAACACCGTGTACGACCCCCTGCAGCCCGAGCTGGAC
AGCTTCAAGGAGGAGCTGGACAAGTACTTCAAGAACCACACCAGCCCCGACGTGGA
CCTGGGCGACATCAGCGGCATCAACGCCAGCGTGGTGAACATCCAGAAGGAGATCG
ACAGACTGAACGAGGTGGCCAAGAATTTAAATGAGAGCCTGATCGACCTGCAGGAG
CTGGGCAAGTACGAGCAGTACATCAAGTGGCCCTGGTACATCTGGCTGGGCTTCATC
GCCGGCCTGATCGCCATCGTGATGGTGACCATCATGCTGTGCTGCATGACCAGCTGC
TGCAGCTGCCTGAAGGGCTGCTGCAGCTGCGGCAGCTGCTGCAAGTTCGACGAGGA
CGACAGCGAGCCCGTGCTGAAGGGCGTGAAGCTGCACTACACCTAA
SEQ ID NO: 20
MFVFLVLLPLVSSQCVNFTTRTQLPPAYTNSFTRGVYYPDKVFRSSVLHSTQDLFLPFFS
NVTWFHAIHVSGTNGTKRFANPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRGLPQGFSALEPLVDLPIGINITRFQT
LHISYLTPGDSSSGWTAGAAAYYVGYLQPRTFLLKYNENGTITDAVDCALDPLSETKCT
LKSFTVEKGIYQTSNFRVQPTESIVRFPNITNLCPFGEVENATRFASVYAWNRKRISNCVA
DYSVLYNSASFSTFKCYGVSPTKLNDLCFTNVYADSFVIRGDEVRQIAPGQTGNIADYN
YKLPDDFTGCVIAWNSNNLDSKVGGNYNYLYRLFRKSNLKPFERDISTEIYQAGSTPCN
GVKGFNCYFPLQSYGFQPTYGVGYQPYRVVVLSFELLHAPATVCGPKKSTNLVKNKCV
NFNFNGLTGTGVLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCLIGAEHVNN
SYECDIPIGAGICASYQTQTNSPRRARSVASQSIIAYTMSLGVENSVAYSNNSIAIPTNFTIS
VTTEILPVSMTKTSVDCTMYICGDSTECSNLLLQYGSFCTQLNRALTGIAVEQDKNTQEV
FAQVKQIYKTPPIKDFGGFNFSQILPDPSKPSKRSFIEDLLFNKVTLADAGFIKQYGDCLG
DIAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQNAQALNT
LVKQLSSNFGAISSVLNDILSRLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCSCGSCCKFDEDD
SEPVLKGVKLHYT
SEQ ID NO: 21-Coro VaxG.3-P spike protein-nucleic acid sequence
ATGTTCGTGTTCCTGGTGCTGCTGCCCCTGGTGAGCAGCCAGTGCGTGAACTTCACC
AACAGAACCCAGCTGCCCAGCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGACAACCCCGTGCTGCCCTTCAACGACGGCGTGTACTTCGCCAGCA
CCGAGAAGAGCAACATCATCAGAGGCTGGATCTTCGGCACCACCCTGGACAGCAAG
ACCCAGAGCCTGCTGATCGTGAACAACGCCACCAACGTGGTGATCAAGGTGTGCGA
GTTCCAGTTCTGCAACTACCCCTTCCTGGGCGTGTACTACCACAAGAACAACAAGAG
CTGGATGGAGAGCGAGTTCAGAGTGTACAGCAGCGCCAACAACTGCACCTTCGAGT
ACGTGAGCCAGCCCTTCCTGATGGACCTGGAGGGCAAGCAGGGCAACTTCAAGAAC
CTGAGCGAGTTCGTGTTCAAGAACATCGACGGCTACTTCAAGATCTACAGCAAGCAC
ACCCCCATCAACCTGGTGAGAGACCTGCCCCAGGGCTTCAGCGCCCTGGAGCCCCTG
GTGGACCTGCCCATCGGCATCAACATCACCAGATTCCAGACCCTGCTGGCCCTGCAC
AGAAGCTACCTGACCCCCGGCGACAGCAGCAGCGGCTGGACCGCCGGCGCCGCCGC
CTACTACGTGGGCTACCTGCAGCCCAGAACCTTCCTGCTGAAGTACAACGAGAACG
GCACCATCACCGACGCCGTGGACTGCGCCCTGGACCCCCTGAGCGAGACCAAGTGC
ACCCTGAAGAGCTTCACCGTGGAGAAGGGCATCTACCAGACCAGCAACTTCAGAGT
GCAGCCCACCGAGAGCATCGTGAGATTCCCCAACATCACCAACCTGTGCCCCTTCGG
CGAGGTGTTCAACGCCACCAGATTCGCCAGCGTGTACGCCTGGAACAGAAAGAGAA
TCAGCAACTGCGTGGCCGACTACAGCGTGCTGTACAACAGCGCCAGCTTCAGCACCT
TCAAGTGCTACGGCGTGAGCCCCACCAAGCTGAACGACCTGTGCTTCACCAACGTGT
ACGCCGACAGCTTCGTGATCAGAGGCGACGAGGTGAGACAGATCGCCCCCGGCCAG
ACCGGCACCATCGCCGACTACAACTACAAGCTGCCCGACGACTTCACCGGCTGCGT
GATCGCCTGGAACAGCAACAACCTGGACAGCAAGGTGGGCGGCAACTACAACTACC
TGTACAGACTGTTCAGAAAGAGCAACCTGAAGCCCTTCGAGAGAGACATCAGCACC
GAGATCTACCAGGCCGGCAGCACCCCCTGCAACGGCGTGAAGGGCTTCAACTGCTA
CTTCCCCCTGCAGAGCTACGGCTTCCAGCCCACCTACGGCGTGGGCTACCAGCCCTA
CAGAGTGGTGGTGCTGAGCTTCGAGCTGCTGCACGCCCCCGCCACCGTGTGCGGCCC
CAAGAAGAGCACCAACCTGGTGAAGAACAAGTGCGTGAACTTCAACTTCAACGGCC
TGACCGGCACCGGCGTGCTGACCGAGAGCAACAAGAAGTTCCTGCCCTTCCAGCAG
TTCGGCAGAGACATCGCCGACACCACCGACGCCGTGAGAGACCCCCAGACCCTGGA
GATCCTGGACATCACCCCCTGCAGCTTCGGCGGCGTGAGCGTGATCACCCCCGGCAC
CAACACCAGCAACCAGGTGGCCGTGCTGTACCAGGGCGTGAACTGCACCGAGGTGC
CCGTGGCCATCCACGCCGACCAGCTGACCCCCACCTGGAGAGTGTACAGCACCGGC
AGCAACGTGTTCCAGACCAGAGCCGGCTGCCTGATCGGCGCCGAGTACGTGAACAA
CAGCTACGAGTGCGACATCCCCATCGGCGCCGGCATCTGCGCCAGCTACCAGACCC
AGACCAACAGCCCCAGAAGAGCCAGAAGCGTGGCCAGCCAGAGCATCATCGCCTAC
ACCATGAGCCTGGGCGCCGAGAACAGCGTGGCCTACAGCAACAACAGCATCGCCAT
CCCCACCAACTTCACCATCAGCGTGACCACCGAGATCCTGCCCGTGAGCATGACCAA
GACCAGCGTGGACTGCACCATGTACATCTGCGGCGACAGCACCGAGTGCAGCAACC
TGCTGCTGCAGTACGGCAGCTTCTGCACCCAGCTGAACAGAGCCCTGACCGGCATCG
CCGTGGAGCAGGACAAGAACACCCAGGAGGTGTTCGCCCAGGTGAAGCAGATCTAC
AAGACCCCCCCCATCAAGGACTTCGGCGGCTTCAACTTCAGCCAGATCCTGCCCGAC
CCCAGCAAGCCCAGCAAGAGAAGCTTCATCGAGGACCTGCTGTTCAACAAGGTGAC
CCTGGCCGACGCCGGCTTCATCAAGCAGTACGGCGACTGCCTGGGCGACATCGCCG
CCAGAGACCTGATCTGCGCCCAGAAGTTCAACGGCCTGACCGTGCTGCCCCCCCTGC
TGACCGACGAGATGATCGCCCAGTACACCAGCGCCCTGCTGGCCGGCACCATCACC
AGCGGCTGGACCTTCGGCGCCGGCGCCGCCCTGCAGATCCCCTTCGCCATGCAGATG
GCCTACAGATTCAACGGCATCGGCGTGACCCAGAACGTGCTGTACGAGAACCAGAA
GCTGATCGCCAACCAGTTCAACAGCGCCATCGGCAAGATCCAGGACAGCCTGAGCA
GCACCGCCAGCGCCCTGGGCAAGCTGCAGGACGTGGTGAACCAGAACGCCCAGGCC
CTGAACACCCTGGTGAAGCAGCTGAGCAGCAACTTCGGCGCCATCAGCAGCGTGCT
GAACGATATCCTGAGCAGACTGGACCCACCTGAGGCCGAGGTGCAGATCGACAGAC
TGATCACCGGCAGACTGCAGAGCCTGCAGACCTACGTGACCCAGCAGCTGATCAGA
GCCGCCGAGATCAGAGCCAGCGCCAACCTGGCCGCCATCAAGATGAGCGAGTGCGT
GCTGGGCCAGAGCAAGAGAGTGGACTTCTGCGGCAAGGGCTACCACCTGATGAGCT
TCCCCCAGAGCGCCCCCCACGGCGTGGTGTTCCTGCACGTGACCTACGTGCCCGCCC
AGGAGAAGAACTTCACCACCGCCCCCGCCATCTGCCACGACGGCAAGGCCCACTTC
CCCAGAGAGGGCGTGTTCGTGAGCAACGGCACCCACTGGTTCGTGACCCAGAGAAA
CTTCTACGAGCCCCAGATCATCACCACCGACAACACCTTCGTGAGCGGCAACTGCGA
CGTGGTGATCGGCATCGTGAACAACACCGTGTACGACCCCCTGCAGCCCGAGCTGG
ACAGCTTCAAGGAGGAGCTGGACAAGTACTTCAAGAACCACACCAGCCCCGACGTG
GACCTGGGCGACATCAGCGGCATCAACGCCAGCTTCGTGAACATCCAGAAGGAGAT
CGACAGACTGAACGAGGTGGCCAAGAATTTAAATGAGAGCCTGATCGACCTGCAGG
AGCTGGGCAAGTACGAGCAGTACATCAAGTGGCCCTGGTACATCTGGCTGGGCTTC
ATCGCCGGCCTGATCGCCATCGTGATGGTGACCATCATGCTGTGCTGCATGACCAGC
TGCTGCAGCTGCCTGAAGGGCTGCTGCAGCTGCGGCAGCTGCTGCAAGTTCGACGA
GGACGACAGCGAGCCCGTGCTGAAGGGCGTGAAGCTGCACTACACCTAA
SEQ ID NO: 22-CoroVaxG.3-P spike protein-amino acid sequence
MFVFLVLLPLVSSQCVNFTNRTQLPSAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNYPFLGVYYHKNNKSWMESEFRVYSSANNCTFEYVSQPFLMD
LEGKQGNFKNLSEFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVENATRFASVYAWNRKRISN
CVADYSVLYNSASFSTFKCYGVSPTKLNDLCFTNVYADSFVIRGDEVRQIAPGQTGTIAD
YNYKLPDDFTGCVIAWNSNNLDSKVGGNYNYLYRLFRKSNLKPFERDISTEIYQAGSTP
CNGVKGFNCYFPLQSYGFQPTYGVGYQPYRVVVLSFELLHAPATVCGPKKSTNLVKNK
CVNFNFNGLTGTGVLTESNKKFLPFQQFGRDIADTTDAVRDPQTLEILDITPCSFGGVSVI
TPGTNTSNQVAVLYQGVNCTEVPVAIHADQLTPTWRVYSTGSNVFQTRAGCLIGAEYV
NNSYECDIPIGAGICASYQTQTNSPRRARSVASQSIIAYTMSLGAENSVAYSNNSIAIPTNF
TISVTTEILPVSMTKTSVDCTMYICGDSTECSNLLLQYGSFCTQLNRALTGIAVEQDKNT
QEVFAQVKQIYKTPPIKDFGGFNFSQILPDPSKPSKRSFIEDLLFNKVTLADAGFIKQYGD
CLGDIAARDLICAQKFNGLTVLPPLLTDEMIAQYTSALLAGTITSGWTFGAGAALQIPFA
MQMAYRFNGIGVTQNVLYENQKLIANQFNSAIGKIQDSLSSTASALGKLQDVVNQNAQ
ALNTLVKQLSSNFGAISSVLNDILSRLDPPEAEVQIDRLITGRLQSLQTYVTQQLIRAAEIR
ASANLAAIKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNNTV
YDPLQPELDSFKEELDKYFKNHTSPDVDLGDISGINASFVNIQKEIDRLNEVAKNLNESLI
DLQELGKYEQYIKWPWYIWLGFIAGLIAIVMVTIMLCCMTSCCSCLKGCCSCGSCCKFD
EDDSEPVLKGVKLHYT
SEQ ID NO: 23-CoroVaxG.3-D spike protein-nucleic acid sequence
ATGTTCGTGTTCCTGGTGCTGCTGCCCCTGGTGAGCAGCCAGTGCGTGAACCTGCGC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGACAACCCCGTGCTGCCCTTCAACGACGGCGTGTACTTCGCCAGCA
CCGAGAAGAGCAACATCATCAGAGGCTGGATCTTCGGCACCACCCTGGACAGCAAG
ACCCAGAGCCTGCTGATCGTGAACAACGCCACCAACGTGGTGATCAAGGTGTGCGA
GTTCCAGTTCTGCAACGACCCCTTCCTGGGCGTGTACTACCACAAGAACAACAAGAG
CTGGATGGAGAGCGGAGTGTACAGCAGCGCCAACAACTGCACCTTCGAGTACGTGA
GCCAGCCCTTCCTGATGGACCTGGAGGGCAAGCAGGGCAACTTCAAGAACCTGAGA
GAGTTCGTGTTCAAGAACATCGACGGCTACTTCAAGATCTACAGCAAGCACACCCCC
ATCAACCTGGTGAGAGACCTGCCCCAGGGCTTCAGCGCCCTGGAGCCCCTGGTGGA
CCTGCCCATCGGCATCAACATCACCAGATTCCAGACCCTGCTGGCCCTGCACAGAAG
CTACCTGACCCCCGGCGACAGCAGCAGCGGCTGGACCGCCGGCGCCGCCGCCTACT
ACGTGGGCTACCTGCAGCCCAGAACCTTCCTGCTGAAGTACAACGAGAACGGCACC
ATCACCGACGCCGTGGACTGCGCCCTGGACCCCCTGAGCGAGACCAAGTGCACCCT
GAAGAGCTTCACCGTGGAGAAGGGCATCTACCAGACCAGCAACTTCAGAGTGCAGC
CCACCGAGAGCATCGTGAGATTCCCCAACATCACCAACCTGTGCCCCTTCGGCGAGG
TGTTCAACGCCACCAGATTCGCCAGCGTGTACGCCTGGAACAGAAAGAGAATCAGC
AACTGCGTGGCCGACTACAGCGTGCTGTACAACAGCGCCAGCTTCAGCACCTTCAA
GTGCTACGGCGTGAGCCCCACCAAGCTGAACGACCTGTGCTTCACCAACGTGTACGC
CGACAGCTTCGTGATCAGAGGCGACGAGGTGAGACAGATCGCCCCCGGCCAGACCG
GCAACATCGCCGACTACAACTACAAGCTGCCCGACGACTTCACCGGCTGCGTGATC
GCCTGGAACAGCAACAACCTGGACAGCAAGGTGGGCGGCAACTACAACTACCGGTA
CAGACTGTTCAGAAAGAGCAACCTGAAGCCCTTCGAGAGAGACATCAGCACCGAGA
TCTACCAGGCCGGCAGCAAGCCCTGCAACGGCGTGGAGGGCTTCAACTGCTACTTCC
CCCTGCAGAGCTACGGCTTCCAGCCCACCAACGGCGTGGGCTACCAGCCCTACAGA
GTGGTGGTGCTGAGCTTCGAGCTGCTGCACGCCCCCGCCACCGTGTGCGGCCCCAAG
AAGAGCACCAACCTGGTGAAGAACAAGTGCGTGAACTTCAACTTCAACGGCCTGAC
CGGCACCGGCGTGCTGACCGAGAGCAACAAGAAGTTCCTGCCCTTCCAGCAGTTCG
GCAGAGACATCGCCGACACCACCGACGCCGTGAGAGACCCCCAGACCCTGGAGATC
CTGGACATCACCCCCTGCAGCTTCGGCGGCGTGAGCGTGATCACCCCCGGCACCAAC
ACCAGCAACCAGGTGGCCGTGCTGTACCAGGGCGTGAACTGCACCGAGGTGCCCGT
GGCCATCCACGCCGACCAGCTGACCCCCACCTGGAGAGTGTACAGCACCGGCAGCA
ACGTGTTCCAGACCAGAGCCGGCTGCCTGATCGGCGCCGAGCACGTGAACAACAGC
TACGAGTGCGACATCCCCATCGGCGCCGGCATCTGCGCCAGCTACCAGACCCAGAC
CAACAGCCGGAGAAGAGCCAGAAGCGTGGCCAGCCAGAGCATCATCGCCTACACCA
TGAGCCTGGGCGCCGAGAACAGCGTGGCCTACAGCAACAACAGCATCGCCATCCCC
ACCAACTTCACCATCAGCGTGACCACCGAGATCCTGCCCGTGAGCATGACCAAGAC
CAGCGTGGACTGCACCATGTACATCTGCGGCGACAGCACCGAGTGCAGCAACCTGC
TGCTGCAGTACGGCAGCTTCTGCACCCAGCTGAACAGAGCCCTGACCGGCATCGCC
GTGGAGCAGGACAAGAACACCCAGGAGGTGTTCGCCCAGGTGAAGCAGATCTACAA
GACCCCCCCCATCAAGGACTTCGGCGGCTTCAACTTCAGCCAGATCCTGCCCGACCC
CAGCAAGCCCAGCAAGAGAAGCTTCATCGAGGACCTGCTGTTCAACAAGGTGACCC
TGGCCGACGCCGGCTTCATCAAGCAGTACGGCGACTGCCTGGGCGACATCGCCGCC
AGAGACCTGATCTGCGCCCAGAAGTTCAACGGCCTGACCGTGCTGCCCCCCCTGCTG
ACCGACGAGATGATCGCCCAGTACACCAGCGCCCTGCTGGCCGGCACCATCACCAG
CGGCTGGACCTTCGGCGCCGGCGCCGCCCTGCAGATCCCCTTCGCCATGCAGATGGC
CTACAGATTCAACGGCATCGGCGTGACCCAGAACGTGCTGTACGAGAACCAGAAGC
TGATCGCCAACCAGTTCAACAGCGCCATCGGCAAGATCCAGGACAGCCTGAGCAGC
ACCGCCAGCGCCCTGGGCAAGCTGCAGAACGTGGTGAACCAGAACGCCCAGGCCCT
GAACACCCTGGTGAAGCAGCTGAGCAGCAACTTCGGCGCCATCAGCAGCGTGCTGA
ACGATATCCTGAGCAGACTGGACCCACCTGAGGCCGAGGTGCAGATCGACAGACTG
ATCACCGGCAGACTGCAGAGCCTGCAGACCTACGTGACCCAGCAGCTGATCAGAGC
CGCCGAGATCAGAGCCAGCGCCAACCTGGCCGCCACCAAGATGAGCGAGTGCGTGC
TGGGCCAGAGCAAGAGAGTGGACTTCTGCGGCAAGGGCTACCACCTGATGAGCTTC
CCCCAGAGCGCCCCCCACGGCGTGGTGTTCCTGCACGTGACCTACGTGCCCGCCCAG
GAGAAGAACTTCACCACCGCCCCCGCCATCTGCCACGACGGCAAGGCCCACTTCCC
CAGAGAGGGCGTGTTCGTGAGCAACGGCACCCACTGGTTCGTGACCCAGAGAAACT
TCTACGAGCCCCAGATCATCACCACCGACAACACCTTCGTGAGCGGCAACTGCGAC
GTGGTGATCGGCATCGTGAACAACACCGTGTACGACCCCCTGCAGCCCGAGCTGGA
CAGCTTCAAGGAGGAGCTGGACAAGTACTTCAAGAACCACACCAGCCCCGACGTGG
ACCTGGGCGACATCAGCGGCATCAACGCCAGCGTGGTGAACATCCAGAAGGAGATC
GACAGACTGAACGAGGTGGCCAAGAATTTAAATGAGAGCCTGATCGACCTGCAGGA
GCTGGGCAAGTACGAGCAGTACATCAAGTGGCCCTGGTACATCTGGCTGGGCTTCAT
CGCCGGCCTGATCGCCATCGTGATGGTGACCATCATGCTGTGCTGCATGACCAGCTG
CTGCAGCTGCCTGAAGGGCTGCTGCAGCTGCGGCAGCTGCTGCAAGTTCGACGAGG
ACGACAGCGAGCCCGTGCTGAAGGGCGTGAAGCTGCACTACACTA
SEQ ID NO: 24-CoroVaxG.3-D spike protein-amino acid sequence
MFVFLVLLPLVSSQCVNLRTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESGVYSSANNCTFEYVSQPFLMDLE
GKQGNFKNLREFVFKNIDGYFKIYSKHTPINLVRDLPQGFSALEPLVDLPIGINITRFQTLL
ALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASVYAWNRKRISNC
VADYSVLYNSASFSTFKCYGVSPTKLNDLCFTNVYADSFVIRGDEVRQIAPGQTGNIAD
YNYKLPDDFTGCVIAWNSNNLDSKVGGNYNYRYRLFRKSNLKPFERDISTEIYQAGSKP
CNGVEGFNCYFPLQSYGFQPTNGVGYQPYRVVVLSFELLHAPATVCGPKKSTNLVKNK
CVNFNFNGLTGTGVLTESNKKFLPFQQFGRDIADTTDAVRDPQTLEILDITPCSFGGVSVI
TPGTNTSNQVAVLYQGVNCTEVPVAIHADQLTPTWRVYSTGSNVFQTRAGCLIGAEHV
NNSYECDIPIGAGICASYQTQTNSRRRARSVASQSIIAYTMSLGAENSVAYSNNSIAIPTNF
TISVTTEILPVSMTKTSVDCTMYICGDSTECSNLLLQYGSFCTQLNRALTGIAVEQDKNT
QEVFAQVKQIYKTPPIKDFGGFNFSQILPDPSKPSKRSFIEDLLFNKVTLADAGFIKQYGD
CLGDIAARDLICAQKFNGLTVLPPLLTDEMIAQYTSALLAGTITSGWTFGAGAALQIPFA
MQMAYRENGIGVTQNVLYENQKLIANQFNSAIGKIQDSLSSTASALGKLQNVVNQNAQ
ALNTLVKQLSSNFGAISSVLNDILSRLDPPEAEVQIDRLITGRLQSLQTYVTQQLIRAAEIR
ASANLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNEVAKNLNESLI
DLQELGKYEQYIKWPWYIWLGFIAGLIAIVMVTIMLCCMTSCCSCLKGCCSCGSCCKED
EDDSEPVLKGVKLHYT
SEQ ID NO: 25-CorovaxG.3-D.FR-nucleic acid sequence
ATGTTCGTGTTCCTGGTGCTGCTGCCCCTGGTGAGCAGCCAGTGCGTGAACCTGCGC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGCCATCCACGTGAGCGGCACCAACGGCA
CCAAGAGATTCGACAACCCCGTGCTGCCCTTCAACGACGGCGTGTACTTCGCCAGCA
CCGAGAAGAGCAACATCATCAGAGGCTGGATCTTCGGCACCACCCTGGACAGCAAG
ACCCAGAGCCTGCTGATCGTGAACAACGCCACCAACGTGGTGATCAAGGTGTGCGA
GTTCCAGTTCTGCAACGACCCCTTCCTGGGCGTGTACTACCACAAGAACAACAAGAG
CTGGATGGAGAGCGGAGTGTACAGCAGCGCCAACAACTGCACCTTCGAGTACGTGA
GCCAGCCCTTCCTGATGGACCTGGAGGGCAAGCAGGGCAACTTCAAGAACCTGAGA
GAGTTCGTGTTCAAGAACATCGACGGCTACTTCAAGATCTACAGCAAGCACACCCCC
ATCAACCTGGTGAGAGACCTGCCCCAGGGCTTCAGCGCCCTGGAGCCCCTGGTGGA
CCTGCCCATCGGCATCAACATCACCAGATTCCAGACCCTGCTGGCCCTGCACAGAAG
CTACCTGACCCCCGGCGACAGCAGCAGCGGCTGGACCGCCGGCGCCGCCGCCTACT
ACGTGGGCTACCTGCAGCCCAGAACCTTCCTGCTGAAGTACAACGAGAACGGCACC
ATCACCGACGCCGTGGACTGCGCCCTGGACCCCCTGAGCGAGACCAAGTGCACCCT
GAAGAGCTTCACCGTGGAGAAGGGCATCTACCAGACCAGCAACTTCAGAGTGCAGC
CCACCGAGAGCATCGTGAGATTCCCCAACATCACCAACCTGTGCCCCTTCGGCGAGG
TGTTCAACGCCACCAGATTCGCCAGCGTGTACGCCTGGAACAGAAAGAGAATCAGC
AACTGCGTGGCCGACTACAGCGTGCTGTACAACAGCGCCAGCTTCAGCACCTTCAA
GTGCTACGGCGTGAGCCCCACCAAGCTGAACGACCTGTGCTTCACCAACGTGTACGC
CGACAGCTTCGTGATCAGAGGCGACGAGGTGAGACAGATCGCCCCCGGCCAGACCG
GCAACATCGCCGACTACAACTACAAGCTGCCCGACGACTTCACCGGCTGCGTGATC
GCCTGGAACAGCAACAACCTGGACAGCAAGGTGGGCGGCAACTACAACTACCGGTA
CAGACTGTTCAGAAAGAGCAACCTGAAGCCCTTCGAGAGAGACATCAGCACCGAGA
TCTACCAGGCCGGCAGCAAGCCCTGCAACGGCGTGGAGGGCTTCAACTGCTACTTCC
CCCTGCAGAGCTACGGCTTCCAGCCCACCAACGGCGTGGGCTACCAGCCCTACAGA
GTGGTGGTGCTGAGCTTCGAGCTGCTGCACGCCCCCGCCACCGTGTGCGGCCCCAAG
AAGAGCACCAACCTGGTGAAGAACAAGTGCGTGAACTTCAACTTCAACGGCCTGAC
CGGCACCGGCGTGCTGACCGAGAGCAACAAGAAGTTCCTGCCCTTCCAGCAGTTCG
GCAGAGACATCGCCGACACCACCGACGCCGTGAGAGACCCCCAGACCCTGGAGATC
CTGGACATCACCCCCTGCAGCTTCGGCGGCGTGAGCGTGATCACCCCCGGCACCAAC
ACCAGCAACCAGGTGGCCGTGCTGTACCAGGGCGTGAACTGCACCGAGGTGCCCGT
GGCCATCCACGCCGACCAGCTGACCCCCACCTGGAGAGTGTACAGCACCGGCAGCA
ACGTGTTCCAGACCAGAGCCGGCTGCCTGATCGGCGCCGAGCACGTGAACAACAGC
TACGAGTGCGACATCCCCATCGGCGCCGGCATCTGCGCCAGCTACCAGACCCAGAC
CAACAGCCGGGGAAGCGCCAGCAGCGTGGCCAGCCAGAGCATCATCGCCTACACCA
TGAGCCTGGGCGCCGAGAACAGCGTGGCCTACAGCAACAACAGCATCGCCATCCCC
ACCAACTTCACCATCAGCGTGACCACCGAGATCCTGCCCGTGAGCATGACCAAGAC
CAGCGTGGACTGCACCATGTACATCTGCGGCGACAGCACCGAGTGCAGCAACCTGC
TGCTGCAGTACGGCAGCTTCTGCACCCAGCTGAACAGAGCCCTGACCGGCATCGCC
GTGGAGCAGGACAAGAACACCCAGGAGGTGTTCGCCCAGGTGAAGCAGATCTACAA
GACCCCCCCCATCAAGGACTTCGGCGGCTTCAACTTCAGCCAGATCCTGCCCGACCC
CAGCAAGCCCAGCAAGAGAAGCTTCATCGAGGACCTGCTGTTCAACAAGGTGACCC
TGGCCGACGCCGGCTTCATCAAGCAGTACGGCGACTGCCTGGGCGACATCGCCGCC
AGAGACCTGATCTGCGCCCAGAAGTTCAACGGCCTGACCGTGCTGCCCCCCCTGCTG
ACCGACGAGATGATCGCCCAGTACACCAGCGCCCTGCTGGCCGGCACCATCACCAG
CGGCTGGACCTTCGGCGCCGGCGCCGCCCTGCAGATCCCCTTCGCCATGCAGATGGC
CTACAGATTCAACGGCATCGGCGTGACCCAGAACGTGCTGTACGAGAACCAGAAGC
TGATCGCCAACCAGTTCAACAGCGCCATCGGCAAGATCCAGGACAGCCTGAGCAGC
ACCGCCAGCGCCCTGGGCAAGCTGCAGAACGTGGTGAACCAGAACGCCCAGGCCCT
GAACACCCTGGTGAAGCAGCTGAGCAGCAACTTCGGCGCCATCAGCAGCGTGCTGA
ACGATATCCTGAGCAGACTGGACCCACCTGAGGCCGAGGTGCAGATCGACAGACTG
ATCACCGGCAGACTGCAGAGCCTGCAGACCTACGTGACCCAGCAGCTGATCAGAGC
CGCCGAGATCAGAGCCAGCGCCAACCTGGCCGCCACCAAGATGAGCGAGTGCGTGC
TGGGCCAGAGCAAGAGAGTGGACTTCTGCGGCAAGGGCTACCACCTGATGAGCTTC
CCCCAGAGCGCCCCCCACGGCGTGGTGTTCCTGCACGTGACCTACGTGCCCGCCCAG
GAGAAGAACTTCACCACCGCCCCCGCCATCTGCCACGACGGCAAGGCCCACTTCCC
CAGAGAGGGCGTGTTCGTGAGCAACGGCACCCACTGGTTCGTGACCCAGAGAAACT
TCTACGAGCCCCAGATCATCACCACCGACAACACCTTCGTGAGCGGCAACTGCGAC
GTGGTGATCGGCATCGTGAACAACACCGTGTACGACCCCCTGCAGCCCGAGCTGGA
CAGCTTCAAGGAGGAGCTGGACAAGTACTTCAAGAACCACACCAGCCCCGACGTGG
ACCTGGGCGACATCAGCGGCATCAACGCCAGCGTGGTGAACATCCAGAAGGAGATC
GACAGACTGAACGAGGTGGCCAAGAATTTAAATGAGAGCCTGATCGACCTGCAGGA
GCTGGGCAAGTACGAGCAGTACATCAAGTGGCCCTGGTACATCTGGCTGGGCTTCAT
CGCCGGCCTGATCGCCATCGTGATGGTGACCATCATGCTGTGCTGCATGACCAGCTG
CTGCAGCTGCCTGAAGGGCTGCTGCAGCTGCGGCAGCTGCTGCAAGTTCGACGAGG
ACGACAGCGAGCCCGTGCTGAAGGGCGTGAAGCTGCACTACACCTAA
SEQ ID NO: 26-CorovaxG.3-D.FR-amino acid sequence
MFVFLVLLPLVSSQCVNLRTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESGVYSSANNCTFEYVSQPFLMDLE
GKQGNFKNLREFVFKNIDGYFKIYSKHTPINLVRDLPQGFSALEPLVDLPIGINITRFQTLL
ALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGNIAD
YNYKLPDDFTGCVIAWNSNNLDSKVGGNYNYRYRLFRKSNLKPFERDISTEIYQAGSKP
CNGVEGFNCYFPLQSYGFQPTNGVGYQPYRVVVLSFELLHAPATVCGPKKSTNLVKNK
CVNFNFNGLTGTGVLTESNKKFLPFQQFGRDIADTTDAVRDPQTLEILDITPCSFGGVSVI
TPGTNTSNQVAVLYQGVNCTEVPVAIHADQLTPTWRVYSTGSNVFQTRAGCLIGAEHV
NNSYECDIPIGAGICASYQTQTNSRGSASSVASQSIIAYTMSLGAENSVAYSNNSIAIPTNF
TISVTTEILPVSMTKTSVDCTMYICGDSTECSNLLLQYGSFCTQLNRALTGIAVEQDKNT
QEVFAQVKQIYKTPPIKDFGGFNFSQILPDPSKPSKRSFIEDLLFNKVTLADAGFIKQYGD
CLGDIAARDLICAQKFNGLTVLPPLLTDEMIAQYTSALLAGTITSGWTFGAGAALQIPFA
MQMAYRFNGIGVTQNVLYENQKLIANQFNSAIGKIQDSLSSTASALGKLQNVVNQNAQ
ALNTLVKQLSSNFGAISSVLNDILSRLDPPEAEVQIDRLITGRLQSLQTYVTQQLIRAAEIR
ASANLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNEVAKNLNESLI
DLQELGKYEQYIKWPWYIWLGFIAGLIAIVMVTIMLCCMTSCCSCLKGCCSCGSCCKED
EDDSEPVLKGVKLHYT
SEQ ID NO: 27-CorovaxG.3-O.FR-nucleic acid sequence
ATGTTCGTGTTCCTGGTGCTGCTGCCCCTGGTGAGCAGCCAGTGCGTGAACCTGACC
ACCAGAACCCAGCTGCCCCCCGCCTACACCAACAGCTTCACCAGAGGCGTGTACTA
CCCCGACAAGGTGTTCAGAAGCAGCGTGCTGCACAGCACCCAGGACCTGTTCCTGC
CCTTCTTCAGCAACGTGACCTGGTTCCACGTGATCAGCGGCACCAACGGCACCAAGA
GATTCGACAACCCCGTGCTGCCCTTCAACGACGGCGTGTACTTCGCCAGCATCGAGA
AGAGCAACATCATCAGAGGCTGGATCTTCGGCACCACCCTGGACAGCAAGACCCAG
AGCCTGCTGATCGTGAACAACGCCACCAACGTGGTGATCAAGGTGTGCGAGTTCCA
GTTCTGCAACGACCCCTTCCTGGACCACAAGAACAACAAGAGCTGGATGGAGAGCG
AGTTCAGAGTGTACAGCAGCGCCAACAACTGCACCTTCGAGTACGTGAGCCAGCCC
TTCCTGATGGACCTGGAGGGCAAGCAGGGCAACTTCAAGAACCTGAGAGAGTTCGT
GTTCAAGAACATCGACGGCTACTTCAAGATCTACAGCAAGCACACCCCCATCATCGT
GGAGCCTGAGAGAGACCTGCCCCAGGGCTTCAGCGCCCTGGAGCCCCTGGTGGACC
TGCCCATCGGCATCAACATCACCAGATTCCAGACCCTGCTGGCCCTGCACAGAAGCT
ACCTGACCCCCGGCGACAGCAGCAGCGGCTGGACCGCCGGCGCCGCCGCCTACTAC
GTGGGCTACCTGCAGCCCAGAACCTTCCTGCTGAAGTACAACGAGAACGGCACCAT
CACCGACGCCGTGGACTGCGCCCTGGACCCCCTGAGCGAGACCAAGTGCACCCTGA
AGAGCTTCACCGTGGAGAAGGGCATCTACCAGACCAGCAACTTCAGAGTGCAGCCC
ACCGAGAGCATCGTGAGATTCCCCAACATCACCAACCTGTGCCCCTTCGACGAGGTG
TTCAACGCCACCAGATTCGCCAGCGTGTACGCCTGGAACAGAAAGAGAATCAGCAA
CTGCGTGGCCGACTACAGCGTGCTGTACAACCTGGCCCCTTTCTTCACCTTCAAGTG
CTACGGCGTGAGCCCCACCAAGCTGAACGACCTGTGCTTCACCAACGTGTACGCCG
ACAGCTTCGTGATCAGAGGCGACGAGGTGAGACAGATCGCCCCCGGCCAGACCGGC
AACATCGCCGACTACAACTACAAGCTGCCCGACGACTTCACCGGCTGCGTGATCGCC
TGGAACAGCAACAAGCTGGACAGCAAGGTGAGCGGCAACTACAACTACCTGTACAG
ACTGTTCAGAAAGAGCAACCTGAAGCCCTTCGAGAGAGACATCAGCACCGAGATCT
ACCAGGCCGGCAACAAGCCCTGCAACGGCGTGGCCGGCTTCAACTGCTACTTCCCCC
TGAAGAGCTACAGCTTCCGGCCCACCTACGGCGTGGGCCACCAGCCCTACAGAGTG
GTGGTGCTGAGCTTCGAGCTGCTGCACGCCCCCGCCACCGTGTGCGGCCCCAAGAA
GAGCACCAACCTGGTGAAGAACAAGTGCGTGAACTTCAACTTCAACGGCCTGAAGG
GCACCGGCGTGCTGACCGAGAGCAACAAGAAGTTCCTGCCCTTCCAGCAGTTCGGC
AGAGACATCGCCGACACCACCGACGCCGTGAGAGACCCCCAGACCCTGGAGATCCT
GGACATCACCCCCTGCAGCTTCGGCGGCGTGAGCGTGATCACCCCCGGCACCAACA
CCAGCAACCAGGTGGCCGTGCTGTACCAGGGCGTGAACTGCACCGAGGTGCCCGTG
GCCATCCACGCCGACCAGCTGACCCCCACCTGGAGAGTGTACAGCACCGGCAGCAA
CGTGTTCCAGACCAGAGCCGGCTGCCTGATCGGCGCCGAGTACGTGAACAACAGCT
ACGAGTGCGACATCCCCATCGGCGCCGGCATCTGCGCCAGCTACCAGACCCAGACC
AAGAGCCACGGAAGCGCCAGCAGCGTGGCCAGCCAGAGCATCATCGCCTACACCAT
GAGCCTGGGCGCCGAGAACAGCGTGGCCTACAGCAACAACAGCATCGCCATCCCCA
CCAACTTCACCATCAGCGTGACCACCGAGATCCTGCCCGTGAGCATGACCAAGACC
AGCGTGGACTGCACCATGTACATCTGCGGCGACAGCACCGAGTGCAGCAACCTGCT
GCTGCAGTACGGCAGCTTCTGCACCCAGCTGAAGAGAGCCCTGACCGGCATCGCCG
TGGAGCAGGACAAGAACACCCAGGAGGTGTTCGCCCAGGTGAAGCAGATCTACAAG
ACCCCCCCCATCAAGTACTTCGGCGGCTTCAACTTCAGCCAGATCCTGCCCGACCCC
AGCAAGCCCAGCAAGAGAAGCTTCATCGAGGACCTGCTGTTCAACAAGGTGACCCT
GGCCGACGCCGGCTTCATCAAGCAGTACGGCGACTGCCTGGGCGACATCGCCGCCA
GAGACCTGATCTGCGCCCAGAAGTTCAAGGGCCTGACCGTGCTGCCCCCCCTGCTGA
CCGACGAGATGATCGCCCAGTACACCAGCGCCCTGCTGGCCGGCACCATCACCAGC
GGCTGGACCTTCGGCGCCGGCGCCGCCCTGCAGATCCCCTTCGCCATGCAGATGGCC
TACAGATTCAACGGCATCGGCGTGACCCAGAACGTGCTGTACGAGAACCAGAAGCT
GATCGCCAACCAGTTCAACAGCGCCATCGGCAAGATCCAGGACAGCCTGAGCAGCA
CCGCCAGCGCCCTGGGCAAGCTGCAGGACGTGGTGAACCACAACGCCCAGGCCCTG
AACACCCTGGTGAAGCAGCTGAGCAGCAAGTTCGGCGCCATCAGCAGCGTGCTGAA
CGATATCTTCAGCAGACTGGACCCACCTGAGGCCGAGGTGCAGATCGACAGACTGA
TCACCGGCAGACTGCAGAGCCTGCAGACCTACGTGACCCAGCAGCTGATCAGAGCC
GCCGAGATCAGAGCCAGCGCCAACCTGGCCGCCACCAAGATGAGCGAGTGCGTGCT
GGGCCAGAGCAAGAGAGTGGACTTCTGCGGCAAGGGCTACCACCTGATGAGCTTCC
CCCAGAGCGCCCCCCACGGCGTGGTGTTCCTGCACGTGACCTACGTGCCCGCCCAGG
AGAAGAACTTCACCACCGCCCCCGCCATCTGCCACGACGGCAAGGCCCACTTCCCC
AGAGAGGGCGTGTTCGTGAGCAACGGCACCCACTGGTTCGTGACCCAGAGAAACTT
CTACGAGCCCCAGATCATCACCACCGACAACACCTTCGTGAGCGGCAACTGCGACG
TGGTGATCGGCATCGTGAACAACACCGTGTACGACCCCCTGCAGCCCGAGCTGGAC
AGCTTCAAGGAGGAGCTGGACAAGTACTTCAAGAACCACACCAGCCCCGACGTGGA
CCTGGGCGACATCAGCGGCATCAACGCCAGCGTGGTGAACATCCAGAAGGAGATCG
ACAGACTGAACGAGGTGGCCAAGAATTTAAATGAGAGCCTGATCGACCTGCAGGAG
CTGGGCAAGTACGAGCAGTACATCAAGTGGCCCTGGTACATCTGGCTGGGCTTCATC
GCCGGCCTGATCGCCATCGTGATGGTGACCATCATGCTGTGCTGCATGACCAGCTGC
TGCAGCTGCCTGAAGGGCTGCTGCAGCTGCGGCAGCTGCTGCAAGTTCGACGAGGA
CGACAGCGAGCCCGTGCTGAAGGGCGTGAAGCTGCACTACACCTAA
SEQ ID NO: 28-CorovaxG.3-O.FR-amino acid sequence
MFVFLVLLPLVSSQCVNLTTRTQLPPAYTNSFTRGVYYPDKVFRSSVLHSTQDLFLPFFS
NVTWFHVISGTNGTKRFDNPVLPFNDGVYFASIEKSNIIRGWIFGTTLDSKTQSLLIVNNA
TNVVIKVCEFQFCNDPFLDHKNNKSWMESEFRVYSSANNCTFEYVSQPFLMDLEGKQG
NFKNLREFVFKNIDGYFKIYSKHTPIIVEPERDLPQGFSALEPLVDLPIGINITRFQTLLALH
RSYLTPGDSSSGWTAGAAAYYVGYLQPRTFLLKYNENGTITDAVDCALDPLSETKCTLK
SFTVEKGIYQTSNFRVQPTESIVRFPNITNLCPFDEVENATRFASVYAWNRKRISNCVAD
YSVLYNLAPFFTFKCYGVSPTKLNDLCFTNVYADSFVIRGDEVRQIAPGQTGNIADYNY
KLPDDFTGCVIAWNSNKLDSKVSGNYNYLYRLFRKSNLKPFERDISTEIYQAGNKPCNG
VAGENCYFPLKSYSFRPTYGVGHQPYRVVVLSFELLHAPATVCGPKKSTNLVKNKCVN
FNFNGLKGTGVLTESNKKFLPFQQFGRDIADTTDAVRDPQTLEILDITPCSFGGVSVITPG
TNTSNQVAVLYQGVNCTEVPVAIHADQLTPTWRVYSTGSNVFQTRAGCLIGAEYVNNS
YECDIPIGAGICASYQTQTKSHGSASSVASQSIIAYTMSLGAENSVAYSNNSIAIPTNFTIS
VTTEILPVSMTKTSVDCTMYICGDSTECSNLLLQYGSFCTQLKRALTGIAVEQDKNTQEV
FAQVKQIYKTPPIKYFGGFNFSQILPDPSKPSKRSFIEDLLFNKVTLADAGFIKQYGDCLG
DIAARDLICAQKFKGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNHNAQALNT
LVKQLSSKFGAISSVLNDIFSRLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCSCGSCCKFDEDD
SEPVLKGVKLHYT

### REFERENCES

All references, including publications, patent applications, and patents, cited herein are hereby incorporated by reference to the same extent as if each reference were individually and specifically indicated to be incorporated by reference and were set forth in its entirety herein.
Abbink, P., A. A. Lemckert, B. A. Ewald, D. M. Lynch, M. Denholtz, S. Smits, L. Holterman, I. Damen, R. Vogels, A. R. Thorner, K. L. O'Brien, A. Carville, K. G. Mansfield, J. Goudsmit, M. J. Havenga and D. H. Barouch (2007). “Comparative seroprevalence and immunogenicity of six rare serotype recombinant adenovirus vaccine vectors from subgroups B and D.” J Virol 81(9): 4654-4663.
Bauvois, B., J. Nguyen, R. Tang, C. Billard and J. P. Kolb (2009). “Types I and II interferons upregulate the costimulatory CD80 molecule in monocytes via interferon regulatory factor-1.” Biochem Pharmacol 78(5): 514-522.
Berges, C., C. Naujokat, S. Tinapp, H. Wieczorek, A. Hoh, M. Sadeghi, G. Opelz and V. Daniel (2005). “A cell line model for the differentiation of human dendritic cells.” Biochem Biophys Res Commun 333(3): 896-907.
Binder, A. M., H. M. Biggs, A. K. Haynes, C. Chommanard, X. Lu, D. D. Erdman, J. T. Watson and S. I. Gerber (2017). “Human Adenovirus Surveillance—United States, 2003-2016.” MMWR Morb Mortal Wkly Rep 66(39): 1039-1042.
Bliss, C. M., A. J. Parsons, R. Nachbagauer, J. R. Hamilton, F. Cappuccini, M. Ulaszewska, J. P. Webber, A. Clayton, A. V. S. Hill and L. Coughlan (2020). “Targeting Antigen to the Surface of EVs Improves the In Vivo Immunogenicity of Human and Non-human Adenoviral Vaccines in Mice.” Mol Ther Methods Clin Dev 16: 108-125.
Bloom, E. T., H. S. Mostowski and J. A. Horvath (1994). “Does the age-related change in CD44-defined T-cell subsets have functional significance for cytotoxic T lymphocyte generation?” Immunol Lett 40(3): 251-258.
Bos, R., L. Rutten, J. E. M. van der Lubbe, M. J. G. Bakkers, G. Hardenberg, F. Wegmann, D. Zuijdgeest, A. H. de Wilde, A. Koornneef, A. Verwilligen, D. van Manen, T. Kwaks, R. Vogels, T. J. Dalebout, S. K. Myeni, M. Kikkert, E. J. Snijder, Z. Li, D. H. Barouch, J. Vellinga, J. P. M. Langedijk, R. C. Zahn, J. Custers and H. Schuitemaker (2020). “Ad26 vector-based COVID-19 vaccine encoding a prefusion-stabilized SARS-CoV-2 Spike immunogen induces potent humoral and cellular immune responses.” NPJ Vaccines 5: 91.
Bouazzaoui, A., A. A. H. Abdellatif, F. A. Al-Allaf, N. M. Bogari, S. Al-Dehlawi and S. H. Qari (2021). “Strategies for Vaccination: Conventional Vaccine Approaches Versus New-Generation Strategies in Combination with Adjuvants.” Pharmaceutics 13(2).
Bournazos, S., A. Gupta and J. V. Ravetch (2020). “The role of IgG Fc receptors in antibody-dependent enhancement.” Nat Rev Immunol 20(10): 633-643.
Brooks, A. R., R. N. Harkins, P. Wang, H. S. Qian, P. Liu and G. M. Rubanyi (2004). “Transcriptional silencing is associated with extensive methylation of the CMV promoter following adenoviral gene delivery to muscle.” J Gene Med 6(4): 395-404.
Corbett, K. S., D. K. Edwards, S. R. Leist, O. M. Abiona, S. Boyoglu-Barnum, R. A. Gillespie, S. Himansu, A. Schafer, C. T. Ziwawo, A. T. DiPiazza, K. H. Dinnon, S. M. Elbashir, C. A. Shaw, A. Woods, E. J. Fritch, D. R. Martinez, K. W. Bock, M. Minai, B. M. Nagata, G. B. Hutchinson, K. Wu, C. Henry, K. Bahl, D. Garcia-Dominguez, L. Ma, I. Renzi, W. P. Kong, S. D. Schmidt, L. Wang, Y. Zhang, E. Phung, L. A. Chang, R. J. Loomis, N. E. Altaras, E. Narayanan, M. Metkar, V. Presnyak, C. Liu, M. K. Louder, W. Shi, K. Leung, E. S. Yang, A. West, K. L. Gully, L. J. Stevens, N. Wang, D. Wrapp, N. A. Doria-Rose, G. Stewart-Jones, H. Bennett, G. S. Alvarado, M. C. Nason, T. J. Ruckwardt, J. S. McLellan, M. R. Denison, J. D. Chappell, I. N. Moore, K. M. Morabito, J. R. Mascola, R. S. Baric, A. Carfi and B. S. Graham (2020). “SARS-CoV-2 mRNA vaccine design enabled by prototype pathogen preparedness.” Nature 586(7830): 567-571.
Coughlan, L. (2020). “Factors Which Contribute to the Immunogenicity of Non-replicating Adenoviral Vectored Vaccines.” Front Immunol 11: 909.
Cox, R. J. and K. A. Brokstad (2020). “Not just antibodies: B cells and T cells mediate immunity to COVID-19.” Nat Rev Immunol 20(10): 581-582.
Crawford, K. H. D., R. Eguia, A. S. Dingens, A. N. Loes, K. D. Malone, C. R. Wolf, H. Y. Chu, M. A. Tortorici, D. Veesler, M. Murphy, D. Pettie, N. P. King, A. B. Balazs and J. D. Bloom (2020). “Protocol and Reagents for Pseudotyping Lentiviral Particles with SARS-CoV-2 Spike Protein for Neutralization Assays.” Viruses 12(5).
Chakraborty, S., V. Mallajosyula, C. M. Tato, G. S. Tan and T. T. Wang (2021). “SARS-CoV-2 vaccines in advanced clinical trials: Where do we stand?” Advanced drug delivery reviews 172: 314-338.
Chen, W. Y., E. C. Bailey, S. L. McCune, J. Y. Dong and T. M. Townes (1997). “Reactivation of silenced, virally transduced genes by inhibitors of histone deacetylase.” Proc Natl Acad Sci USA 94(11): 5798-5803.
Choi, K. H., H. Basma, J. Singh and P. W. Cheng (2005). “Activation of CMV promoter-controlled glycosyltransferase and beta-galactosidase glycogenes by butyrate, tricostatin A, and 5-aza-2′-deoxycytidine.” Glycoconj J 22(1-2): 63-69.
Chondronasiou, D., T. Eisden, A. G. M. Stam, Q. L. Matthews, M. Icyuz, E. Hooijberg, I. Dmitriev, D. T. Curiel, T. D. de Gruijl and R. van de Ven (2018). “Improved Induction of Anti-Melanoma T Cells by Adenovirus-5/3 Fiber Modification to Target Human DCs.” Vaccines (Basel) 6(3).
Dan, J. M., J. Mateus, Y. Kato, K. M. Hastie, E. D. Yu, C. E. Faliti, A. Grifoni, S. I. Ramirez, S. Haupt, A. Frazier, C. Nakao, V. Rayaprolu, S. A. Rawlings, B. Peters, F. Krammer, V. Simon, E. O. Saphire, D. M. Smith, D. Weiskopf, A. Sette and S. Crotty (2021). “Immunological memory to SARS-CoV-2 assessed for up to 8 months after infection.” Science 371(6529).
Dicks, M. D., A. J. Spencer, L. Coughlan, K. Bauza, S. C. Gilbert, A. V. Hill and M. G. Cottingham (2015). “Differential immunogenicity between HAdV-5 and chimpanzee adenovirus vector ChAdOx1 is independent of fiber and penton RGD loop sequences in mice.” Sci Rep 5: 16756.
Donello, J. E., J. E. Loeb and T. J. Hope (1998). “Woodchuck hepatitis virus contains a tripartite posttranscriptional regulatory element.” J Virol 72(6): 5085-5092.
Fidler, D. P. (2010). “Negotiating equitable access to influenza vaccines: global health diplomacy and the controversies surrounding avian influenza H5N1 and pandemic influenza H1N1.” PLoS medicine 7(5): e1000247-e1000247.
Gonzalez-Candelas, F., M. A. Shaw, T. Phan, U. Kulkarni-Kale, D. Paraskevis, F. Luciani, H. Kimura and M. Sironi (2021). “One year into the pandemic: Short-term evolution of SARS-CoV-2 and emergence of new lineages.” Infect Genet Evol 92: 104869.
Graham, B. S. (2020). “Rapid COVID-19 vaccine development.” Science 368(6494): 945-946.
Gray, S. J., S. B. Foti, J. W. Schwartz, L. Bachaboina, B. Taylor-Blake, J. Coleman, M. D. Ehlers, M. J. Zylka, T. J. McCown and R. J. Samulski (2011). “Optimizing promoters for recombinant adeno-associated virus-mediated gene expression in the peripheral and central nervous system using self-complementary vectors.” Hum Gene Ther 22(9): 1143-1153.
Guse, K., M. Suzuki, G. Sule, T. K. Bertin, H. Tyynismaa, S. Ahola-Erkkila, D. Palmer, A. Suomalainen, P. Ng, V. Cerullo, A. Hemminki and B. Lee (2012). “Capsid-modified adenoviral vectors for improved muscle-directed gene therapy.” Hum Gene Ther 23(10): 1065-1070.
Guse, K., M. Suzuki, G. Sule, T. K. Bertin, H. Tyynismaa, S. Ahola-Erkkila, D. Palmer, A. Suomalainen, P. Ng, V. Cerullo, A. Hemminki and B. Lee (2012). “Capsid-modified adenoviral vectors for improved muscle-directed gene therapy.” Human gene therapy 23(10): 1065-1070.
Hensley, S. E., A. S. Cun, W. Giles-Davis, Y. Li, Z. Xiang, M. O. Lasaro, B. R. Williams, R. H. Silverman and H. C. Ertl (2007). “Type I interferon inhibits antibody responses induced by a chimpanzee adenovirus vector.” Mol Ther 15(2): 393-403.
Iwasaki, A. and Y. Yang (2020). “The potential danger of suboptimal antibody responses in COVID-19.” Nat Rev Immunol 20(6): 339-341.
Kak, G., M. Raza and B. K. Tiwari (2018). “Interferon-gamma (IFN-gamma): Exploring its implications in infectious diseases.” Biomol Concepts 9(1): 64-79.
Korber, B., W. M. Fischer, S. Gnanakaran, H. Yoon, J. Theiler, W. Abfalterer, N. Hengartner, E. E. Giorgi, T. Bhattacharya, B. Foley, K. M. Hastie, M. D. Parker, D. G. Partridge, C. M. Evans, T. M. Freeman, T. I. de Silva, C.-G. G. Sheffield, C. McDanal, L. G. Perez, H. Tang, A. Moon-Walker, S. P. Whelan, C. C. LaBranche, E. O. Saphire and D. C. Montefiori (2020). “Tracking Changes in SARS-CoV-2 Spike: Evidence that D614G Increases Infectivity of the COVID-19 Virus.” Cell 182(4): 812-827 e819.
Kyriakidis, N. C., A. López-Cortés, E. V. Gonzalez, A. B. Grimaldos and E. O. Prado (2021). “SARS-CoV-2 vaccines strategies: a comprehensive review of phase 3 candidates.” npj Vaccines 6(1): 28.
Le Bert, N., H. E. Clapham, A. T. Tan, W. N. Chia, C. Y. L. Tham, J. M. Lim, K. Kunasegaran, L. W. L. Tan, C. A. Dutertre, N. Shankar, J. M. E. Lim, L. J. Sun, M. Zahari, Z. M. Tun, V. Kumar, B. L. Lim, S. H. Lim, A. Chia, Y. J. Tan, P. A. Tambyah, S. Kalimuddin, D. Lye, J. G. H. Low, L. F. Wang, W. Y. Wan, L. Y. Hsu, A. Bertoletti and C. C. Tam (2021). “Highly functional virus-specific cellular immune response in asymptomatic SARS-CoV-2 infection.” J Exp Med 218(5).
Lefrancois, L. and D. Masopust (2002). “T cell immunity in lymphoid and non-lymphoid tissues.” Curr Opin Immunol 14(4): 503-508.
Liao, M., Y. Liu, J. Yuan, Y. Wen, G. Xu, J. Zhao, L. Cheng, J. Li, X. Wang, F. Wang, L. Liu, I. Amit, S. Zhang and Z. Zhang (2020). “Single-cell landscape of bronchoalveolar immune cells in patients with COVID-19.” Nat Med 26(6): 842-844.
Lien, C. E., Y. J. Lin, C. Chen, W. C. Lian, T. Y. Kuo, J. D. Campbell, P. Traquina, M. Y. Lin, L. T. Liu, Y. S. Chuang, H. Y. Ko, C. C. Liao, Y. H. Chen, J. T. Jan, H. H. Ma, C. P. Sun, Y. S. Lin, P. Y. Wu, Y. C. Wang, M. H. Tao and Y. L. Lin (2021). “CpG-adjuvanted stable prefusion SARS-CoV-2 spike protein protected hamsters from SARS-CoV-2 challenge.” Sci Rep 11(1): 8761.
Logunov, D. Y., I. V. Dolzhikova, O. V. Zubkova, A. I. Tukhvatulin, D. V. Shcheblyakov, A. S. Dzharullaeva, D. M. Grousova, A. S. Erokhova, A. V. Kovyrshina, A. G. Botikov, F. M. Izhaeva, O. Popova, T. A. Ozharovskaya, I. B. Esmagambetov, I. A. Favorskaya, D. I. Zrelkin, D. V. Voronina, D. N. Shcherbinin, A. S. Semikhin, Y. V. Simakova, E. A. Tokarskaya, N. L. Lubenets, D. A. Egorova, M. M. Shmarov, N. A. Nikitenko, L. F. Morozova, E. A. Smolyarchuk, E. V. Kryukov, V. F. Babira, S. V. Borisevich, B. S. Naroditsky and A. L. Gintsburg (2020). “Safety and immunogenicity of an rAd26 and rAd5 vector-based heterologous prime-boost COVID-19 vaccine in two formulations: two open, non-randomised phase 1/2 studies from Russia.” Lancet 396(10255): 887-897.
Lopez, M. V., A. A. Rivera, D. L. Viale, L. Benedetti, N. Cuneo, K. J. Kimball, M. Wang, J. T. Douglas, Z. B. Zhu, A. I. Bravo, M. Gidekel, R. D. Alvarez, D. T. Curiel and O. L. Podhajcer (2012). “A Tumor-stroma Targeted Oncolytic Adenovirus Replicated in Human Ovary Cancer Samples and Inhibited Growth of Disseminated Solid Tumors in Mice.” Mol Ther.
Ma, X., J. M. Chow, G. Gri, G. Carra, F. Gerosa, S. F. Wolf, R. Dzialo and G. Trinchieri (1996). “The interleukin 12 p40 gene promoter is primed by interferon gamma in monocytic cells.” J Exp Med 183(1): 147-157.
McMahan, K., J. Yu, N. B. Mercado, C. Loos, L. H. Tostanoski, A. Chandrashekar, J. Liu, L. Peter, C. Atyeo, A. Zhu, E. A. Bondzie, G. Dagotto, M. S. Gebre, C. Jacob-Dolan, Z. Li, F. Nampanya, S. Patel, L. Pessaint, A. Van Ry, K. Blade, J. Yalley-Ogunro, M. Cabus, R. Brown, A. Cook, E. Teow, H. Andersen, M. G. Lewis, D. A. Lauffenburger, G. Alter and D. H. Barouch (2021). “Correlates of protection against SARS-CoV-2 in rhesus macaques.” Nature 590(7847): 630-634.
Mennechet, F. J. D., O. Paris, A. R. Ouoba, S. Salazar Arenas, S. B. Sirima, G. R. Takoudjou Dzomo, A. Diarra, I. T. Traore, D. Kania, K. Eichholz, E. A. Weaver, E. Tuaillon and E. J. Kremer (2019). “A review of 65 years of human adenovirus seroprevalence.” Expert Rev Vaccines 18(6): 597-613.
Mercado, N. B., R. Zahn, F. Wegmann, C. Loos, A. Chandrashekar, J. Yu, J. Liu, L. Peter, K. McMahan, L. H. Tostanoski, X. He, D. R. Martinez, L. Rutten, R. Bos, D. van Manen, J. Vellinga, J. Custers, J. P. Langedijk, T. Kwaks, M. J. G. Bakkers, D. Zuijdgeest, S. K. Rosendahl Huber, C. Atyeo, S. Fischinger, J. S. Burke, J. Feldman, B. M. Hauser, T. M. Caradonna, E. A. Bondzie, G. Dagotto, M. S. Gebre, E. Hoffman, C. Jacob-Dolan, M. Kirilova, Z. Li, Z. Lin, S. H. Mahrokhian, L. F. Maxfield, F. Nampanya, R. Nityanandam, J. P. Nkolola, S. Patel, J. D. Ventura, K. Verrington, H. Wan, L. Pessaint, A. Van Ry, K. Blade, A. Strasbaugh, M. Cabus, R. Brown, A. Cook, S. Zouantchangadou, E. Teow, H. Andersen, M. G. Lewis, Y. Cai, B. Chen, A. G. Schmidt, R. K. Reeves, R. S. Baric, D. A. Lauffenburger, G. Alter, P. Stoffels, M. Mammen, J. Van Hoof, H. Schuitemaker and D. H. Barouch (2020). “Single-shot Ad26 vaccine protects against SARS-CoV-2 in rhesus macaques.” Nature 586(7830): 583-588.
Mercier, S., S. Verhaagh, J. Goudsmit, A. Lemckert, M. Monteil, M. Havenga and M. Eloit (2004). “Adenovirus fibre exchange alters cell tropism in vitro but not transgene-specific T CD8+ immune responses in vivo.” J Gen Virol 85(Pt 5): 1227-1236.
Nie, J., Q. Li, J. Wu, C. Zhao, H. Hao, H. Liu, L. Zhang, L. Nie, H. Qin, M. Wang, Q. Lu, X. Li, Q. Sun, J. Liu, C. Fan, W. Huang, M. Xu and Y. Wang (2020). “Quantification of SARS-CoV-2 neutralizing antibody by a pseudotyped virus-based assay.” Nat Protoc.
Okada, P., R. Buathong, S. Phuygun, T. Thanadachakul, S. Parnmen, W. Wongboot, S. Waicharoen, S. Wacharapluesadee, S. Uttayamakul, A. Vachiraphan, M. Chittaganpitch, N. Mekha, N. Janejai, S. Iamsirithaworn, R. T. Lee and S. Maurer-Stroh (2020). “Early transmission patterns of coronavirus disease 2019 (COVID-19) in travellers from Wuhan to Thailand, January 2020.” Eurosurveillance 25(8): 2000097.
Quinn, K. M., A. Da Costa, A. Yamamoto, D. Berry, R. W. Lindsay, P. A. Darrah, L. Wang, C. Cheng, W. P. Kong, J. G. Gall, A. Nicosia, A. Folgori, S. Colloca, R. Cortese, E. Gostick, D. A. Price, C. E. Gomez, M. Esteban, L. S. Wyatt, B. Moss, C. Morgan, M. Roederer, R. T. Bailer, G. J. Nabel, R. A. Koup and R. A. Seder (2013). “Comparative analysis of the magnitude, quality, phenotype, and protective capacity of simian immunodeficiency virus gag-specific CD8+ T cells following human-, simian-, and chimpanzee-derived recombinant adenoviral vector immunization.” J Immunol 190(6): 2720-2735.
Quinn, K. M., D. E. Zak, A. Costa, A. Yamamoto, K. Kastenmuller, B. J. Hill, G. M. Lynn, P. A. Darrah, R. W. Lindsay, L. Wang, C. Cheng, A. Nicosia, A. Folgori, S. Colloca, R. Cortese, E. Gostick, D. A. Price, J. G. Gall, M. Roederer, A. Aderem and R. A. Seder (2015). “Antigen expression determines adenoviral vaccine potency independent of IFN and STING signaling.” J Clin Invest 125(3): 1129-1146.
Ramasamy, M. N., A. M. Minassian, K. J. Ewer, A. L. Flaxman, P. M. Folegatti, D. R. Owens, M. Voysey, P. K. Aley, B. Angus, G. Babbage, S. Belij-Rammerstorfer, L. Berry, S. Bibi, M. Bittaye, K. Cathie, H. Chappell, S. Charlton, P. Cicconi, E. A. Clutterbuck, R. Colin-Jones, C. Dold, K. R. W. Emary, S. Fedosyuk, M. Fuskova, D. Gbesemete, C. Green, B. Hallis, M. M. Hou, D. Jenkin, C. C. D. Joe, E. J. Kelly, S. Kerridge, A. M. Lawrie, A. Lelliott, M. N. Lwin, R. Makinson, N. G. Marchevsky, Y. Mujadidi, A. P. S. Munro, M. Pacurar, E. Plested, J. Rand, T. Rawlinson, S. Rhead, H. Robinson, A. J. Ritchie, A. L. Ross-Russell, S. Saich, N. Singh, C. C. Smith, M. D. Snape, R. Song, R. Tarrant, Y. Themistocleous, K. M. Thomas, T. L. Villafana, S. C. Warren, M. E. E. Watson, A. D. Douglas, A. V. S. Hill, T. Lambe, S. C. Gilbert, S. N. Faust, A. J. Pollard and C. V. T. G. Oxford (2021). “Safety and immunogenicity of ChAdOx1 nCoV-19 vaccine administered in a prime-boost regimen in young and old adults (COV002): a single-blind, randomised, controlled, phase 2/3 trial.” Lancet 396(10267): 1979-1993.
Rawat, K., P. Kumari and L. Saha (2021). “COVID-19 vaccine: A recent update in pipeline vaccines, their design and development strategies.” European journal of pharmacology 892: 173751-173751.
Remakus, S. and L. J. Sigal (2013). “Memory CD8(+) T cell protection.” Adv Exp Med Biol 785: 77-86.
Roberts, A. D., K. H. Ely and D. L. Woodland (2005). “Differential contributions of central and effector memory T cells to recall responses.” J Exp Med 202(1): 123-133.
Rydyznski Moderbacher, C., S. I. Ramirez, J. M. Dan, A. Grifoni, K. M. Hastie, D. Weiskopf, S. Belanger, R. K. Abbott, C. Kim, J. Choi, Y. Kato, E. G. Crotty, C. Kim, S. A. Rawlings, J. Mateus, L. P. V. Tse, A. Frazier, R. Baric, B. Peters, J. Greenbaum, E. Ollmann Saphire, D. M. Smith, A. Sette and S. Crotty (2020). “Antigen-Specific Adaptive Immunity to SARS-CoV-2 in Acute COVID-19 and Associations with Age and Disease Severity.” Cell 183(4): 996-1012 e1019.
Sadoff, J., M. Le Gars, G. Shukarev, D. Heerwegh, C. Truyers, A. M. de Groot, J. Stoop, S. Tete, W. Van Damme, I. Leroux-Roels, P.-J. Berghmans, M. Kimmel, P. Van Damme, J. de Hoon, W. Smith, K. E. Stephenson, S. C. De Rosa, K. W. Cohen, M. J. McElrath, E. Cormier, G. Scheper, D. H. Barouch, J. Hendriks, F. Struyf, M. Douoguih, J. Van Hoof and H. Schuitemaker (2021). “Interim Results of a Phase 1-2a Trial of Ad26.COV2.S Covid-19 Vaccine.” New England Journal of Medicine.
Sadoff, J., M. Le Gars, G. Shukarev, D. Heerwegh, C. Truyers, A. M. de Groot, J. Stoop, S. Tete, W. Van Damme, I. Leroux-Roels, P. J. Berghmans, M. Kimmel, P. Van Damme, J. de Hoon, W. Smith, K. E. Stephenson, S. C. De Rosa, K. W. Cohen, M. J. McElrath, E. Cormier, G. Scheper, D. H. Barouch, J. Hendriks, F. Struyf, M. Douoguih, J. Van Hoof and H. Schuitemaker (2021). “Interim Results of a Phase 1-2a Trial of Ad26.COV2.S Covid-19 Vaccine.” N Engl J Med 384(19): 1824-1835.
Sallusto, F., A. Lanzavecchia, K. Araki and R. Ahmed (2010). “From vaccines to memory and back.” Immunity 33(4): 451-463.
Sallusto, F., D. Lenig, R. Forster, M. Lipp and A. Lanzavecchia (1999). “Two subsets of memory T lymphocytes with distinct homing potentials and effector functions.” Nature 401(6754): 708-712.
Sayedahmed, E. E., R. Kumari, S. Shukla, A. O. Hassan, S. I. Mohammed, I. A. York, S. Gangappa, S. Sambhara and S. K. Mittal (2018). “Longevity of adenovirus vector immunity in mice and its implications for vaccine efficacy.” Vaccine 36(45): 6744-6751.
Schmidt, M. E. and S. M. Varga (2018). “The CD8 T Cell Response to Respiratory Virus Infections.” Front Immunol 9: 678.
Schroder, K., P. J. Hertzog, T. Ravasi and D. A. Hume (2004). “Interferon-gamma: an overview of signals, mechanisms and functions.” J Leukoc Biol 75(2): 163-189.
Sester, M., U. Sester, S. Alarcon Salvador, G. Heine, S. Lipfert, M. Girndt, B. Gartner and H. Kohler (2002). “Age-related decrease in adenovirus-specific T cell responses.” J Infect Dis 185(10): 1379-1387.
Shibaki, A. and S. I. Katz (2002). “Induction of skewed Th1/Th2 T-cell differentiation via subcutaneous immunization with Freund's adjuvant.” Exp Dermatol 11(2): 126-134.
Sun, J., R. Madan, C. L. Karp and T. J. Braciale (2009). “Effector T cells control lung inflammation during acute influenza virus infection by producing IL-10.” Nat Med 15(3): 277-284.
Tan, W. G., H. T. Jin, E. E. West, P. Penaloza-MacMaster, A. Wieland, M. J. Zilliox, M. J. McElrath, D. H. Barouch and R. Ahmed (2013). “Comparative analysis of simian immunodeficiency virus gag-specific effector and memory CD8+ T cells induced by different adenovirus vectors.” J Virol 87(3): 1359-1372.
Tregoning, J. S., E. S. Brown, H. M. Cheeseman, K. E. Flight, S. L. Higham, N. M. Lemm, B. F. Pierce, D. C. Stirling, Z. Wang and K. M. Pollock (2020). “Vaccines for COVID-19.” Clinical and experimental immunology 202(2): 162-192.
Trinh, H. V., G. Lesage, V. Chennamparampil, B. Vollenweider, C. J. Burckhardt, S. Schauer, M. Havenga., U. F. Greber and S. Hemmi (2012). “Avidity binding of human adenovirus serotypes 3 and 7 to the membrane cofactor CD46 triggers infection.” Journal of virology 86(3): 1623-1637.
van de Berg, D., Z. Kis, C. F. Behmer, K. Samnuan, A. K. Blakney, C. Kontoravdi, R. Shattock and N. Shah (2021). “Quality by design modelling to support rapid RNA vaccine production against emerging infectious diseases.” npj Vaccines 6(1): 65.
van de Ven, R., J. J. Lindenberg, D. Oosterhoff, M. P. van den Tol, R. A. Rosalia, M. Murakami, M. Everts, G. L. Scheffer, R. J. Scheper, T. D. de Gruijl and D. T. Curiel (2009). “Selective transduction of mature DC in human skin and lymph nodes by CD80/CD86-targeted fiber-modified adenovirus-5/3.” J Immunother 32(9): 895-906.
van Doremalen, N., T. Lambe, A. Spencer, S. Belij-Rammerstorfer, J. N. Purushotham, J. R. Port, V. A. Avanzato, T. Bushmaker, A. Flaxman, M. Ulaszewska, F. Feldmann, E. R. Allen, H. Sharpe, J. Schulz, M. Holbrook, A. Okumura, K. Meade-White, L. Perez-Perez, N. J. Edwards, D. Wright, C. Bissett, C. Gilbride, B. N. Williamson, R. Rosenke, D. Long, A. Ishwarbhai, R. Kailath, L. Rose, S. Morris, C. Powers, J. Lovaglio, P. W. Hanley, D. Scott, G. Saturday, E. de Wit, S. C. Gilbert and V. J. Munster (2020). “ChAdOx1 nCoV-19 vaccine prevents SARS-CoV-2 pneumonia in rhesus macaques.” Nature 586(7830): 578-582.
Viale, D. L., E. G. Cafferata, D. Gould, C. Rotondaro, Y. Chemajovsky, D. T. Curiel, O. L. Podhajcer and M. V. Lopez (2013). “Therapeutic improvement of a stroma-targeted CRAd by incorporating motives responsive to the melanoma microenvironment.” J Invest Dermatol 133(11): 2576-2584.
Voysey, M., S. A. C. Clemens, S. A. Madhi, L. Y. Weckx, P. M. Folegatti, P. K. Aley, B. Angus, V. L. Baillie, S. L. Barnabas, Q. E. Bhorat, S. Bibi, C. Briner, P. Cicconi, A. M. Collins, R. Colin-Jones, C. L. Cutland, T. C. Darton, K. Dheda, C. J. A. Duncan, K. R. W. Emary, K. J. Ewer, L. Fairlie, S. N. Faust, S. Feng, D. M. Ferreira, A. Finn, A. L. Goodman, C. M. Green, C. A. Green, P. T. Heath, C. Hill, H. Hill, I. Hirsch, S. H. C. Hodgson, A. Izu, S. Jackson, D. Jenkin, C. C. D. Joe, S. Kerridge, A. Koen, G. Kwatra, R. Lazarus, A. M. Lawrie, A. Lelliott, V. Libri, P. J. Lillie, R. Mallory, A. V. A. Mendes, E. P. Milan, A. M. Minassian, A. McGregor, H. Morrison, Y. F. Mujadidi, A. Nana, P. J. O'Reilly, S. D. Padayachee, A. Pittella, E. Plested, K. M. Pollock, M. N. Ramasamy, S. Rhead, A. V. Schwarzbold, N. Singh, A. Smith, R. Song, M. D. Snape, E. Sprinz, R. K. Sutherland, R. Tarrant, E. C. Thomson, M. E. Torok, M. Toshner, D. P. J. Turner, J. Vekemans, T. L. Villafana., M. E. E. Watson, C. J. Williams, A. D. Douglas, A. V. S. Hill, T. Lambe, S. C. Gilbert, A. J. Pollard and C. V. T. G. Oxford (2021). “Safety and efficacy of the ChAdOx1 nCoV-19 vaccine (AZD1222) against SARS-CoV-2: an interim analysis of four randomised controlled trials in Brazil, South Africa, and the UK.” Lancet 397(10269): 99-111.
Wherry, E. J., V. Teichgraber, T. C. Becker, D. Masopust, S. M. Kaech, R. Antia, U. H. von Andrian and R. Ahmed (2003). “Lineage relationship and protective immunity of memory CD8 T cell subsets.” Nat Immunol 4(3): 225-234.
Woods, J. A., M. A. Ceddia, M. D. Zack, T. W. Lowder and Q. Lu (2003). “Exercise training increases the naive to memory T cell ratio in old mice.” Brain Behav Immun 17(5): 384-392.
Wouters, O. J., K. C. Shadlen, M. Salcher-Konrad, A. J. Pollard, H. J. Larson, Y. Teerawattananon and M. Jit (2021). “Challenges in ensuring global access to COVID-19 vaccines: production, affordability, allocation, and deployment.” Lancet (London, England) 397(10278): 1023-1034.
Xia, X., Y. Zhang, C. R. Zieth and S. C. Zhang (2007). “Transgenes delivered by lentiviral vector are suppressed in human embryonic stem cells in a promoter-dependent manner.” Stem Cells Dev 16(1): 167-176.
Yokozeki, H., I. Katayama, O. Ohki, M. Arimura, K. Takayama, T. Matsunaga, T. Satoh, T. Umeda, M. Azuma, K. Okumura and K. Nishioka (1997). “Interferon-gamma differentially regulates CD80 (B7-1) and CD86 (B7-2/B70) expression on human Langerhans cells.” Br J Dermatol 136(6): 831-837.
Yu, J., L. H. Tostanoski, L. Peter, N. B. Mercado, K. McMahan, S. H. Mahrokhian, J. P. Nkolola, J. Liu, Z. Li, A. Chandrashekar, D. R. Martinez, C. Loos, C. Atyeo, S. Fischinger, J. S. Burke, M. D. Slein, Y. Chen, A. Zuiani, F. J. N. Lelis, M. Travers, S. Habibi, L. Pessaint, A. Van Ry, K. Blade, R. Brown, A. Cook, B. Finneyfrock, A. Dodson, E. Teow, J. Velasco, R. Zahn, F. Wegmann, E. A. Bondzie, G. Dagotto, M. S. Gebre, X. He, C. Jacob-Dolan, M. Kirilova, N. Kordana, Z. Lin, L. F. Maxfield, F. Nampanya, R. Nityanandam, J. D. Ventura, H. Wan, Y. Cai, B. Chen, A. G. Schmidt, D. R. Wesemann, R. S. Baric, G. Alter, H. Andersen, M. G. Lewis and D. H. Barouch (2020). “DNA vaccine protection against SARS-CoV-2 in rhesus macaques.” Science 369(6505): 806-811.
Zhao, J., J. Zhao, A. K. Mangalam, R. Channappanavar, C. Fett, D. K. Meyerholz, S. Agnihothram, R. S. Baric, C. S. David and S. Perlman (2016). “Airway Memory CD4(+) T Cells Mediate Protective Immunity against Emerging Respiratory Coronaviruses.” Immunity 44(6): 1379-1391.
Zhao, J., S. Zhao, J. Ou, J. Zhang, W. Lan, W. Guan, X. Wu, Y. Yan, W. Zhao, J. Wu, J. Chodosh and Q. Zhang (2020). “COVID-19: Coronavirus Vaccine Development Updates.” Frontiers in immunology 11: 602256-602256.
Zhou, R., K. K. To, Y. C. Wong, L. Liu, B. Zhou, X. Li, H. Huang, Y. Mo, T. Y. Luk, T. T. Lau, P. Yeung, W. M. Chan, A. K. Wu, K. C. Lung, O. T. Tsang, W. S. Leung, I. F. Hung, K. Y. Yuen and Z. Chen (2020). “Acute SARS-CoV-2 Infection Impairs Dendritic Cell and T Cell Responses.” Immunity 53(4): 864-877 e865.
Zhu, F. C., Y. H. Li, X. H. Guan, L. H. Hou, W. J. Wang, J. X. Li, S. P. Wu, B. S. Wang, Z. Wang, L. Wang, S. Y. Jia, H. D. Jiang, L. Wang, T. Jiang, Y. Hu, J. B. Gou, S. B. Xu, J. J. Xu, X. W. Wang, W. Wang and W. Chen (2020). “Safety, tolerability, and immunogenicity of a recombinant adenovirus type-5 vectored COVID-19 vaccine: a dose-escalation, open-label, non-randomised, first-in-human trial.” Lancet 395(10240): 1845-1854.
1. Korber, B., et al., 2020, Cell 182:812-827 e19
2. Isabel, S., et al., 2020, Sci Rep 10:14031
3. Plante, J. A., et al., 2021, Cell Host Microbe 29:508-515
4. World Health Organization (WHO). Tracking SARS-CoV-2 variants. [cited 2022 Jun. 6]; Available from: who.int/en/activities/tracking-SARS-CoV-2-variants/.
5. Ostrov, D. A. and G. W. Knox, 2022, Biochem Biophys Res Commun 586:87-92
6. Pouwels, K. B., et al., 2021, Nat Med 27:2127-2135
7. Lipsitch, M., et al., 2022, Nat Rev Immunol 22:57-65
8. Planas, D., et al., 2022, Nature 602:671-675
9. Planas, D., et al., 2021, Nature 596:276-280
10. Tegally, H., et al., 2022, Nat Med
11. Flemming, A., 2022, Nat Rev Immunol 22:75
12. Hirabara, S. M., et al., 2021, Front Cell Infect Microbiol 11:781429
13. Dejnirattisai, W., et al., 2022, Cell
14. Upadhyay, V., et al., 2021, J Biol Chem 297:101208
15. Lopez, M. V., et al., 2021, Vaccines (Basel) 9:1106
16. Wesemann, D. R., 2022, Cell
17. Salehi-Vaziri, M., et al., 2022, Arch Virol 167:327-344
18. Johnson, A. G., et al., 2022, MMWR. Morbidity and Mortality Weekly Report 71:132-138
19. Mizrahi, B., et al., 2021, Nat Commun 12:6379
20. Watson, C., 2022, Nature
21. Mercado, N. B., et al., 2020, Nature
22. Bos, R., et al., 2020, NPJ Vaccines 5:91
23. Amanat, F., et al., 2021, PLoS Biol 19:e3001384
24. López-Cort\tilde{e}s, G. I., et al., 2022, Vaccines 10:864
25. Liu, C., et al., 2021, Cell
26. Zhu, F. C., et al., 2020, Lancet 396:479-488
27. Zhu, F. C., et al., 2020, Lancet 395:1845-1854
28. Ramasamy, M. N., et al., 2020, Lancet
29. Binder, A. M., et al., 2017, MMWR Morb Mortal Wkly Rep 66:1039-1042
30. Sayedahmed, E. E., et al., 2018, Vaccine 36:6744-6751
31. Sester, M., et al., 2002, J Infect Dis 185:1379-87
32. Gangavarapu, K., et al., 2022, medRxiv 2022.01.27.22269965
33. Nie, J., et al., 2020, Nat Protoc 15:3699-3715
34. Zheng, J., et al., 2021, Nature 589:603-607
35. Oladunni, F. S., et al., 2020, Nat Commun 11:6122
36. McCray, P.B., Jr., et al., 2007, J Virol 81:813-21

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A chimeric, replication incompetent adenoviral vector comprising:
a) a hybrid promoter comprising an exogenous intron;
b) a nucleic acid sequence encoding a viral antigen operatively linked to the hybrid promoter;
c) a post-transcriptional regulatory element; and
d) a modified fiber protein comprising one or more domains from a first serotype adenovirus and a fiber knob domain from a second serotype adenovirus.

2. (canceled)

3. The adenoviral vector of claim 1, wherein the first serotype adenovirus is a serotype 5 adenovirus and wherein the second serotype adenovirus is a serotype 3 adenovirus.

4. The adenoviral vector of claim 1, wherein the hybrid promoter comprises a cytomegalovirus (CMV) immediate early enhancer, a β-actin promoter, and a chimeric intron.

5. The adenoviral vector of claim 4, wherein the β-actin promoter is a chicken β-actin promoter, and/or wherein the chimeric intron comprises a splice donor site from a β-actin gene and a splice acceptor site from a parvovirus.

6. (canceled)

7. (canceled)

8. (canceled)

9. (canceled)

10. The adenoviral vector of claim 1, wherein the post-transcriptional regulatory element comprises a woodchuck hepatitis virus post-transcriptional regulatory element (WPRE).

11. The adenoviral vector of claim 1, wherein the hybrid promoter comprises a nucleic acid sequence having at least 80% sequence identity with SEQ ID NO: 1, SEQ ID NO: 2, SEQ ID NO: 3, or SEQ ID NO: 4, or wherein the hybrid promoter comprises a nucleic acid sequence having at least 90% sequence identity with SEQ ID NO: 1, SEQ ID NO: 2, SEQ ID NO: 3, or SE ID NO: 4.

12. (canceled)

13. The adenoviral vector of claim 11, wherein the hybrid promoter comprises the nucleic acid sequence of SEQ ID NO: 1, SEQ ID NO: 2, SEQ ID NO: 3, or SEQ ID NO: 4.

14. The adenoviral vector of claim 1, further comprising a nucleic acid sequence encoding an immune modulator, wherein the nucleic acid sequence encoding the immune modulator is operatively linked an internal ribosome entry site (IRES) downstream of the viral antigen.

15. (canceled)

16. (canceled)

17. The adenoviral vector of claim 14, wherein the immune modulator is CD40 ligand (CD40L).

18. The adenoviral vector of claim 1, wherein the viral antigen is from a coronavirus, Zika virus, influenza virus, Ebola virus, Dengue virus, West Nile Virus, Lassa virus, Nipah virus, Rift Valley fever virus (RVFV), yellow fever, or Chikungunya virus.

19. (canceled)

20. (canceled)

21. (canceled)

22. The adenoviral vector of claim 18, wherein the viral antigen is a coronavirus spike (S) protein.

23. The adenoviral vector of claim 1, wherein the viral antigen is a coronavirus spike (S) protein comprising an amino acid sequence having at least 90% sequence identity to the amino acid sequence of SEQ ID NO: 14

24. The adenoviral vector of claim 23, wherein the spike protein comprises an amino acid sequence having at least one mutation relative to SEQ ID NO: 14, wherein the at least one mutation comprises;
a) D614G;
b) D614G, K986P, and V987P;
c) D614G and one of P681R and P68H1;
d) D614, K986P, V987P, and one of P681R and P681H;
e) D614G, and one or more of R682G, R683S and R685S;
f) D614G, K986P, V987P, and one or more of R682G, R683S, and R685S;
g) D614G, one of P681R and P68H1, and one or more of R682G, R683S and R685S;
h) D614G, K986P, V987P, one of P681R and P681H, and one or more of R682G, R683S and R685S; or
i) D614G, K986P, V987P, R681G, R683S, and R685S.

25. (canceled)

26. (canceled)

27. (canceled)

28. (canceled)

29. The adenoviral vector of claim 24, wherein the spike protein further comprises a K417T mutation or a K417N mutation.

30. (canceled)

31. (canceled)

32. The adenoviral vector of claim 24, wherein the nucleic acid sequence encoding the spike protein comprises a nucleic acid sequence having at least 80% sequence identity to SEQ ID NO: 5, SEQ ID NO: 6, SEQ ID NO: 7, SEQ ID NO: 8, SEQ ID NO: 9, or SEQ ID NO: 10, SEQ ID NO: 15, SEQ ID NO: 17, SEQ ID NO: 19, SEQ ID NO: 21, SEQ ID NO: 23, SEQ ID NO: 25, or SEQ ID NO: 27, or wherein the nucleic acid sequence encoding the spike protein comprises a nucleic acid sequence having at least 90% sequence identity to SEQ ID NO: 5, SEQ ID NO: 6, SEQ ID NO: 7, SEQ ID NO: 8, SEQ ID NO: 9, or SEQ ID NO: 10, SEQ ID NO: 15, SEQ ID NO: 17, SEQ ID NO: 19, SEQ ID NO: 21, SEQ ID NO: 23, SEQ ID NO: 25, or SEQ ID NO: 27.

33. (canceled)

34. The adenoviral vector of claim 32, wherein the nucleic acid sequence encoding the spike protein comprises the nucleic acid sequence of SEQ ID NO: 5, SEQ ID NO: 6, SEQ ID NO: 7, SEQ ID NO: 8, SEQ ID NO: 9, or SEQ ID NO: 10, SEQ ID NO: 15, SEQ ID NO: 17, SEQ ID NO: 19, SEQ ID NO: 21, SEQ ID NO: 23, SEQ ID NO: 25, or SEQ ID NO: 27.

35. (canceled)

36. A composition comprising the adenoviral vector of claim 1 and a pharmaceutically acceptable carrier, wherein the composition comprises about 1×10⁸ to about 1×10¹² viral particles (vp) of the adenoviral vector.

37. (canceled)

38. (canceled)

39. (canceled)

40. (canceled)

41. (canceled)

42. (canceled)

43. (canceled)

44. (canceled)

45. A method of inducing an immune response against a coronavirus in a subject, comprising administering to the subject the composition of claim 36.

46. The method of claim 45, wherein the subject is a human, a canine, or a feline.

47. (canceled)

48. The method of claim 45, wherein the method comprises administering to the subject a first dose of the composition of claim 36 as a prime, and administering to the subject a second dose of the composition of claim 36 as a boost, optionally wherein the first dose is administered to the subject by injection and the second dose is administered to the subject intranasally.

49. (canceled)

50. (canceled)

51. (canceled)

52. (canceled)
